SEQUENCE LISTING (157 sequences)

SEQ ID NO: 1 - DNA, 978 bp, Lactococcus lactis
  CDS (1)..(978): Lactococcus lactis subsp. cremoris MG1363 ldh gene encoding lactate dehydrogenase
atggctgataaacaacgtaagaaagttatccttgttggtgacggtgctgtaggttcatcatacgcttttgcccttgttaaccaaggaattgcacaagaattaggtattgttgacctttttaaagaaaaaactcaaggggatgcagaagacctttctcatgccttggcatttacatcacctaaaaagatttactctgcagactactctgatgcaagcgacgctgacctcgttgtcttgacttctggtgctccacaaaaaccaggtgaaactcgtcttgaccttgttgaaaaaaatcttcgtattactaaagatgttgtaactaaaattgttgcttcaggattcaaaggaatcttcctcgttgctgctaacccagttgacatcttgacatacgcaacttggaaattctctggtttccctaaaaaccgtgttgtaggttcaggtacttcacttgatactgcacgtttccgtcaagcattggctgaaaaagttgacgttgatgctcgttcaatccacgcatacatcatgggtgaacacggtgactcagaatttgctgtttggtcacacgctaacgttgctggtgttaaattggaacaatggttccaagaaaatgactaccttaacgaagcagaaatcgttgaattgtttgagtctgtacgtgatgcagcttactcaatcatcgctaaaaaaggtgcaacattctacggtgtggctgtagcccttgctcgtattactaaagcaattcttgatgatgaacatgcagtacttcctgtatcagtattccaagatggacaatatggggtaagcgactgctaccttggtcaaccagctgtagttggtgctgaaggtgttgttaacccaattcacattccattgaacgatgctgaaatgcaaaaaatggaagcttctggagctcaattgaaagctatcatcgatgaagcttttgctaaagaagaatttgcttctgcagttaaaaactaa

SEQ ID NO: 2 - protein, 325 aa, Lactococcus lactis
MADKQRKKVILVGDGAVGSSYAFALVNQGIAQELGIVDLFKEKTQGDAEDLSHALAFTSPKKIYSADYSDASDADLVVLTSGAPQKPGETRLDLVEKNLRITKDVVTKIVASGFKGIFLVAANPVDILTYATWKFSGFPKNRVVGSGTSLDTARFRQALAEKVDVDARSIHAYIMGEHGDSEFAVWSHANVAGVKLEQWFQENDYLNEAEIVELFESVRDAAYSIIAKKGATFYGVAVALARITKAILDDEHAVLPVSVFQDGQYGVSDCYLGQPAVVGAEGVVNPIHIPLNDAEMQKMEASGAQLKAIIDEAFAKEEFASAVKN

SEQ ID NO: 3 - DNA, 972 bp, Lactobacillus acidophilus
  CDS (1)..(972): Lactobacillus acidophilus-NCFM LBA0271 gene encoding lactate dehydrogenase
atggcaagagttgaaaaacctcgtaaagttattttagttggtgacggtgctgtaggttctacctttgcattttcaatggtgcaacaaggtattgctgaagaattaggtatcattgatattgctaaggaacacgttgaaggtgacgcaatcgacttagcagatgctactccatggactttcccaaagaacatttacgcagctgactacgctgactgcaaggacgcagacttagtagttattactgctggtgctccacaaaagccaggtgaaactcgtcttgaccttgttaacaagaacttgaagattttatcatcaatcgttgaaccagttgttgaatcaggctttgaaggtatcttcttagtagttgctaacccagttgacatcttgactcacgcaacttggaagatttcaggcttccctaaggatcgcgttattggttcaggtacttcacttgatactggtcgtcttcaaaaggttatcggtaagatggaacacgttgacccacgttcagttaatgcatacatgcttggtgaacacggtgatactgaattcccagtatggagctacaacaatgttggtggcgtaaaggttagcgactgggttaaggctcacggtatggatgaatctaagcttgaagaaatccacaaggaagttgctgacatggcttacgacattatcaacaagaagggtgctactttctacggtatcggtacagcttcagcaatgatcgctaaggctatcttgaacgatgaacaccgtgtacttccactctcagttgcaatggatggtcaatacggtttacacgaccttcacattggtactcctgcagttgttggccgtaacggtcttgaacaaattattgaaatgcctttaaccgctgatgaacaagctaagatggaagcttctgctaagcaattaaaggaagttatggacaaagcctttgaagaaactggcgttaaggttcgtcaataa

SEQ ID NO: 4 - protein, 323 aa, Lactobacillus acidophilus
MARVEKPRKVILVGDGAVGSTFAFSMVQQGIAEELGIIDIAKEHVEGDAIDLADATPWTFPKNIYAADYADCKDADLVVITAGAPQKPGETRLDLVNKNLKILSSIVEPVVESGFEGIFLVVANPVDILTHATWKISGFPKDRVIGSGTSLDTGRLQKVIGKMEHVDPRSVNAYMLGEHGDTEFPVWSYNNVGGVKVSDWVKAHGMDESKLEEIHKEVADMAYDIINKKGATFYGIGTASAMIAKAILNDEHRVLPLSVAMDGQYGLHDLHIGTPAVVGRNGLEQIIEMPLTADEQAKMEASAKQLKEVMDKAFEETGVKVRQ

SEQ ID NO: 5 - DNA, 915 bp, Lactobacillus acidophilus
  CDS (1)..(915): Lactobacillus acidophilus-NCFM LBA1516 gene encoding lactate dehydrogenase
atgagaaaagtaggtataatcggaatgggtcatgtaggtgctactgtagcatatactttgtttactcatggaattgctgacgaattagttttaattgataaaaatgaagataaagttgctgctgaatataatgacttacgtgattctttatcaagaaataattactacgttcgtgtaactatgcaagattggcatgaattaaaagatgcggatattattgtaactgcttttggtgatattgcggctagtgtcaagactggcgatcgttttggtgaatttgaacttaacgctaagaatgcaaaagaagtcggtgaaaagattaagaacactggttttaagggtgtacttcttaatatctctaatccatgtgatgctgttgcacaaatcttgcaagaaactactggattaagtaagaaccaagttttaggtactggtactttcttggatactgcaagaatgcaaagaattattggtgaaaagctaggtcaagatccaaaaaatgttgaaggttgggttttgggtgaacatggttcatcacaatttatcgcttggtccactgttcgtgttaacaacaagattgctattcaattatttagcgaaaatgaacaaagaaagttaaataaggtaccaaacaagaattcatttgtggttgcaaatggaaaaggctacactagttacgcaattgctacatgtgctgtaaggcttattcaagcaatcttttctgatgctcatttatatgctcctgtatcagtatacaatccagaatacaagacttatattggctatcctgcaattattggtcgtgatggaattgaacaagtgattgaattgaaacttacttcaaatgaacgtgaaaagcttcaagcagcagctgataagatcaaggaacacttggaacaactgaagaacaagtaa

SEQ ID NO: 6 - protein, 304 aa, Lactobacillus acidophilus
MRKVGIIGMGHVGATVAYTLFTHGIADELVLIDKNEDKVAAEYNDLRDSLSRNNYYVRVTMQDWHELKDADIIVTAFGDIAASVKTGDRFGEFELNAKNAKEVGEKIKNTGFKGVLLNISNPCDAVAQILQETTGLSKNQVLGTGTFLDTARMQRIIGEKLGQDPKNVEGWVLGEHGSSQFIAWSTVRVNNKIAIQLFSENEQRKLNKVPNKNSFVVANGKGYTSYAIATCAVRLIQAIFSDAHLYAPVSVYNPEYKTYIGYPAIIGRDGIEQVIELKLTSNEREKLQAAADKIKEHLEQLKNK

SEQ ID NO: 7 - DNA, 927 bp, Lactobacillus acidophilus
  CDS (1)..(927): Lactobacillus acidophilus-NCFM LBA0910 gene encoding lactate dehydrogenase
atgagtagaaaagtgtttcttgtaggtgatggtgctgttggttcaacttttgcaaatgacttattgcaaaatacaactgttgatgaattagcgatttttgatgttgctaaagatcgtccagttggtgattcaatggatttggaagatattactccatttacaggtcaaactaatattcatccagcagaatatagtgatgctaaagatgcagatgtgtgtgtaattactgctggtgttcctcgtaaacctggtgaaactagacttgacttagttaataagaatgtaaagattttaaagactattgttgatccggttgttgaatccggttttaagggtgtatttgttgtttcagctaacccggttgatattttaaccacattgactcaaaaaatatccggttttccaaaagatcgtgtaattggtactggtacttcacttgattcaatgcgtcttcgcgttgaattggcaaagaaacttaatgttccagtagctaaggttaactcaatggttcttggtgaacacggtgatactagttttgaaaactttgacgaatcaactgttgacaataagccacttcgcgattactcagaaatcaatgataatgttttaagtgaaattgagtcagacgtccgtaaaaagggtggaaagatcatcactaacaaaggagctacattctatggtgttgctatgatgcttactcaaattgttagtgctattttagataatcgttcaatttgtttgccattatcagccccaattaatggtgaatatggcattaagcatgatctttacttaggtactccaactataattaacggtaatggtattgaaaaagttattgaaactaaactttcagatgtagaaaaagctaagatgatcaattctgcagataagatgcaagaagttttatcaggtgttgaaatgtaa

SEQ ID NO: 8 - protein, 308 aa, Lactobacillus acidophilus
MSRKVFLVGDGAVGSTFANDLLQNTTVDELAIFDVAKDRPVGDSMDLEDITPFTGQTNIHPAEYSDAKDADVCVITAGVPRKPGETRLDLVNKNVKILKTIVDPVVESGFKGVFVVSANPVDILTTLTQKISGFPKDRVIGTGTSLDSMRLRVELAKKLNVPVAKVNSMVLGEHGDTSFENFDESTVDNKPLRDYSEINDNVLSEIESDVRKKGGKIITNKGATFYGVAMMLTQIVSAILDNRSICLPLSAPINGEYGIKHDLYLGTPTIINGNGIEKVIETKLSDVEKAKMINSADKMQEVLSGVEM

SEQ ID NO: 9 - DNA, 924 bp, Lactobacillus delbrueckii
  CDS (1)..(924): Lactobacillus delbrueckii subsp. bulgaricus ATCC 11842_ Ldb0120 gene encoding lactate dehydrogenase
atgagtagaaaagtcctgctggttggcgacggtgctgtcggttccaactttgccaacgatttattgcaaacaacgcgggtggatgaactggtcatctgcgacttgaataaagaccgggccgcgggcgactgcctggacctggaagacatgacctactttacgggccagaccaagctgagagctggtgactacagcgacgcggctgacgccgacgtggtagttatcacggccggcgtgccccgcaagccaggtgaaagccggcttgacctgatcaagaagaatgaagccatcttgcggagcatcgttgatccagtcgtagcttccggcttcagcggcatcttcgtcgtttccgccaacccggttgacatcttgaccactttgacccagaagctgtccggcttcccaaagaagcgggtcatcgggaccgggacttccctggactcagctagtttgcgggtagaactggccaagcggctgcaagtgccaattgaatcagtcaacgcctgggttctgggtgaacatggcgacagcagctttgagaacttctcaagtgccgtggtcaacggcaagcccttgcttgactatccgggcatgacagaagcagccttagatgagattgaagcccacgtccgggaaaagggcagcgagattatcgtcaaaaagggcgcaacctactatggcgtggccatgatgctggccaagatcgtgacggccattttggaaaacaacgacctggccctgcctctttccgcaccattgcacggggaatacgggatcaaggatgagatctacctgggcaccctggccatcatcaacggacaagggatcagccacgtactggaattgccgttaaatgacagcgaattggccaagatgcgggcctcagccgcgacgataaaggcaaccctggactctttaggataa

SEQ ID NO: 10 - protein, 307 aa, Lactobacillus delbrueckii
MSRKVLLVGDGAVGSNFANDLLQTTRVDELVICDLNKDRAAGDCLDLEDMTYFTGQTKLRAGDYSDAADADVVVITAGVPRKPGESRLDLIKKNEAILRSIVDPVVASGFSGIFVVSANPVDILTTLTQKLSGFPKKRVIGTGTSLDSASLRVELAKRLQVPIESVNAWVLGEHGDSSFENFSSAVVNGKPLLDYPGMTEAALDEIEAHVREKGSEIIVKKGATYYGVAMMLAKIVTAILENNDLALPLSAPLHGEYGIKDEIYLGTLAIINGQGISHVLELPLNDSELAKMRASAATIKATLDSLG

SEQ ID NO: 11 - DNA, 939 bp, Lactobacillus casei
  CDS (1)..(939): Lactobacillus casei ATCC 334, LSEI_0634 gene encoding lactate dehydrogenase
atgcggaacaacggcaatattattttaattggtgatggcgcgattggatcaagttatgcgttcaactgtctcaccaccggcgtcggccagagtcttggtattattgatgttaacgaaaaacgcgtgcaaggtgacgtcgaagatctttctgatgccctaccttacacgtcacagaagaatatttatgcggccagctatgaagattgcaaatatgccgatattatcgtcatcacagccggaattgcccaaaagccaggtcagacacgcctacaacttttggccatcaacgcaaagatcatgaaagaaatcacacataacattatggcaagcgggttcaatgggtttattttagtcgcatccaatccagtcgatgtccttgccgaattagtcttgcaagaatccggcttgccacgtaatcaggtgctgggatcagggacggcgcttgattcagctcgcttacggtctgagatcggtctgcgttataacgtggacgcccggattgtgcacggctacatcatgggtgaacacggcgattctgaatttccagtttgggactacaccaacattggcggcaaaccgattcttgattggattcctaaagatcgccaggataaagatctgcctgatattagcgagcgcgtcaaaacggctgcttatggcatcattgagaaaaaaggtgccactttctacggcattgctgcctcattaacccgtttaaccagcgccttcttgaatgatgatcgcgcagcattcgcaatgtccgtccatttggaaggcgaatacggcttgtcaggtgtttccatcggtgtaccggtaatcctcggcgctaatggcttagaacgcatcattgagctggatttgaacccagaagatcataagcggttagccgattctgcagctattttgaaagaaaatctgaagaaggctcaagaagcttaa

SEQ ID NO: 12 - protein, 312 aa, Lactobacillus casei
MRNNGNIILIGDGAIGSSYAFNCLTTGVGQSLGIIDVNEKRVQGDVEDLSDALPYTSQKNIYAASYEDCKYADIIVITAGIAQKPGQTRLQLLAINAKIMKEITHNIMASGFNGFILVASNPVDVLAELVLQESGLPRNQVLGSGTALDSARLRSEIGLRYNVDARIVHGYIMGEHGDSEFPVWDYTNIGGKPILDWIPKDRQDKDLPDISERVKTAAYGIIEKKGATFYGIAASLTRLTSAFLNDDRAAFAMSVHLEGEYGLSGVSIGVPVILGANGLERIIELDLNPEDHKRLADSAAILKENLKKAQEA

SEQ ID NO: 13 - DNA, 981 bp, Lactobacillus casei
  CDS (1)..(981): Lactobacillus casei ATCC 334 _ gene LSEI_2549 encoding a lacate dehydrogenase
  tRNA (1)..(3): Met is inserted by tRNA at start codon (GTG) instead of Val
gtggcaagtattacggataaggatcaccaaaaagttattctcgttggtgacggcgccgttggttcaagttatgcctacgcaatggttttgcaaggtatcgctcaggaaatcggaatcgttgacattttcaaggacaagacaaagggtgacgcgattgacttgagcaacgcgctcccattcacaagtcctaagaagatttattcagctgaatacagcgatgctaaggatgctgatctggttgttatcacagctggcgctcctcagaagcctggcgaaactcgtttggacttggttaacaagaacttgaagatcttgaagtccattgttgacccaatcgttgattccggctttaacggtattttcttagttgctgccaacccagttgacattttgacctatgcaacttggaaactttctggcttcccgaagaaccgggttgttggttccggtacttcactggacaccgcccgcttccgtcagtccattgctgaaatggttaatgttgacgctcgttcggtccacgcttacatcatgggcgaacatggtgacactgaattccctgtatggtcccacgctaacattggtggcgttaccatcgctgaatgggttaaggctcatccagaaatcaaggaagacaagcttgttaagatgtttgaagacgttcgtgacgccgcttatgaaatcatcaaactcaagggtgcgaccttctatggtatcgcaactgcccttgcccggatttcaaaggcaatccttaacgacgaaaatgcggttctgccactttccgtttacatggatggtcaatatggcttgaacgacatctacatcggtactccagctgtgatcaaccgtaatggtatccagaacatcctggaaatcccattgaccgatcacgaagaagaatccatgcagaaatctgcttctcaattgaagaaggttctgaccgatgctttcgctaagaatgacatcgaaactcgtcagtaa

SEQ ID NO: 14 - protein, 326 aa, Lactobacillus casei
VASITDKDHQKVILVGDGAVGSSYAYAMVLQGIAQEIGIVDIFKDKTKGDAIDLSNALPFTSPKKIYSAEYSDAKDADLVVITAGAPQKPGETRLDLVNKNLKILKSIVDPIVDSGFNGIFLVAANPVDILTYATWKLSGFPKNRVVGSGTSLDTARFRQSIAEMVNVDARSVHAYIMGEHGDTEFPVWSHANIGGVTIAEWVKAHPEIKEDKLVKMFEDVRDAAYEIIKLKGATFYGIATALARISKAILNDENAVLPLSVYMDGQYGLNDIYIGTPAVINRNGIQNILEIPLTDHEEESMQKSASQLKKVLTDAFAKNDIETRQ

SEQ ID NO: 15 - DNA, 906 bp, Lactobacillus casei
  CDS (1)..(906): Lactobacillus casei ATCC 334_gene LSEI_2607 encoding lactate dehydrogenase
atggcaagaacaattggtattattggtattggacacgtaggtgtgaccacggcttttaatcttgtcagcaaaggggttgctgacaagttagtcttgattgataaaaaggctgaacttgctgaaggcgaaagctttgacttgaaggatgcgcttggcggcttgcccacttatactgacattgttgtcaacgattatgatgctttgaaagatgctgatgtggttatttccgcggtcggcaatattggcgccatttcaaatggtgatcgaattggagaaaccaagacttcaaaagtagcactagatgacgtcgctccaaaactgaaggcgtctggcttccatggcgttttgctagatatcaccaatccatgtgatgctgtcacgagttactggcaatatctcctagatttaccgaagtctcaaattattgggactgggacttcgctggatacttatcggatgcgacgggcggttgccgataccttgcatgtgaatgttgctgacgtgcgtggttacaatatgggcgaacatggcgaatcgcaatttaccgcttggtcaacagtacgggtcaataatgaaccgattgctgaatatgcgaaggttgattacgaccagcttgccgatgatgcgcgggcaggcggttggaagatttatcaggctaagcattatacgagctacggtatcgccaccatcgcgactgaaatgacacaggcgatcatcagcgatgcacatcgcatcttcccatgcgctaattacgatccagaattcggtattgctatcggtcatccagccatgatcggtaaacaaggtgttatcaagacgccaacgttgaagttgacagatgaagagcgtgctaagtatgttcactctgccggtatcattaaggatacctttgaaaaaatgaagtag

SEQ ID NO: 16 - protein, 301 aa, Lactobacillus casei
MARTIGIIGIGHVGVTTAFNLVSKGVADKLVLIDKKAELAEGESFDLKDALGGLPTYTDIVVNDYDALKDADVVISAVGNIGAISNGDRIGETKTSKVALDDVAPKLKASGFHGVLLDITNPCDAVTSYWQYLLDLPKSQIIGTGTSLDTYRMRRAVADTLHVNVADVRGYNMGEHGESQFTAWSTVRVNNEPIAEYAKVDYDQLADDARAGGWKIYQAKHYTSYGIATIATEMTQAIISDAHRIFPCANYDPEFGIAIGHPAMIGKQGVIKTPTLKLTDEERAKYVHSAGIIKDTFEKMK

SEQ ID NO: 17 - DNA, 963 bp, Lactobacillus plantarum
  CDS (1)..(963): Lactobacillus plantarum WCFS1_ gene lp_0537 encoding lactate dehydrogenase
  tRNA (1)..(3): Met is inserted by tRNA at start codon (TTG) instead of Leu
ttgtcaagcatgccaaatcatcaaaaagttgtgttagtcggcgacggcgctgttggttctagttacgcttttgccatggcacaacaaggaattgctgaagaatttgtaattgtcgatgttgttaaagatcggacaaagggtgacgcccttgatcttgaagacgcccaagcattcaccgctcccaagaagatttactcaggcgaatattcagattgtaaggacgctgacttagttgttattacagccggtgcgcctcaaaagcctggtgaatcacgtttagacttagttaacaagaatttaaatatcctatcatccattgtcaaaccagttgttgactccggctttgacggcatcttcttagttgctgctaaccctgttgacatcttaacttacgctacttggaaattctcaggtttcccaaaggatcgtgtcattggttcagggacttccttagactcttcacgtttacgcgttgcgttaggcaaacaattcaatgttgatcctcgttccgttgatgcttacatcatgggtgaacacggtgattctgaatttgctgcttactcaactgcaaccatcgggacacgtccagttcgcgatgtcgctaaggaacaaggcgtttctgacgaagatttagccaagttagaagacggtgttcgtaacaaagcttacgacatcatcaacttgaagggtgccacgttctacggtatcgggactgctttaatgcggatttccaaagccattttacgtgatgaaaatgccgttttaccagtaggtgcctacatggacggccaatacggcttaaacgacatttatatcgggactccggctgtgattggtggaactggtttgaaacaaatcatcgaatcaccactttcagctgacgaactcaagaagatgcaagattccgccgcaactttgaaaaaagtgcttaacgacggtttagctgaattagaaaataaataa

SEQ ID NO: 18 - protein, 320 aa, Lactobacillus plantarum
LSSMPNHQKVVLVGDGAVGSSYAFAMAQQGIAEEFVIVDVVKDRTKGDALDLEDAQAFTAPKKIYSGEYSDCKDADLVVITAGAPQKPGESRLDLVNKNLNILSSIVKPVVDSGFDGIFLVAANPVDILTYATWKFSGFPKDRVIGSGTSLDSSRLRVALGKQFNVDPRSVDAYIMGEHGDSEFAAYSTATIGTRPVRDVAKEQGVSDEDLAKLEDGVRNKAYDIINLKGATFYGIGTALMRISKAILRDENAVLPVGAYMDGQYGLNDIYIGTPAVIGGTGLKQIIESPLSADELKKMQDSAATLKKVLNDGLAELENK

SEQ ID NO: 19 - DNA, 930 bp, Lactobacillus plantarum
  CDS (1)..(930): Lactobacillus plantarum WCFS1; gene lp_1101 encoding lactate dehydrogenase
atggataagaagcaacgcaaagtcgtaattgttggtgatggctcggtgggttcatcatttgccttttcattggtccaaaattgcgccctagatgaactcgttatcgttgacttggttaaaacgcacgcagagggggacgttaaggatttggaagatgttgccgcctttacgaatgcgaccaacattcataccggtgaatatgcggatgcgcgtgatgctgacatcgttgtcattacggctggtgtgcctcgtaagcctggtgagagtcgtttagatttgattaaccgcaatacgaagattctggaatccatcgtcaaaccagtggttgcgagtggttttaatggttgcttcgttatctcaagtaatcccgtcgatattttgacttcgatgacgcaacgtttatccggttttccacggcatcgggtcattggtaccgggacttccttggatacggcgcggttacgggtcgccttggctcagaagttgaatgttgccaccactgcagttgatgctgcggtacttggagaacatggtgatagttccatcgttaattttgatgaaattatgatcaatgctcagcccttaaagacggtcacaacggtcgatgatcagttcaaagctgaaatcgagcaagctgttcgtggtaaaggtggtcaaatcattagtcagaagggggccacgttctatggggtcgccgttagtttgatgcaaatctgccgagcaattttgaacgatgaaaatgctgagttgattgtctccgccgctttgtctggtcaatatggcattaacgatttgtacttggggtcacccgccattattaaccgcaacgggctccaaaaagtgatcgaagctgagctatcagatgatgagcgtgcccggatgcaacatttcgcagccaagatgctgaccatgatgaatgtggcatcataa

SEQ ID NO: 20 - protein, 309 aa, Lactobacillus plantarum
MDKKQRKVVIVGDGSVGSSFAFSLVQNCALDELVIVDLVKTHAEGDVKDLEDVAAFTNATNIHTGEYADARDADIVVITAGVPRKPGESRLDLINRNTKILESIVKPVVASGFNGCFVISSNPVDILTSMTQRLSGFPRHRVIGTGTSLDTARLRVALAQKLNVATTAVDAAVLGEHGDSSIVNFDEIMINAQPLKTVTTVDDQFKAEIEQAVRGKGGQIISQKGATFYGVAVSLMQICRAILNDENAELIVSAALSGQYGINDLYLGSPAIINRNGLQKVIEAELSDDERARMQHFAAKMLTMMNVAS

SEQ ID NO: 21 - DNA, 963 bp, Pediococcus pentosaceus
  CDS (1)..(963): Pediococcus pentosaceus ATCC 25745 gene PEPE_1554 encoding lactate dehydrogenase
  tRNA (1)..(3): Met is inserted by tRNA at start codon (TTG) instead of Leu
ttgtctaaaattcaaaaccatcaaaaagttgtattagtcggcgacggcgctgtaggttcaagttatgcctttgcaatggcacaacaaggaattgcagaagaattcgtaatcgttgatgttgttaaggaccgtactgttggtgacgcacttgatcttgaagatgctactccatttactgcacctaagaacatttactcaggtgaatattctgactgtaaagatgcagatcttgtagttatcactgctggtgctccacaaaaaccaggtgaaacacgtcttgaccttgttaacaagaacttaaacattctttcaacaatcgttaagccagttgttgactcaggcttcgacggtatcttcttagttgctgctaacccagttgatatccttacttacgcaacatggaaattctcaggattccctaaagaaaaggttatcggttcaggtatttctcttgattcagcacgtctacgtgttgctcttggtaagaaattcaacgttagccctgactcagttgatgcatacatcatgggtgaacatggtgacagtgaattcgctgcttactcaactgcttcaatcggtacaaagccattgcttgatatcgctaaagaagaaggcgtttcaactgacgaattagctgaaatcgaagacagtgttcgtaacaaagcttacgaaatcatcaacaagaagggtgctacattctacggtgttggtactgctttgatgcgtatttcaaaagctatcttgcgtgacgaaaatgctgtattgcctgttggtgcttacatggacggcgaatatggtttgaatgacatctacatcggtacacctgctgttattaacggccaaggtttgaaccgtgttatcgaatcaccacttagtgatgacgaaatgaagaagatgacagattcagctacaacattgaaaaaagttcttacagatggtttgaaagctcttgaagaaaaataa

SEQ ID NO: 22 - protein, 320 aa, Pediococcus pentosaceus
LSKIQNHQKVVLVGDGAVGSSYAFAMAQQGIAEEFVIVDVVKDRTVGDALDLEDATPFTAPKNIYSGEYSDCKDADLVVITAGAPQKPGETRLDLVNKNLNILSTIVKPVVDSGFDGIFLVAANPVDILTYATWKFSGFPKEKVIGSGISLDSARLRVALGKKFNVSPDSVDAYIMGEHGDSEFAAYSTASIGTKPLLDIAKEEGVSTDELAEIEDSVRNKAYEIINKKGATFYGVGTALMRISKAILRDENAVLPVGAYMDGEYGLNDIYIGTPAVINGQGLNRVIESPLSDDEMKKMTDSATTLKKVLTDGLKALEEK

SEQ ID NO: 23 - DNA, 996 bp, Leuconostoc mesenteroides
  CDS (1)..(996): Leuconostoc mesenteroides subsp. mesenteroides ATCC 8293 gene LEUM_1756 encoding lactate dehydrogenase
atgaagatttttgcttacggcattcgtgatgatgaaaagccatcacttgaagaatggaaagcggctaacccagagattgaagtggactacacacaagaattattgacacctgaaacagctaagttggctgagggatcagattcagctgttgtttatcaacaattggactatacacgtgaaacattgacagctttagctaacgttggtgttactaacttgtcattgcgtaacgttggtacagataacattgattttgatgcagcacgtgaatttaactttaacatttcaaatgttcctgtttattcaccaaatgctattgcagaacactcaatgattcaattatctcgtttgctacgtcgcacgaaagcattggatgccaaaattgctaagcacgacttgcgttgggcaccaacaattggacgtgaaatgcgtatgcaaacagttggtgttattggtacaggtcatattggccgtgttgctattaacattttgaaaggctttggggccaaggttattgcttatgacaagtacccaaatgctgaattacaagcagaaggtttgtacgttgacacattagacgaattatatgcacaagctgatgcaatttcattgtatgttcctggtgtacctgaaaaccatcatctaatcaatgcagatgctattgctaagatgaaggatggtgtggttatcatgaacgctgcgcgtggtaatttgatggacattgacgctattattgatggtttgaattctggtaagatttcagacttcggtatggacgtttatgaaaatgaagttggcttgttcaatgaagattggtctggtaaagaattcccagatgctaagattgctgacttgattgcacgcgaaaatgtattggttacgccacacacggctttctatacaactaaagctgttctagaaatggttcaccaatcatttgatgcagcagttgctttcgccaagggtgagaagccagctattgctgttgaatattaa

SEQ ID NO: 24 - protein, 331 aa, Leuconostoc mesenteroides
MKIFAYGIRDDEKPSLEEWKAANPEIEVDYTQELLTPETAKLAEGSDSAVVYQQLDYTRETLTALANVGVTNLSLRNVGTDNIDFDAAREFNFNISNVPVYSPNAIAEHSMIQLSRLLRRTKALDAKIAKHDLRWAPTIGREMRMQTVGVIGTGHIGRVAINILKGFGAKVIAYDKYPNAELQAEGLYVDTLDELYAQADAISLYVPGVPENHHLINADAIAKMKDGVVIMNAARGNLMDIDAIIDGLNSGKISDFGMDVYENEVGLFNEDWSGKEFPDAKIADLIARENVLVTPHTAFYTTKAVLEMVHQSFDAAVAFAKGEKPAIAVEY

SEQ ID NO: 25 - DNA, 990 bp, Leuconostoc mesenteroides
  CDS (1)..(990): Leuconostoc mesenteroides subsp. mesenteroides ATCC 8293 gene LEUM_2043 encoding lactate dehydrogenase
atgaaaattttaatgtacaatacagtttcagacgaaatgccatatgttgaaaaatggactgaaaaaacaggccacgaagtagtgaccattgatcaaccacttaacgaaacgacggtggatcgcgctaaaggttttgatgcaattaccacacaacaaacaacagccattaatgcaccagtttatgcgcaactagctcaattcaatattaagcaaatcgctattcgacaggttggctatgatgtgttggacttgccagaagcctttgctcacaatattcgtttgtctaatgttgcagcctattcaccccgagcaattgcagaatacacgctaacacagctttttaatttaattcgacacaataaaaagtttgatcgcgccatgaatacgggtgactatacttgggctgctggtggtcaagcgagagaaattcatgatttaactgttgctgtgattggtgtaggtcgaattgggacagcccttgcagaaatgcttcacgcgctgggcgcaacagtgttaggtgtggacccaatatatcgtgcccagaacgaaccatttgtaacttatacgacattagatgatgctttgtctcgagcggatgttattagttttcatacaccattaaatgacgagacgcgctatatggctgatgaaaatttctttgctaaaattcagccaggaacaattatgttgaattttgcgcgtggtgaaattgttgacatggttgagcttgaaaaagcactcgaatcaaagttattggctggtgcagcatttgatgtgacaccaaatgaaaatgattttatgaaccaaaagcaagatataacggcgctaccagcagatgtaaagcgcttaatcgaatatgacaactttctactgtcaccacatattgctttttatacaaacacagcaataaaaaatatggtggaaatggctttaaatgatgccgtaacgatggtaaatggcggcgttactgaaaacgaaattttgcggtaa

SEQ ID NO: 26 - protein, 329 aa, Leuconostoc mesenteroides
MKILMYNTVSDEMPYVEKWTEKTGHEVVTIDQPLNETTVDRAKGFDAITTQQTTAINAPVYAQLAQFNIKQIAIRQVGYDVLDLPEAFAHNIRLSNVAAYSPRAIAEYTLTQLFNLIRHNKKFDRAMNTGDYTWAAGGQAREIHDLTVAVIGVGRIGTALAEMLHALGATVLGVDPIYRAQNEPFVTYTTLDDALSRADVISFHTPLNDETRYMADENFFAKIQPGTIMLNFARGEIVDMVELEKALESKLLAGAAFDVTPNENDFMNQKQDITALPADVKRLIEYDNFLLSPHIAFYTNTAIKNMVEMALNDAVTMVNGGVTENEILR

SEQ ID NO: 27 - DNA, 987 bp, Streptococcus thermophilus
  CDS (1)..(987): Streptococcus thermophilus CNRZ1066 gene str1280 encoding lactate dehydrogenase
atgactgcaactaaactacacaaaaaagtcatccttgttggtgacggtgccgtaggttcatcttacgctttcgcacttgtaaaccaaggtatcgctcaagaactaggtatcatcgaaattccacaattatttgaaaaagccgttggtgatgcgcttgaccttagccacgcacttcctttcacttcacctaaaaaaatctatgcagctaaatatgaagactgtgcggatgctgaccttgtagttatcactgctggtgctcctcaaaaaccaggtgagactcgtcttgatcttgttggtaaaaaccttgcaatcaacaaatcaatcgttactcaagttgttgaatcaggattcaacggtattttccttgtagctgctaacccagtagacgtattgacttactctacatggaagttctcaggattccctaaagaacgcgttatcggttcaggtacttcacttgactcagctcgtttccgtcaagcacttgctgaaaaacttaatgtcgatgctcgttcagttcacgcttacatcatgggtgaacacggcgactcagagtttgcggtttggtcacacgctaacatcgccggtgtaaaccttgaagagttccttaaagacaaagaaaacgttcaagaagctgaattagttgaattgttcgaaggtgttcgtgatgcagcttacacaattatcaacaaaaaaggtgctacatactacggtatcgcagtagcccttgctcgtatcactaaagctatccttgacgatgaaaatgcagtacttccattgtctgtattccaagaaggtcaatatggtgtaaacaacatctttatcggtcaacctgccattgtaggtgcacacggtatcgtacgtccagtaaacatcccattgaacgatgctgaacaacaaaagatgaaggcttctgccgatgaattgcaagctatcattgatgaagcatggaaaaaccctgaattccaagaagcttcaaaaaactaa

SEQ ID NO: 28 - protein, 328 aa, Streptococcus thermophilus
MTATKLHKKVILVGDGAVGSSYAFALVNQGIAQELGIIEIPQLFEKAVGDALDLSHALPFTSPKKIYAAKYEDCADADLVVITAGAPQKPGETRLDLVGKNLAINKSIVTQVVESGFNGIFLVAANPVDVLTYSTWKFSGFPKERVIGSGTSLDSARFRQALAEKLNVDARSVHAYIMGEHGDSEFAVWSHANIAGVNLEEFLKDKENVQEAELVELFEGVRDAAYTIINKKGATYYGIAVALARITKAILDDENAVLPLSVFQEGQYGVNNIFIGQPAIVGAHGIVRPVNIPLNDAEQQKMKASADELQAIIDEAWKNPEFQEASKN

SEQ ID NO: 29 - DNA, 960 bp, Oenococcus oeni
  CDS (1)..(960): Oenococcus oeni PSU-1 gene OEOE_0025 encoding lactate dehydrogenase
atgaaaaatgttctcatcacagcggagcttcctcaagttgcaaatagtatacttcaacaagccggcctaaatgtcgaaatttttacgggtgataaattaattacaaagaaggaattacttgataaagtcaaagataaagattttttaattacctctctatcaacagatgttgatgaagacgtaataaatgcagctccaaaattaaaattaattgcgaactttggagccggattcaataacatcgacataaatagtgctcgagcaaaagaaatatctgttacaaacactccttttgtctcgactacttcagtagccgaggtcacagcaggactaattatttccctatcgcatcgtttggttgaaggagataatttaatgcacgatcaaggattcaatggctggagtcccttattctttttaggccatgaactatctaaaaaaacattagggatcatcggcatgggtcagattggcaaggctttggccaaacgtatgcaggactttgatatgaaaattgtttacacacaaaggcatcacttaaaagaagatgaggaaaaagaacttcatgctgttttcttggaaaaagacgaattaattaaacaaagcgatgtaattagcttgcatttgccactaacgaaaaatacccatcacttgttgggtgctaaagaatttgcaactatgaaaaaaacttctttcctaattaacgctgctcgtggaccgttaattgacgaaaatgcattgctacagtccctaaaagaaaagcagcttgctggagctgctttagatgtttatgaacatgaaccaaaagttgatgatcaatttaaacagatgaaaaatgttattctgactccccatatcggcaatgcaaccatcgaagcccgtaatgcaatggctgaagtcgtcgccaaaaatgtggtatcagtgttaaatggtgaaaaaccgaaatatatcgttaattaa

SEQ ID NO: 30 - protein, 319 aa, Oenococcus oeni
MKNVLITAELPQVANSILQQAGLNVEIFTGDKLITKKELLDKVKDKDFLITSLSTDVDEDVINAAPKLKLIANFGAGFNNIDINSARAKEISVTNTPFVSTTSVAEVTAGLIISLSHRLVEGDNLMHDQGFNGWSPLFFLGHELSKKTLGIIGMGQIGKALAKRMQDFDMKIVYTQRHHLKEDEEKELHAVFLEKDELIKQSDVISLHLPLTKNTHHLLGAKEFATMKKTSFLINAARGPLIDENALLQSLKEKQLAGAALDVYEHEPKVDDQFKQMKNVILTPHIGNATIEARNAMAEVVAKNVVSVLNGEKPKYIVN

SEQ ID NO: 31 - DNA, 978 bp, Oenococcus oeni
  CDS (1)..(975): Oenococcus oeni PSU-1 gene OEOE_1709 encoding lactate dehydrogenase
atgtatgctgttttcgaagatgaagtaccttatattgaagagtttgccaatcgtactggccatgaaatcgttcaggaaaccgattatttatccgaaaaaaatgtcgatttggcagccggtttcgatgcggttgttacccaacagacggtcaacattcctgacatggtgtattctcgtctggctgaattaggtatcaggcaaatcagtatccgtcaggttggttacgatattttaaatatcgatgcgattgataaagccggactgcgtgcgtcaaatgtcgcagcttattcgccaagagcgattgccgaatacacattgacacaacttttaaacctgatccgtgacaacaagagatattatcaggccacagaatcgggtaattggttatggtctgcagcccctcaggggaaagaaatccatgatctaacggtcgctgttatcggcgctggacgtattggttcggctctggctgagatgcttcatgtacttggcgcgaaggttttggcagttgatccggtttaccatgctcaaaatgaagcttttttggattatacaaatcttgacgatgctttgaaaagggctgatgttatcactttccatacgcctttaaacaagcagaccaatggtatggccgacaaagatttttttcaaaaagttaaaaagaatgcatacattttaaactttgcccgcggagggattattcaaacttcggctttattggaaaatttgcgtagcggaaaattagctggagcagctctggatgttttgcccaatgaaaccgagtttatgggcaaaatccaaaatcctgacaaacttccggctgatgttcaggaattaatgtcgagggacaacgttttactgtcgccacacgtggctttttatacgaacctggccgttaaaaatatggttgaaatagctttaaacgatgcgattgccctttctcaaggcaaacatatcgagaatgaaattctttattaa

SEQ ID NO: 32 - protein, 325 aa, Oenococcus oeni
MYAVFEDEVPYIEEFANRTGHEIVQETDYLSEKNVDLAAGFDAVVTQQTVNIPDMVYSRLAELGIRQISIRQVGYDILNIDAIDKAGLRASNVAAYSPRAIAEYTLTQLLNLIRDNKRYYQATESGNWLWSAAPQGKEIHDLTVAVIGAGRIGSALAEMLHVLGAKVLAVDPVYHAQNEAFLDYTNLDDALKRADVITFHTPLNKQTNGMADKDFFQKVKKNAYILNFARGGIIQTSALLENLRSGKLAGAALDVLPNETEFMGKIQNPDKLPADVQELMSRDNVLLSPHVAFYTNLAVKNMVEIALNDAIALSQGKHIENEILY

SEQ ID NO: 33 - DNA, 939 bp, Bacillus coagulans
  CDS (1)..(939): Bacillus coagulans 36D1 gene Bcoa_0653 encoding lactate dehydrogenase
atgaaaaaggtcaatcgtattgcagtggttggaacgggtgcagttggtacaagttactgctacgccatgattaatcagggtgttgcagaagagcttgttttaatcgatattaacgaagcaaaagcagaaggggaagccatggacctgaaccacggcctgccatttgcgcctacgccgacccgcgtttggaaaggcgattattccgattgcggcactgccgatcttgttgtcattacggcaggttccccgcaaaaaccgggcgaaacaaggcttgatcttgttgccaaaaacgcaaaaatttttaaaggcatgattaagagcatcatggacagcggctttaacgggatttttcttgttgccagcaacccggttgacattttgacatatgtaacttggaaagagtccggcctgccgaaagaacatgttatcggttcgggcacagtgcttgactccgcgcgtctccgcaactctttaagcgcccacttcggaattgacccgcgcaatgtccatgccgcaattatcggcgaacacggcgacacggaacttccggtttggagccatacaacgatcggttatgacaccattgaaagctatctgcaaaagggaaccattgaccaaaaaacattagatgatatttttgtcaacacgagagatgcggcttaccatatcattgaacgaaaaggggccacattttacggcatcgggatgtctctgacccggatcacaagagcgatcctgaacaatgaaaacagtgttttgacagtctctgcctttttggaaggccagtacggaaacagcgatgtgtacattggtgttcctgccgttattaaccgccaaggcgtccgtgaagtggttgaaatcgagctgaacgacaaagaacaggaacaatttagccattctgttaaagtattaaaagaaacgatggcacctgtattgtaa

SEQ ID NO: 34 - protein, 312 aa, Bacillus coagulans
MKKVNRIAVVGTGAVGTSYCYAMINQGVAEELVLIDINEAKAEGEAMDLNHGLPFAPTPTRVWKGDYSDCGTADLVVITAGSPQKPGETRLDLVAKNAKIFKGMIKSIMDSGFNGIFLVASNPVDILTYVTWKESGLPKEHVIGSGTVLDSARLRNSLSAHFGIDPRNVHAAIIGEHGDTELPVWSHTTIGYDTIESYLQKGTIDQKTLDDIFVNTRDAAYHIIERKGATFYGIGMSLTRITRAILNNENSVLTVSAFLEGQYGNSDVYIGVPAVINRQGVREVVEIELNDKEQEQFSHSVKVLKETMAPVL

SEQ ID NO: 35 - DNA, 984 bp, Bacillus coagulans
  CDS (1)..(984): Bacillus coagulans 36D1 gene Bcoa_3093 encoding lactate dehydrogenase
  tRNA (1)..(3): Met is inserted by tRNA at start codon (GTG) instead of Val
gtgaaaaagacaaaattagtagttgcaggcgttgggcatgtcggttcgtatgtgcttgcaaatgcgatgaaactgggccttttttcggaaattgctgtattggataaaaagaaaggggtcgcatttggggaagcgctcgattggagacatgccacggcacttacatatatgccaaacacaagcgtgaaagcaggggattactcggaatgcgccgatgcggacgtcattatttgtgcagcagggccaagcgtgcttccgtcggaaaaagacgaaatgccggaccgggcagggcttgccaggacgaatgccgccgtggtgcgggaagtcatggcagggattacgaaatatacaaaagaagctgtcatcattttcattacaaatccgcttgatacgattgtctatattgctgaaaatgaatttggctattcgaaagggcggatattcggaacaggcaccatgcttgactcggcccgcctccgccagcttgttgccgaaaattacagcattgatccgaaaagtgtaacgggttatatgatgggcgaacacgggtttaccgcatttccggttttcagtcgtttaaatgtacagggattccgtgaaaaagaactggacagcgtgtttaaaggaaaagaaccgttggacagggaagctttcagacaaaaggtggtgaaaactgcttttgatgtcctgaatgggaaagggtggaccaatgccggtgttgctgaagctgcagtgacattggcaaaagccgtgatgctggatgaaaaaagcatttatccggtttcagccactttgcatggtcagtacggatataacggtgatgtcgctttaagcataccaagtgtcatcggccgtaacgggatcgaacaacagcttgaaattgaactcgatgaacaggaaacatcctggctgcacgaatcggcaaaatcgatccaaaatacaatggcatcggtaaaaaccggaaaatcgtatattcaggcttga

SEQ ID NO: 36 - protein, 327 aa, Bacillus coagulans
VKKTKLVVAGVGHVGSYVLANAMKLGLFSEIAVLDKKKGVAFGEALDWRHATALTYMPNTSVKAGDYSECADADVIICAAGPSVLPSEKDEMPDRAGLARTNAAVVREVMAGITKYTKEAVIIFITNPLDTIVYIAENEFGYSKGRIFGTGTMLDSARLRQLVAENYSIDPKSVTGYMMGEHGFTAFPVFSRLNVQGFREKELDSVFKGKEPLDREAFRQKVVKTAFDVLNGKGWTNAGVAEAAVTLAKAVMLDEKSIYPVSATLHGQYGYNGDVALSIPSVIGRNGIEQQLEIELDEQETSWLHESAKSIQNTMASVKTGKSYIQA

SEQ ID NO: 37 - DNA, 972 bp, Lactococcus lactis
  CDS (1)..(972): Lactococcus lactis subsp. cremoris llmg_1429 (ldhX) gene encoding lactate dehydrogenase
atgaaaattaataataaaaaagttgtaattgtgggagctggagcagtagggagtacttatgctcataatttggtcgttgatgatttagcagatgaaatcgccattatcaatactaataaaagtaaggcttcagccaatagtttggacttacttcatgctctgccttatttaaactcagctcctaaaaatatttatgcagcagactacgctgatgttgctgacgccgatatcgttgttttatccgctaatgctccgtcagctacctttggtaaaaatccagaccgtcttcaattactagaaaataatgttgagatgattcgtgatatcacgcgaaaaacaatggacgctggctttgacggtattttcttagtagcttcaaaccccgtagacgtacttgctcaagtagttgctgaagtttctggcttacctaaacaccggattattggaacaggaaccttgcttgaaacaagtcgaatgcgacaaattgtagctgagaaacttcaaattaaccctaaaagtattcatggttatgttttagctgaacatgggaaatctagttttgccgcttggtccaatgttactgttggggcaattccattaacaacttggcttgaaaaatatccaaatccagattttccgacatttgatgaaattgatcaagaaattcgtgaagttggtttagatattttcatgcaaaaaggaaatacttcatacggaatcgcagcttcattagcacgtttaacaagagcaattttccgaaatgagagcgtcattttacctgtttcggctcatttgacaggtcagtatggacaagctgaactatatacaggtagtccagcaataatcgaccggacaggtgttcgagccgttttagaattggaattaacagcagaagagcaaggtaaatttgaacattctgcagcactcttaaaagagaactttgaatccatcaaggaaaaatgcacattataa

SEQ ID NO: 38 - protein, 323 aa, Lactococcus lactis
MKINNKKVVIVGAGAVGSTYAHNLVVDDLADEIAIINTNKSKASANSLDLLHALPYLNSAPKNIYAADYADVADADIVVLSANAPSATFGKNPDRLQLLENNVEMIRDITRKTMDAGFDGIFLVASNPVDVLAQVVAEVSGLPKHRIIGTGTLLETSRMRQIVAEKLQINPKSIHGYVLAEHGKSSFAAWSNVTVGAIPLTTWLEKYPNPDFPTFDEIDQEIREVGLDIFMQKGNTSYGIAASLARLTRAIFRNESVILPVSAHLTGQYGQAELYTGSPAIIDRTGVRAVLELELTAEEQGKFEHSAALLKENFESIKEKCTL

SEQ ID NO: 39 - DNA, 945 bp, Lactococcus lactis
  CDS (1)..(945): Lactococcus lactis subsp. cremoris llmg_0392 (ldhB) gene encoding lactate dehydrogenase
atgaaaattacaagtagaaaagtagtcgtgattggaacaggatttgtcggaacaagtattgcttattcaatgattaatcaagggcttgtcaacgagttagttttgatagatgtcaatcaagataaagctgagggtgaggctttagatttattggatggcatatcttgggctcaagaaaatgttattgtaagagctggaaattacaaggattgtgaaaatgcagatattgtcgtgattacagctggtgtgaaccaaaaacccggacaatcacgtttagatttggtcaatacaaatgctaaaattatgcgctcaatcgttactcaagttatggattcaggttttgatggtatttttgtaattgcttctaatcctgtggatatccttacctatgtcgcttgggaaacttctggtcttgaccaatctagaattgtcggaactggtacaactttggataccacacgttttagaaaagaattggccacaaaactagaaattgaccctcggagtgttcacggatatattattggtgagcatggcgattctgaagttgcagtttggtcacacactacgataggtggaaaacctattcttgaatttattgttaaaaataaaaaaattggtcttgaagatttgtctaatttgtcaaataaggttaaaaatgctgcttatgaaattattgataaaaaacaagccacttattacggaattggaatgagcacagctagaattgtcaaagccatattaaataatgaacaagtcattctaccagtttctgcctatttgcgtggagaatatggtcaagaaggggtctttactggtgttccatctgtcgtaaatcaaaatggtgtcagagaaattattgaactcaatattgatgcttatgaaatgaaacaatttgaaaagtctgtcagtcaacttaaagaagtcatagaatctattaaataa

SEQ ID NO: 40 - protein, 314 aa, Lactococcus lactis
MKITSRKVVVIGTGFVGTSIAYSMINQGLVNELVLIDVNQDKAEGEALDLLDGISWAQENVIVRAGNYKDCENADIVVITAGVNQKPGQSRLDLVNTNAKIMRSIVTQVMDSGFDGIFVIASNPVDILTYVAWETSGLDQSRIVGTGTTLDTTRFRKELATKLEIDPRSVHGYIIGEHGDSEVAVWSHTTIGGKPILEFIVKNKKIGLEDLSNLSNKVKNAAYEIIDKKQATYYGIGMSTARIVKAILNNEQVILPVSAYLRGEYGQEGVFTGVPSVVNQNGVREIIELNIDAYEMKQFEKSVSQLKEVIESIK

SEQ ID NO: 41 - DNA, 981 bp, Lactococcus lactis
  CDS (1)..(981): Lactococcus lactis subsp. cremoris MG1363 gene llmg_0763 encoding phosphotransacetylase
atggaactttttgaatcactgaaacaaaagattagtggaaaagacttaaaaattgtctttcctgaaggaactgatacacgtattttaggtgctgcaaatcgtcttaatgccgatggattgattacacctgtctttattggaaatgtcaaagaagtcactgaaacattgatttcacgtggaattaacccagagggctttgagatttacgacccagaaaattgtggacgttttgaaacaatgacagaaacatttgttgaacgtcgcaagggtaaagttacagaagaacaagcaaaagaaattttgaaagacccaaattactttggaacaatgcttgtttatatgggaattgctgacggaatggtttctggtgcaattcattcaactgctgatactgttcgtccggctctccaaatcattaaaacaaaaccaggagtaaaatctgtttctggtgcctttattatggttcgcggtcgtgatgacaatgaaaaatatatttttggtgactgtgcaattaacatcaaacctgatgctccaacacttgctgatatcgccgtagcctcagccgaaactgcaagactctttgatattgaccctaaaattgcgatgctttcattctcaactaaaggttctggtaaatcagaagatgtggataaagtggttgaagcaacaacactagttaaagaaggtcaccctgaagttgaaattgatggggaacttcaatttgatgctgcctttgttccagctgttgcacgtcaaaaagcacctgattcagaagttgctggtcgtgcaaacgtctttatcttcccagacattcaatcagggaatattggatacaaaattgcgcaacgtctaggtaattttgaagccattggcccaatcttacaaggattgaatgcgcctgtttctgacctttcacgcggttgtaacgaagaagatgtttataaacttgcaatcatcacagctgctcaagctttaaaataa

SEQ ID NO: 42 - protein, 326 aa, Lactococcus lactis
MELFESLKQKISGKDLKIVFPEGTDTRILGAANRLNADGLITPVFIGNVKEVTETLISRGINPEGFEIYDPENCGRFETMTETFVERRKGKVTEEQAKEILKDPNYFGTMLVYMGIADGMVSGAIHSTADTVRPALQIIKTKPGVKSVSGAFIMVRGRDDNEKYIFGDCAINIKPDAPTLADIAVASAETARLFDIDPKIAMLSFSTKGSGKSEDVDKVVEATTLVKEGHPEVEIDGELQFDAAFVPAVARQKAPDSEVAGRANVFIFPDIQSGNIGYKIAQRLGNFEAIGPILQGLNAPVSDLSRGCNEEDVYKLAIITAAQALK

SEQ ID NO: 43 - DNA, 990 bp, Lactobacillus acidophilus
  CDS (1)..(990): Lactobacillus acidophilus NCFM gene LBA0704 encoding phosphotransacetylase
atgagcgtattttcattattcaaagaacaagttgaacaagctgatgaaaagaaaaaaattattttcccagaaagtaatgatcttagaatcttaactgcagtttccaatttaaataaggacaatattatcgatccaattttgattggtaaaagagaagatattgaaaaattggctgcagaaaataatttagatcttgatggtgtaaagatttatgatcaaaataattatgctggtttagatgaaatggctgaagcatttgttaaagctagaaaaaaggatactagtattgccgaagcaaaggctaagctagcaaaaggtaattactttggtactatgctagtaaagatgggaaaagctgatggaatggtatcaggtgctgcgcattcaactgctaatactgttttacctgctttacaattaattcatgctgctaaggggatgcatcgcgtatcgggtgctttcgtaatggaaaaaggcgatgaaagatatatttttgctgattgtgctattaatattgatccagatgaagaaactttggcagaaattggttatcaatccgctcaaactgctaaaatggcaggaatcgatcctaaggttgcatttttaagcttttcaactaaagggtcagctgaaggtcctatggtaaataaagttcatgacgctgctgcaatgttccaaaaagatcatccagaaatccctgcagatggtgaattacaatttgatgctgcatttgttccatcagttggtgaaaaaaaagcaccaggctctaaggtggcaggacatgctaatgtatttgtattcccagaacttcaatctggaaatatcgcttataagatggtgcaacgacttggtaactttactgcagtaggtcctattttacaaggtctagctgctccagttaatgacttatcacgtggttgtagtgaacaagatatttacgatttggctatcgttactgctgcacaagctttggcaaaggatgaaaagtaa

SEQ ID NO: 44 - protein, 329 aa, Lactobacillus acidophilus
MSVFSLFKEQVEQADEKKKIIFPESNDLRILTAVSNLNKDNIIDPILIGKREDIEKLAAENNLDLDGVKIYDQNNYAGLDEMAEAFVKARKKDTSIAEAKAKLAKGNYFGTMLVKMGKADGMVSGAAHSTANTVLPALQLIHAAKGMHRVSGAFVMEKGDERYIFADCAINIDPDEETLAEIGYQSAQTAKMAGIDPKVAFLSFSTKGSAEGPMVNKVHDAAAMFQKDHPEIPADGELQFDAAFVPSVGEKKAPGSKVAGHANVFVFPELQSGNIAYKMVQRLGNFTAVGPILQGLAAPVNDLSRGCSEQDIYDLAIVTAAQALAKDEK

SEQ ID NO: 45 - DNA, 990 bp, Lactobacillus delbrueckii
  CDS (1)..(990): Lactobacillus delbrueckii subsp. bulgaricus ATCC 11842 ; gene Ldb0643 encoding phosphotransacetylase
atggaagtttttgaacaattaaagcaattgattaagcaaaaagacaagcagcggatcgttttcccggaaggggaggacccgcggatcctgacggcggtcagccgcttaaagcaagatgacctggttgaaccgatcgtcctggggcaaaaggaccaggtcctggctttggcggaaaaagctggcttggacatgaccgggatcgaaatcgtggactaccaaaacagcccggactttggcgagatggtggaaaagttccacgaacggatggccgggggcaagcacttccagaccatggaagaagcggaagctgccttgcgcgacgccaacttctacgggaccatgctggtttacaccggccaggccgacgggatggtgtccggggcggctcactcaacggccgacacggtccggccagccctcttcattttgaagaccaagccggggatgcaccggatttccggttccttcatcatggaaagaggggaagaaaagtatgtctttgctgactgcgcgatttccatcgacccgaccagcgaccagctggttgaattcgcggcccagtcagctgctacggccaagatgatcgggattgaccccaaggtggccttcctcagcttctcaacccacggctcagccaagggcccgcaggtagacaaggtggcagaagcggccaagctgttcaaggaaaagtacccggacatcccggcagacggcgatttgcagtttgacgcggccttcgtgccggcagttgctgaacttaaggcacctggttcagccgttggcggcagggctaatgtctttatcttcccggaactgcagtcaggcaacattgcctacaagattacccagcgcctgggcaactttacggccatcgggccggttttgcaggggattgccaagccggtcaacgacctctcccggggctgcagcgctgatgacgtctacaagatcgggattttgacggcggctcaagccgttatggaagggtag

SEQ ID NO: 46 - protein, 329 aa, Lactobacillus delbrueckii
MEVFEQLKQLIKQKDKQRIVFPEGEDPRILTAVSRLKQDDLVEPIVLGQKDQVLALAEKAGLDMTGIEIVDYQNSPDFGEMVEKFHERMAGGKHFQTMEEAEAALRDANFYGTMLVYTGQADGMVSGAAHSTADTVRPALFILKTKPGMHRISGSFIMERGEEKYVFADCAISIDPTSDQLVEFAAQSAATAKMIGIDPKVAFLSFSTHGSAKGPQVDKVAEAAKLFKEKYPDIPADGDLQFDAAFVPAVAELKAPGSAVGGRANVFIFPELQSGNIAYKITQRLGNFTAIGPVLQGIAKPVNDLSRGCSADDVYKIGILTAAQAVMEG

SEQ ID NO: 47 - DNA, 978 bp, Lactobacillus casei
  CDS (1)..(978): Lactobacillus casei ATCC 334; gene LSEI_0996 encoding phosphotransacetylase
  tRNA (1)..(3): Met inserted by tRNA at start codon (TTG) instead of Leu
ttggacctatttgaaagtttaaaagacaagattaatggcaaaaatttacggattgtatttccggaaggcgaagatccgcgcgttttgggcgcagcagttcggctcgcagctgacgggttgatacaggccatcgtgctaggtaatcctgataagattcagacattggcagctgctaaaagctgggatctcagcaagttgacagttcgcgatccagccaacgatgaccttcacgctaaaatggttgcggcctttgttgagcgtcgcaaaggcaaggcaaccccagaacaggccgaaaaaattgttcaggatgccaattattttggtacgatgctggtttacatgaaggaagcagacggtatggtttcaggcgcagttcattcaacggctgacacagtccggcctgctttgcaaattatcaagacgcgtcctggcgtgcatttggtcagtggtgcgttcattatgcagcgtggccgtgacgaacgctacttattcgcggataacgccatcaacattgatccagatgctgatcagctggctgaaattgcggttgagtcagctaaaactgctgccttgtttgatatcgaaccaccgcgcgtcgccttgctgtcattctcaaccaaaggcagtgctaagggaccgcaagtagataaggtagttgaagccactaagaaagctcatgaactggcacctgatttggccttagacggtgagttacaatttgatgctgcttttgtcccaaccgttgccaagcaaaaagcccctgattccaaagtggctggggaagcgaatgttttcattttccctgaattacaatccggtaatattggttataagattgcgcaacgattcggcgggtttgaagccattggcccaattttgcaaggtttgaatcagccagtttctgatttgagtcgtggggcaaatgaagaggatgtctataagttggccattattacagcggcccagaccttgctatga

SEQ ID NO: 48 - protein, 325 aa, Lactobacillus casei
LDLFESLKDKINGKNLRIVFPEGEDPRVLGAAVRLAADGLIQAIVLGNPDKIQTLAAAKSWDLSKLTVRDPANDDLHAKMVAAFVERRKGKATPEQAEKIVQDANYFGTMLVYMKEADGMVSGAVHSTADTVRPALQIIKTRPGVHLVSGAFIMQRGRDERYLFADNAINIDPDADQLAEIAVESAKTAALFDIEPPRVALLSFSTKGSAKGPQVDKVVEATKKAHELAPDLALDGELQFDAAFVPTVAKQKAPDSKVAGEANVFIFPELQSGNIGYKIAQRFGGFEAIGPILQGLNQPVSDLSRGANEEDVYKLAIITAAQTLL

SEQ ID NO: 49 - DNA, 978 bp, Lactobacillus plantarum
  CDS (1)..(978): Lactobacillus plantarum WCFS1; gene lp_0807 encoding phosphotransacetylase
atggatttatttgagtcattagcacaaaaaattactggtaaagatcaaacaattgttttccctgaaggaactgaaccccgaattgtcggtgcggcagcgcgattagctgcagacggcttggttaagccgattgttttaggtgcaacggacaaagttcaggctgtggctaacgatttgaatgcggatttaacaggcgttcaagtccttgatcctgcgacatacccggctgaagataagcaagcaatgcttgatgccctcgttgaacggcggaaaggtaagaatacgccagaacaagcggctaaaatgctggaagatgaaaactactttggcacgatgctcgtttatatgggcaaagcggatgggatggtttcaggtgcaatccatccaactggtgatacggtacggccagcgttacaaattattaagaccaagcccggttcacaccgaatctcgggtgcatttatcatgcaaaagggtgaggaacgctacgtctttgctgactgtgccatcaatattgatcccgatgccgatacgttagcggaaattgccactcagagtgcggctactgctaaggtcttcgatattgacccgaaagttgcgatgctcagcttctcaactaagggttcggctaagggtgaaatggtcactaaagtgcaagaagcaacggccaaggcgcaagctgctgaaccggaattggctatcgatggtgaacttcaatttgacgcggccttcgttgaaaaagttggtttgcaaaaggctcctggttccaaagtagctggtcatgccaatgtctttgtatttccagagcttcagtctggtaatattggctataagattgcgcaacgatttggtcattttgaagcggtgggtcctgtcttgcaaggcctgaacaagccggtctccgacttgtcacgtggatgcagtgaagaagacgtttataaggttgcgattattacagcagcccaaggattagcttaa

SEQ ID NO: 50 - protein, 325 aa, Lactobacillus plantarum
MDLFESLAQKITGKDQTIVFPEGTEPRIVGAAARLAADGLVKPIVLGATDKVQAVANDLNADLTGVQVLDPATYPAEDKQAMLDALVERRKGKNTPEQAAKMLEDENYFGTMLVYMGKADGMVSGAIHPTGDTVRPALQIIKTKPGSHRISGAFIMQKGEERYVFADCAINIDPDADTLAEIATQSAATAKVFDIDPKVAMLSFSTKGSAKGEMVTKVQEATAKAQAAEPELAIDGELQFDAAFVEKVGLQKAPGSKVAGHANVFVFPELQSGNIGYKIAQRFGHFEAVGPVLQGLNKPVSDLSRGCSEEDVYKVAIITAAQGLA

SEQ ID NO: 51 - DNA, 972 bp, Pediococcus pentosaceus
  CDS (1)..(972): Pediococcus pentosaceus ATCC 25745; gene PEPE_0337 encoding phosphotransacetylase
atggatttatttgaaggtttgaagtcaaaaattaagggccaaaatagaacagttgtatttcccgaaggtgaagatgttcgagttttagctgccgctattcgactagctgatgatggcttagttcaatcaatcttactcggagtggaagctgatattaaagctactgctgaaaagcatggattaaaccttgaaaatgtgcaaattattgatccgagccaatatccggaagccgatcaaaaggaaatgattgataagttagttgaacgtcgtaagggtaaaaataccgaagaacaggttatcgaaatgctcaaagatgtaaattacttcggaactatgctagtttacttaggcaaggcggatgccatggtatctggagcagttcatgctactggtgatacagttcgccccgctcttcaaattatcaagactaaaccaggaatgcgtcgaattagtggagcatttatcatgcaacgcgaaaatgaacgctatgtctttggggatgcagctattaatattgatcccgatgcagaaacattggctgaaattgcgattcaaagtgcagaaactgctcgcctatttgacatcgaccctaaggttgcaatgttaagcttttcaactaaaggctcagcggctggaccaatggtagataaagttcgcgaagctactaaggttgcccaagaacgtgatccaaagaccccatatgatggtgaacttcagtttgatgcagcctttgttccagctgttggagcaagtaaggctccagattcagaaattgcaggacatgcgaatgtctttatcttccctgaacttcaatctggaaacattggttataagattgctcaacgtctaggcggttttgaagcaattggcccaatcttgcaaggacttaatgcaccggtttccgatttatctcgtggagctagtgaagaagatgtctataaagtagcaattattaccgcagctcaagtttag

SEQ ID NO: 52 - protein, 323 aa, Pediococcus pentosaceus
MDLFEGLKSKIKGQNRTVVFPEGEDVRVLAAAIRLADDGLVQSILLGVEADIKATAEKHGLNLENVQIIDPSQYPEADQKEMIDKLVERRKGKNTEEQVIEMLKDVNYFGTMLVYLGKADAMVSGAVHATGDTVRPALQIIKTKPGMRRISGAFIMQRENERYVFGDAAINIDPDAETLAEIAIQSAETARLFDIDPKVAMLSFSTKGSAAGPMVDKVREATKVAQERDPKTPYDGELQFDAAFVPAVGASKAPDSEIAGHANVFIFPELQSGNIGYKIAQRLGGFEAIGPILQGLNAPVSDLSRGASEEDVYKVAIITAAQV

SEQ ID NO: 53 - DNA, 981 bp, Leuconostoc mesenteroides
  CDS (1)..(981): Leuconostoc mesenteroides subsp. mesenteroides ATCC 8293; gene LEUM_1466 encoding phosphotransacetylase
atggaactttttgagcaattaaaaaataaaattaccggtcaaaataagacaattgtatttccggaaggtgaagatccccgtatccagggcgcagctatcagattagcagctgataatttgatcgagccaatattgttgggggatgcacaggaaatttcaaaaactgcccaagcccataactttgatttatcgaacatagagacgattgatcctgcttcatatgatgagaatgaattggcaaaattaaatgcgacacttgtcgaaaggcgcaaagggaaaacagacgctgagacagctgccaaatggctacaaaatgttaactattttgggacaatgctggtttatactggcaaagctgacggtatggtgtcaggcgcagtacatccaactggtgatacagttcgtcctgcgttgcagatcattaaaacggcgcctggctcatcacgtatttctggtgcattcattatgcaaaaaggcgaagagcgttatgtatttgcggacgctgccattaacattgacattgattcggacacgatggctgaaattgcaatacaatcagcccacacagctcaagtgtttgatattgaaccaaaagttgcgatgctttcattttcaactaagggctccgcgaaatcacccttggtcgataaggtggcaactgccactgcattggctaagaagttagcacctgaactggcggactcaattgatggagagttacagtttgatgctgcgtttgttgaatctgttgctgccgcaaaggcgcctgattcaaaagtcgcaggtaaagcgaacacatttattttcccaagtctagaagcaggtaatattggctataaaattgcacaacgcttgggtgggtttgaagcgattggacctattttacaaggcttagcaaaacctgtttctgatttatcgcgaggagccaatgaagaggatgtttataaagtagctattattactgctgcgcaagcattataa

SEQ ID NO: 54 - protein, 326 aa, Leuconostoc mesenteroides
MELFEQLKNKITGQNKTIVFPEGEDPRIQGAAIRLAADNLIEPILLGDAQEISKTAQAHNFDLSNIETIDPASYDENELAKLNATLVERRKGKTDAETAAKWLQNVNYFGTMLVYTGKADGMVSGAVHPTGDTVRPALQIIKTAPGSSRISGAFIMQKGEERYVFADAAINIDIDSDTMAEIAIQSAHTAQVFDIEPKVAMLSFSTKGSAKSPLVDKVATATALAKKLAPELADSIDGELQFDAAFVESVAAAKAPDSKVAGKANTFIFPSLEAGNIGYKIAQRLGGFEAIGPILQGLAKPVSDLSRGANEEDVYKVAIITAAQAL

SEQ ID NO: 55 - DNA, 984 bp, Streptococcus thermophilus
  CDS (1)..(984): Streptococcus thermophilus CNRZ1066; gene str1455 encoding phosphotransacetylase
atgcgtagtctatttggtgaacttcgtgaacatatttcagggaaaggagttaggattgttttccctgagggtaatgatgaccgtgtggttcgtgcagctgcccgtttgaaatttgaagaattagttgatcctattattttgggagaccctacagaggttcacaaaatcttgaacgatcttggttttgtagactttaactgtactattattaatcctgaaacctatgaaggcttcgaggaaatgaaggaaaaatttgttgaagtccgtaaagggaaagcgacaattgaagatgcagacaagttgcttcgtgatgtcaactattttggtgttatgctagttaaacttggtttagcagatggaatggtttcaggcgcgattcactcaacagcggataccgttcgtcctgctcttcaaattatcaaaacgaaacctggtatttcttgtacatcaggtgtcttccttatgaaccgtgaagataccaatcatcgcttgatgtttgccgactgtgctatcaatattgaaccaaattctcaagaattagcagaaattgcaattaatactgcagaaacagctaaagtttttggtattgatcctaaagtggcaatgcttagcttctcaacaaaaggttccgctaaatcaccaaaagttgacagggttgttcaagcaacaaatattgctaaagaaatgcgtcctgacttggctattgatggtgagttgcagtttgatgcagcctttgttccagcaacagcagctattaaggcaccagacagtgacgtagcaggacaagctaacgtctttgtcttcccatgtttacaggctggtaatattggttataagattgcccaacgtttgggaatgtttgaggctatcggaccaatcttacaaggtcttaataaaccagtaaacgatctttctcgtggtgctagtgctgaagatatttacaacttagctattatcacagctgctcaagctcttaataattaa

SEQ ID NO: 56 - protein, 327 aa, Streptococcus thermophilus
MRSLFGELREHISGKGVRIVFPEGNDDRVVRAAARLKFEELVDPIILGDPTEVHKILNDLGFVDFNCTIINPETYEGFEEMKEKFVEVRKGKATIEDADKLLRDVNYFGVMLVKLGLADGMVSGAIHSTADTVRPALQIIKTKPGISCTSGVFLMNREDTNHRLMFADCAINIEPNSQELAEIAINTAETAKVFGIDPKVAMLSFSTKGSAKSPKVDRVVQATNIAKEMRPDLAIDGELQFDAAFVPATAAIKAPDSDVAGQANVFVFPCLQAGNIGYKIAQRLGMFEAIGPILQGLNKPVNDLSRGASAEDIYNLAIITAAQALNN

SEQ ID NO: 57 - DNA, 990 bp, Oenococcus oeni
  CDS (1)..(990): Oenococcus oeni PSU-1; gene OEOE_1435 encoding phosphotransacetylase
atgacaaatttatttactgagttggctgataaaattaaggaaaaaaaactgagactcgtttttccggaaggcgatgattcaagagtccagggggcggccatccgtttaaaaaaagatggtttactaacgccgatattattgggagatcaggcagcggtcgaaaagacagctgcagataatcatttcgacctgagtggcatcaagcttatcgatccgcttaaattcgatgagaagcagttcaatcaattgatcgataaactggttacccgacgaaaagggaaaactgacccgcaaacggtagctatgtggctgcaggaagtaaattatttcggtaccatgcttgtttataccggcaaggccgacgcaatggtttccggtgctgttcatccaaccggcgatacggttcgtcccgctttgcagattatcaagacagctcctggtgtcagtcgtatttccggtgcttttattatgcaaaaaaatgatgaacgctatctgtttgccgattcggcaattaatatcgaacttgatgcacaaaccatggctgagattgccgttcaatcagcccatacggctaaaatatttggaatcgatccaaaagttgccatgctgagtttttcaaccaaaggatcggcaaaacatgcattggtcgataaagttgctcaggcaactaagctggcgcaggagatggcaccggatttagctgataatattgatggtgaattacaattcgatgcagcttttgtcgaatcggttgccaaattaaaagctccgggatcaaaagtggccggtcatgcgaacgttttcatttttccaagtttggaagccggaaatattggttataaaattgcccaacgcttgggtggatacgaagcaattggtcctattttgcaaggattggcagcacctgtttccgatctgtcgcgtggagcaagtcaagaagacgtatataaagttgcaattattactgctgctcaggctttaaaaaattag

SEQ ID NO: 58 - protein, 329 aa, Oenococcus oeni
MTNLFTELADKIKEKKLRLVFPEGDDSRVQGAAIRLKKDGLLTPILLGDQAAVEKTAADNHFDLSGIKLIDPLKFDEKQFNQLIDKLVTRRKGKTDPQTVAMWLQEVNYFGTMLVYTGKADAMVSGAVHPTGDTVRPALQIIKTAPGVSRISGAFIMQKNDERYLFADSAINIELDAQTMAEIAVQSAHTAKIFGIDPKVAMLSFSTKGSAKHALVDKVAQATKLAQEMAPDLADNIDGELQFDAAFVESVAKLKAPGSKVAGHANVFIFPSLEAGNIGYKIAQRLGGYEAIGPILQGLAAPVSDLSRGASQEDVYKVAIITAAQALKN

SEQ ID NO: 59 - DNA, 972 bp, Bacillus coagulans
  tRNA (1)..(3): Met inserted by tRNA at start codon (GTG) instead of val
  CDS (1)..(972): Bacillus coagulans 36D1; gene Bcoa_1406 encoding phosphotransacetylase
gtgagtacattttttgacgggataaaagaaaaagtgcggggacataacaaaacgattgtttttccggaaagttcggatgaaagggttttaacagccgtctcccggtttgcagccgatgaagtactccggccggttttaatcgggaacaaggataaaattcaggcaaaagcccaggccatcggtgtttctgtcaagggcgttacagtgtatgacccggaagactatgccggttttgaagaaatggtgcaggcttttgtggaacgaagaaaaggcaaagcgacggaggaagatgcgcgccaaattttaaaagacgtcaattatttcggcaccatgctcgtctatttgaaaaaagcggacggtctggtaagcggtgccatccattcaacggcagatacagtccggccggcattgcagatcattaaaacaaaaccgggcgtgaaaaaagtttctggcgttttcattatggtgaggggcgaagagaaatttgttttcgccgattgtgcaatcaatatttcaccggacagccaagacttggcggaaacggccgtggagagcgcgaaaactgcccggacatttgatattgaaccgaggattgcgatgttgagcttttcaacaagaggttccgcccattccgatgagacggagaaagtggcgcaggcagtccgattggctaaagagcgcgatccggagcttgtcatggacggcgagatgcagtttgatgccgcgtatgtgccggctgtggcgcaaaagaaagcgccggattccgtgattcaggggaatgcaactgtgtttatttttccaaatctggatgcggggaatatcggttataaaatggcccagcgtcttggcggttttgaagcagtcggtccgattttgcaagggctgaatgcgcctgtcaacgatttgtcgcgcggctgcaatgcggaagatgtttatcatttggctttgatttccgcagcacaaagtttgtaa

SEQ ID NO: 60 - protein, 323 aa, Bacillus coagulans
VSTFFDGIKEKVRGHNKTIVFPESSDERVLTAVSRFAADEVLRPVLIGNKDKIQAKAQAIGVSVKGVTVYDPEDYAGFEEMVQAFVERRKGKATEEDARQILKDVNYFGTMLVYLKKADGLVSGAIHSTADTVRPALQIIKTKPGVKKVSGVFIMVRGEEKFVFADCAINISPDSQDLAETAVESAKTARTFDIEPRIAMLSFSTRGSAHSDETEKVAQAVRLAKERDPELVMDGEMQFDAAYVPAVAQKKAPDSVIQGNATVFIFPNLDAGNIGYKMAQRLGGFEAVGPILQGLNAPVNDLSRGCNAEDVYHLALISAAQSL

SEQ ID NO: 61 - DNA, 2712 bp, Lactococcus lactis
  CDS (1)..(2712): Lactococcus lactis subsp. cremoris MG1363; gene llmg_2432 encoding alcohol dehydrogenase
atggcaactaaaaaagccgctccagctgcaaagaaagttttaagcgctgaagaaaaagccgcaaaattccaagaagctgtcgcttatactgatcaattagtcaaaaaagctcaagctgcagttcttaaatttgaaggatacacacaaactcaagttgatactattgttgctgcaatggctcttgcagcaagcaaacattctctggaactcgctcacgaagccgttaatgaaactggccgtggagttgttgaggacaaagatacaaaaaaccattttgcttctgaatctgtttataatgcaatcaaaaatgataaaacagttggcgttatcgctgaaaacaaagttgctggttctgttgaaatcgcaagcccccttggagtacttgctggtattgtcccaacaactaatccaacatcaacagccatctttaaatcattattaactgcaaagacacgtaatgctattgtctttgcctttcacccacaagcacaaaaatgctcaagccatgcggcaaaaattgtttatgatgctgcgattgaagctggtgcacctgaagactttattcaatggattgaagtacccagtcttgatatgacgactgctttgattcaaaatagaggaattgctacaattcttgcaactggtggtccaggtatggtcaatgccgcgcttaagtctggtaatccttcacttggtgtaggtgctggtaatggtgcagtttatgttgatgcaactgcaaatatcgatcgtgctgttgaagatcttttgctttcaaaacgttttgataacggaatgatttgtgcgactgaaaactctgcagttattgatgcatcaatctatgatgaatttgtcgctaaaatgcaagcgcaaggcgcttatatggttcctaaaaaagattacaaggcaattgaaagttttgttttcgttgaacgtgctggtgaaggttttggtgtaactggtcctgttgctggtcgttctggtcaatggattgctgaacaagctggtgttaacgtccctaaagataaagatgttcttctttttgaacttgataagaaaaatattggggaagctctttcttctgaaaaactttctcctttgctttcaatctacaaatcagaaacacgtgaagaaggaattgaaattgtacgtagcttacttgcttaccaaggagctggtcacaacgctgccattcaaatcggtgcaatggacgacccatttgtcaaagaatacggaattaaagtcgaagcttctcgtatcctcgttaaccaacctgactctatcggtggggtcggagatatttatactgatgcaatgcgtccatcattgacgctcggaactggttcatgggggaaaaattcactttcacacaatttgagtacatacgatctattgaatgttaaaacagtggctaaacgtcgtaatcgccctcaatgggttcgtttgccaaaagaaatttactacgaaaaaaatgcaatttcttacttacaagaattgccacacgtccacaaagctttcattgttgccgaccctggtatggttaaattcggtttcgttgataaagttttggaacaacttgctatccgcccaactcaagttgaaacaagcatttatggctcagtccaacctgacccaactttgagtgaagccattgcaatcgctcgtcaaatgaaacaatttgaacctgacactgtcatctgtcttggtggtggttctgctctcgatgctggtaagattggtcgtttgatttatgaatatgatgctcgtggtgaggctgacctttccgatgacgcaagtttgaaagagatcttccaagagttagctcaaaaatttgttgatattcgtaaacgtattatcaaattctaccacccacacaaagcacaaatggttgctatccctactacttctggtactggttctgaagtgactccatttgcggttatcactgatgatgaaactcacgttaaatatccacttgctgactatcaattgacacctcaagttgccattgttgaccctgagtttgttatgactgtaccaaaacgtactgtttcttggtctgggattgatgctatgtcacacgcgcttgaatcttatgtttctgtcatgtcttctgactatacaaaaccaatttcacttcaagccatcaaactcatctttgaaaacttgactgagtcttatcattatgacccagctcatccaaccaaagaaggtcaaaaagctcgcgaaaacatgcacaatgctgcaacactcgctggtatggccttcgccaatgctttccttggaattaaccactcacttgctcataaaattggtggtgaatttgggcttcctcatggtcttgccattgctatcgctatgccacatgtcattaaatttaacgctgtaacaggaaacgttaaacgtaccccttacccacgttatgaaacttatcgtgcgcaagaagactacgctgaaatttcacgcttcatgggatttgctggcaaagaagattcagatgaaaaagcggtcaaagctttggttgctgaacttaaaaaattgactgatagtattgatattaatatcaccctttcaggaaatggtgtagataaagctcatcttgaacgtgagcttgataaattggctgaccttgtttacgatgaccaatgtacacctgctaatccacgtcaaccaagaattgatgagattaaacaactcttgttagaccaatattaa

SEQ ID NO: 62 - protein, 903 aa, Lactococcus lactis
MATKKAAPAAKKVLSAEEKAAKFQEAVAYTDQLVKKAQAAVLKFEGYTQTQVDTIVAAMALAASKHSLELAHEAVNETGRGVVEDKDTKNHFASESVYNAIKNDKTVGVIAENKVAGSVEIASPLGVLAGIVPTTNPTSTAIFKSLLTAKTRNAIVFAFHPQAQKCSSHAAKIVYDAAIEAGAPEDFIQWIEVPSLDMTTALIQNRGIATILATGGPGMVNAALKSGNPSLGVGAGNGAVYVDATANIDRAVEDLLLSKRFDNGMICATENSAVIDASIYDEFVAKMQAQGAYMVPKKDYKAIESFVFVERAGEGFGVTGPVAGRSGQWIAEQAGVNVPKDKDVLLFELDKKNIGEALSSEKLSPLLSIYKSETREEGIEIVRSLLAYQGAGHNAAIQIGAMDDPFVKEYGIKVEASRILVNQPDSIGGVGDIYTDAMRPSLTLGTGSWGKNSLSHNLSTYDLLNVKTVAKRRNRPQWVRLPKEIYYEKNAISYLQELPHVHKAFIVADPGMVKFGFVDKVLEQLAIRPTQVETSIYGSVQPDPTLSEAIAIARQMKQFEPDTVICLGGGSALDAGKIGRLIYEYDARGEADLSDDASLKEIFQELAQKFVDIRKRIIKFYHPHKAQMVAIPTTSGTGSEVTPFAVITDDETHVKYPLADYQLTPQVAIVDPEFVMTVPKRTVSWSGIDAMSHALESYVSVMSSDYTKPISLQAIKLIFENLTESYHYDPAHPTKEGQKARENMHNAATLAGMAFANAFLGINHSLAHKIGGEFGLPHGLAIAIAMPHVIKFNAVTGNVKRTPYPRYETYRAQEDYAEISRFMGFAGKEDSDEKAVKALVAELKKLTDSIDINITLSGNGVDKAHLERELDKLADLVYDDQCTPANPRQPRIDEIKQLLLDQY

SEQ ID NO: 63 - DNA, 2631 bp, Lactobacillus acidophilus
  CDS (1)..(2631): Lactobacillus acidophilus NCFM; gene LBA0461 encoding alcohol dehydrogenase
atggctaaggcacaacaaaaagaaactcctactgaggaagaaaagaaaaagcaaatctacgacatggtagataatttagtaaaaagatcacatgttgcattagatgaaatggctaacttcacacaagaacaagttgataagatctgtgaagcagttgctactgcaggtgaacaaaacgcttacccactagctaaaatggctgttgaagaaactaagcgtggtgtagtagaagataagactactaaaaacatgtacgctagtgaaaatatttggaacagtttacgccacgaaaaaactgttggtgtaattgaagaagataaggaactaggattaactaagattgctgaacctaagggtgttattgccggtgttactccagtaactaacccaacttcaactgttattttcaaagcaatgcttgctctaaagacgagaaatactattatttttggtttccacccacaagctcaaaaatgttgtgtagaaactggtaaaattatccaagcggctgctgttgctgcaggtgcacctaaagatgcaattttatggattgaagagccaagtttggatgcaaccactgatttaatgaataatccaggtgttcaaactattttggccactggtggtccaggtatggttaaggctgcatattcatcaggtaaaccagctatcggtgttggtcctggtaatggtccttcatacattgaaaaatcagctaatatcggtcgtgctgtttatgatattgttttatcaaagaacttcgataacggtatggtttgtgcttctgaaaactcagtcgttgtagatgacgcaatttatgacaaggctgtagaagaatttaagaagtggggttgttacttcttaaataaagaagaaattaagaaatttgaagaacactttattgatccaagacgtggtactgttgcaggtcctgtagctggtaaatcagcttatgaaattgctaaactttgtggtgttgatgtaccaaagactactaaagtaattgttgctgaatacaaaggtgttggtagaaagttcccactttcagctgaaaaactttctcctgtctttactttatatcgcgctaagaatcatgacgatgcatttaagatttgttctgaacttcttgattatggtggtcgtggtcataccgcgggaattcactcaagcgatcaaaatataattgatgaatttgcaatgaagatggatgcttgtcgtattctagttaattcaccagctgcacttggtggtattggtgatctttacaacaatatgttgccttcacttacattaggtacaggttcatacggtgctaacacattttcacacaatattggtgccagagatttacttaatattaaaacggttgcgaaacgtcgcgacaatatgcaatgggtaaaagttccatcaaagacttactttgaacataatgctgctaattatttgcgtcacatgccaaatgttaaccgtttcttcattgttactgatgaaggcgtagctgaccaaggctttgctgacgaaatcactgatattatttctaagcgtcgtggtcaaaaagaatatgaagtatttaaggctgtaacgcttgatcctgatactgatgtaattaaagacggtgttcacagaatgaacatctttaagcctgacgtaattattgctattggtggtggctcagtaatggatgctgctaaagtaatgcgcttattctacgaaaatccagaaatgagctttgaagaagcttaccaaaagtttttggatatcagaaaaagagtagtccgcttccctaagattaatggtgttcaacttgtatgtattccaacaacttctggtactggttcagaagtttccccaattgcagttattagcgatactaaaacaggtatgaaacacactctttgtgattatgctttaactccagatgtttcaattgttgatgatcaatttgttcaaaatatgcctaaacgcttaattgcttggtcaggttttgaagcattaggtcatgctattgaaagctatgtttcaactatggccactgatttcactcgtggttggtcacttgaagctattaaagcgatctttgcaaacttgaagaagtcatacgatggtgatcttgatgcaagaaagcaaatgcatgatgctgcaactattgcaggtatggcatattcaaatgcattccttggtcttgaacactcaattgctcatactgttggctcaacctttgatattccaagcggagtaagtgatgcgattgctttaccgcaagttattcgctttaatgccaaacgtcctgaaaagctcgctatgtggcctcactactcagtatatcgtgctgaaagtgactatgctgatattgcaagagctatcggtttgactggttcaagtgatgctgaattagttgaaaaactggttcaaaagattattgaattggctcattctgttggtattaagatggcttataaggattatggcgttgacaagactgaatttgataagaaagttgatcaaatggcagttgaagcttatggtgatcaaaatactgtaactaatccatcagcaccacttattacacaaattgttgtcttaatgaaagattgctacgaaggcaaaggtatttcagacaatgataaataa

SEQ ID NO: 64 - protein, 876 aa, Lactobacillus acidophilus
MAKAQQKETPTEEEKKKQIYDMVDNLVKRSHVALDEMANFTQEQVDKICEAVATAGEQNAYPLAKMAVEETKRGVVEDKTTKNMYASENIWNSLRHEKTVGVIEEDKELGLTKIAEPKGVIAGVTPVTNPTSTVIFKAMLALKTRNTIIFGFHPQAQKCCVETGKIIQAAAVAAGAPKDAILWIEEPSLDATTDLMNNPGVQTILATGGPGMVKAAYSSGKPAIGVGPGNGPSYIEKSANIGRAVYDIVLSKNFDNGMVCASENSVVVDDAIYDKAVEEFKKWGCYFLNKEEIKKFEEHFIDPRRGTVAGPVAGKSAYEIAKLCGVDVPKTTKVIVAEYKGVGRKFPLSAEKLSPVFTLYRAKNHDDAFKICSELLDYGGRGHTAGIHSSDQNIIDEFAMKMDACRILVNSPAALGGIGDLYNNMLPSLTLGTGSYGANTFSHNIGARDLLNIKTVAKRRDNMQWVKVPSKTYFEHNAANYLRHMPNVNRFFIVTDEGVADQGFADEITDIISKRRGQKEYEVFKAVTLDPDTDVIKDGVHRMNIFKPDVIIAIGGGSVMDAAKVMRLFYENPEMSFEEAYQKFLDIRKRVVRFPKINGVQLVCIPTTSGTGSEVSPIAVISDTKTGMKHTLCDYALTPDVSIVDDQFVQNMPKRLIAWSGFEALGHAIESYVSTMATDFTRGWSLEAIKAIFANLKKSYDGDLDARKQMHDAATIAGMAYSNAFLGLEHSIAHTVGSTFDIPSGVSDAIALPQVIRFNAKRPEKLAMWPHYSVYRAESDYADIARAIGLTGSSDAELVEKLVQKIIELAHSVGIKMAYKDYGVDKTEFDKKVDQMAVEAYGDQNTVTNPSAPLITQIVVLMKDCYEGKGISDNDK

SEQ ID NO: 65 - DNA, 2607 bp, Lactobacillus casei
  CDS (1)..(2607): Lactobacillus casei ATCC 334; gene LSEI_0775 encoding alcohol dehydrogenase
atgttaaagaatactgcgaagactgctaccgaagttgatgtaaagagcatggttgacgaactggttgcgaatgcgcatgccgctttgaagatcatgaaaactttcgatcaagaaaagatcgatcacatcgttcatcggatggcaatcgctggtttggatcatcatatggaattggcaaaactcgctgttgacgagacgggccgtggcgtgtgggaagataaagcgatcaagaacatgttcgccaccgaagaaatttggcactcaatcaagaacaacaagaccgttggtgtgatcaacgaagacaaacaacgcggcttggtatcgattgctgaaccaattggtgtcattgctggtgtgacgccagtgacgaacccaacttcaacgaccatgttcaaatccgaaattgccattaaaacccgtaatccaatcattttcgctttccatccgggtgcccaaaagtcttcagcgcgtgcgttggaggtcatccgtgatgaagccgaaaaagcaggcttaccaaagggtgctttgcaatatatccctgtaccaagtatggatgcgactaaagctttgatggatcaccctggtatcgccacgattttggcaacgggcggtcctggcatggttaagtccgcctattcatcaggcaagccagcactgggtgttggggccgggaatgcgccagcttacattgaagccagcgctaacatcaaacaagctgtcaatgacttagtactttcaaagagttttgataatggtatgatttgtgcttccgaacaaggcgtgattattgattccagcatttacgatgatgtgaagaaggaatttgaagcccaaggtgcttacttcgttaaacagaaggacatgaagaagttcgaaagtaccgtcattaatcttgaaaagcaaagtgtcaatccgcgaatcgttggtcaaagtcctaagcagattgctgaatgggcggggatcacgattcctgataacaccacgattctgattgctgaattgaaaggtgttggcgaaaagtatccactgtctcgtgaaaaactaagtccagtactggccatggtcaaagcagatggtcatgaagatgccttcaagaaatgtgaaaccatgcttgatatcggcggtttgggtcatacggcagtcattcacacggctgatgatgagttagctttgaagtatgccgatgctatgcaagcttgccggattttgatcaacacaccatcttctgtcggcgggattggcgatctttacaacgaaatgattccaagtttgacactgggctgcggctcatacggtggtaactcaatttcgcataacgtggggacggtcgacttgcttaacatcaagacaatggcaaaacgccgcaataacatgcagtggatgaaattgccaccaaagatctattttgaaaagaattctgttcgttacctcgaacacatggaaggcatcaaacgggccttcatcgttgctgatcgaagcatggaaaagttaggcttcgtcaagatcgttgaagatgttctggctcggcgtgaaaatcctgttcaagttcagacttttgtcgatgttgaacctgatccatcaactgacacggtcttcaaggggaccgatatcatgcgctcatttggaccggataccgtcattgctatcggcggcgggtctgttatggatgctgccaaagggatgtgcttgttcaacgatgccggggatgctgatttcttcggcgctaagcagaaattcttggatatccggaagcggacttacaccttcccgaatctgaataagacaaagcttgtctgcattccaacaacttctgggactggttcagaagtaacaccttttgcggttatcaccgattccaaggctggcatcaagtatccgttagctgactatgcattgacgccagacatcgcgattgtggacagtcagttcatcgaatccgtaccgccagtggttgtcgctgattccggtttggatgtgctctgccacgcaaccgaaagttatgtttcgaccatggccactcattacacgaagggcctgagtttggaagcaatcaagttggtcttcgaaaatctggaaaagagttatcatggcgacgtcgaagcaaagtcaaagatgcatgatgcttcaaccattgccgggatggcctttgccaacgcactgttgggcattaaccacagcattgcgcataagattgggcaagcgttccatctgcctcacggtcgttgcatcgcgatcacgatgccacatgttattcgcttcaatgcgagtcaaccgaagaagcgcgctatctgggcgaagtatagctacttccgcgctaacgaagactacgcagaaatcgctcgctaccttggccttcctggcaagacaactgatgaattggttgaaagctatgtccaggccttcatcaagctggctcacagtgttggcgtcaagctcagtttgaaggaacaaggcgttaaaaaggctgatctcgacaagcaagttgatcgtcttgccgaactggcttatgaagacaactgcaccgttaccaacccgcaagaaccgttaatcggcgacttgaagcatatcatccttgatgaatatgaaggcaccgaatccactttctaa

SEQ ID NO: 66 - protein, 868 aa, Lactobacillus casei
MLKNTAKTATEVDVKSMVDELVANAHAALKIMKTFDQEKIDHIVHRMAIAGLDHHMELAKLAVDETGRGVWEDKAIKNMFATEEIWHSIKNNKTVGVINEDKQRGLVSIAEPIGVIAGVTPVTNPTSTTMFKSEIAIKTRNPIIFAFHPGAQKSSARALEVIRDEAEKAGLPKGALQYIPVPSMDATKALMDHPGIATILATGGPGMVKSAYSSGKPALGVGAGNAPAYIEASANIKQAVNDLVLSKSFDNGMICASEQGVIIDSSIYDDVKKEFEAQGAYFVKQKDMKKFESTVINLEKQSVNPRIVGQSPKQIAEWAGITIPDNTTILIAELKGVGEKYPLSREKLSPVLAMVKADGHEDAFKKCETMLDIGGLGHTAVIHTADDELALKYADAMQACRILINTPSSVGGIGDLYNEMIPSLTLGCGSYGGNSISHNVGTVDLLNIKTMAKRRNNMQWMKLPPKIYFEKNSVRYLEHMEGIKRAFIVADRSMEKLGFVKIVEDVLARRENPVQVQTFVDVEPDPSTDTVFKGTDIMRSFGPDTVIAIGGGSVMDAAKGMCLFNDAGDADFFGAKQKFLDIRKRTYTFPNLNKTKLVCIPTTSGTGSEVTPFAVITDSKAGIKYPLADYALTPDIAIVDSQFIESVPPVVVADSGLDVLCHATESYVSTMATHYTKGLSLEAIKLVFENLEKSYHGDVEAKSKMHDASTIAGMAFANALLGINHSIAHKIGQAFHLPHGRCIAITMPHVIRFNASQPKKRAIWAKYSYFRANEDYAEIARYLGLPGKTTDELVESYVQAFIKLAHSVGVKLSLKEQGVKKADLDKQVDRLAELAYEDNCTVTNPQEPLIGDLKHIILDEYEGTESTF

SEQ ID NO: 67 - DNA, 1194 bp, Lactobacillus casei
  CDS (1)..(1194): Lactobacillus casei ATCC 334; gene LSEI_2125 encoding alcohol dehydrogenase
  tRNA (1)..(3): Met inserted by tRNA at start codon (TTG) instead of Leu
ttgaagaattttcgatttcagaataaaaccgatattcgttttggtacaggccgattagatgttgagctgcacgacgcggtgagtcaatttggttcgcgtgttttgttcgtttatggtggccattccatcaaaaagtcaggcttatacgataccgtgactggactgctcgctgatttgcagttaaccgaattgccgggcattgagccgaacccgaagatcgattctatccgtgctggccaaaagctggcgaaggatcaggatattgatgttgttttggcagttggtggcggctccgtcattgatgctgcaaaagtcattgccagcgccaagttttacgaaggcgacccctgggatcttgtcaaagatcgtggtgtaactcgccggaaattgcagcaattgccagtcgtcgatattctgacattagcagcgaccggcagtgagatgaacagtggctccgtgatttcaaatcctgtcactcatgaaaagctcggcacagtcgggccaaacacaccagcggtttcatttttggatccgtcactgacctttactgtgccagccagacaaacggcggcgggatcaatggatattcttagtcatttgcttgaacaatattttgatcgttcaaaaaataatgacgccagcaaaggcatgattgaaggcttgatgcgcacagttattaagtgggcgcccattgcgttgaaacaacccgataactatgatgcccgcgccaacttaatgtggagcgcaacgatggcgctaaacggtatcgtgagtgttgctaacgaaaatggctggacagtgcatagccttgaacatgaactatcagcttattatgacattacccatggtgtcggccttggcattttaacaccgcgttggatgtcgtttattctgaccaaggacgcaacgacagcagaaatgttggcacaatttggtcgccatgtctgggatcttgaaggtgatgaatcttatgccgtggcgcaagcggctattgacgcgacctttgattggattagtcgcttagggttcccgatgaccttgcctgaagttgggatcaaggatactcagcattttgatgccatggcgactgctgctgttgccaatgctagtcttgaaacgcgcgcctatgtgccgttgtcggtagaagatgtcgaaacgatttatcaagcatccatgacagatgggctggcataa

SEQ ID NO: 68 - protein, 397 aa, Lactobacillus casei
LKNFRFQNKTDIRFGTGRLDVELHDAVSQFGSRVLFVYGGHSIKKSGLYDTVTGLLADLQLTELPGIEPNPKIDSIRAGQKLAKDQDIDVVLAVGGGSVIDAAKVIASAKFYEGDPWDLVKDRGVTRRKLQQLPVVDILTLAATGSEMNSGSVISNPVTHEKLGTVGPNTPAVSFLDPSLTFTVPARQTAAGSMDILSHLLEQYFDRSKNNDASKGMIEGLMRTVIKWAPIALKQPDNYDARANLMWSATMALNGIVSVANENGWTVHSLEHELSAYYDITHGVGLGILTPRWMSFILTKDATTAEMLAQFGRHVWDLEGDESYAVAQAAIDATFDWISRLGFPMTLPEVGIKDTQHFDAMATAAVANASLETRAYVPLSVEDVETIYQASMTDGLA

SEQ ID NO: 69 - DNA, 2604 bp, Lactobacillus plantarum
  CDS (1)..(2604): Lactobacillus plantarum WCFS1; gene lp_3662 encoding alcohol dehydrogenase
atgattaagacagaaaaaaatcaaacaagcaaagttactgacgaagttgatcaattagttcaacgttctaagaaagcattagctattttgaagtcatatacccaagcacaaatcgatgatttatgtgaaaaggttgcggttgccgcactggacaatcatatgaagttagccaagttggcggttgaggaaaccggtcggggtgttgttgaagataaggcgattaagaacatctacgccagcgaatatatctggaacagcatgcgccatgataagacagtcggcgtcattaaagaagacgatgaagagcaactaatggaaattgctgaaccagttggtatcgtagccggggttacacccgtaacgaatccaacttcaacaacggttttcaagacgttgatctcattaaaggggcgtaacacgatcgtctttggtttccatcctcaagctcagaaatgtagttctgcggctgctgatgtgatgcgggaagcgattaaagctgctggtggtccagctgatgctgttttatacatcgaacacccaagcattgaagcaaccgacgccttgatgcaccatacggatgttgcgacgatcttggctaccggtggtccaagtatggtgacagccgcttactcatcaggtaaaccagcactgggtgttggtcctggtaacggcccaacttatgttgaaaaaacagctgacattaagcaggcggttaacgacattgttttatcgaagacgtttgataatgggatgatttgtgcttctgaaaatagtgccattatcgacaaggaaatttatgctgaagttaaagctgaatttattcgtttgggttgctactacgttaaacctaaagatgtgcaagccttgagtgatgctgtcattgatcctaaccggcacacagttcgtggtccagttgctggtaaaacagcttatcaaatcgcacaaatggctggcttgaaagatgtcgctaaggactgccgggtcttgattgcggagattaatggtgtcggtatcaagtacccattatccggcgaaaaattatcaccagttctgaccgtttacaaagcggattcgcatgaagcggcgttcaaacgggctaatgaattacttcactatggtggcttaggccatactgctgggattcatacgaccgatgatgcattggtcaaagaatttggcttacaaatgccagcttgccggatcttagttaacacgccatcttcagttggtgggttaggtaacatctacaataacatggcgccttcattaactctgggcacgggatcatatggtggcaactcgatttctcataacgttacagatatggatctgattaatatcaaaactgtggcaaaacggcgtaataacatgcaatgggtcaaaatgcctcccaaagtctacttcgaacgcaattccgttcgctacttggaacacatggccgggattaagaaggttttcctagtttgtgatcctgggatggttgaatttggctatgctgatcgagtaacggcggtcttgaacaagcggactgatccggttgatatcgacatcttctcagaagttgaaccgaacccgtcgactgacacagtctacaaaggggttgcccggatgaaggcctttaagccagatacaatcattgcactcggtggtggttctgcaatggacgccgctaaggggatgtggctcttctatgaacacccagaagcttccttcttaggtgctaaacagaagttcctggatatccggaagcggacgtacaaagtaccagtatccgaaaaagtaacttatattgggattccaacgacttctgggactggttctgaagtaacgccatacgcggttattacggactccaagacgcacgtcaaatacccgattacggactacgcaatgcaaccagatattgcgatcgttgacccacaatttgttgaaacggtgccaaagcggacgacggcttggactggtctcgacgtaattacccacgcaaccgaagcatatgtttcaacgatggcttctgacttcacacgcggctggtcaattcaagccttgcaattagccttcaagtacttgaaagcttcttatgatggtgacaagatggctcgtgagaagatgcacaatgcctcaacgttagctgggatggcctttgccaatgccttcttgggtatcaaccactcgatcgcccataaattaggcggggaattcaacttaccacacggattggcaattgcgatcacttatccgcaagtcgttcgttacaatgctgaaatcccaacgaagttagcaatgtggccgaagtacaaccataatactgccttagccgactatgccaacattgcccgggccttagggttaccaggcaagaccgacgaagaactcaaagaaagcttagttaaagcttacattgacttggctcactcaatggatgtgaccttatccttgaaagctaaccgggtcgaaaagaagcactttgacgccaccgtcgacgaattagccgaactggcctatgaagaccaatgcacgaccgctaacccacgcgaacctttaatcagtgaattaaaggcgattattgaacgtgaatgggacggccaaggtactgaaaaatag

SEQ ID NO: 70 - protein, 867 aa, Lactobacillus plantarum
MIKTEKNQTSKVTDEVDQLVQRSKKALAILKSYTQAQIDDLCEKVAVAALDNHMKLAKLAVEETGRGVVEDKAIKNIYASEYIWNSMRHDKTVGVIKEDDEEQLMEIAEPVGIVAGVTPVTNPTSTTVFKTLISLKGRNTIVFGFHPQAQKCSSAAADVMREAIKAAGGPADAVLYIEHPSIEATDALMHHTDVATILATGGPSMVTAAYSSGKPALGVGPGNGPTYVEKTADIKQAVNDIVLSKTFDNGMICASENSAIIDKEIYAEVKAEFIRLGCYYVKPKDVQALSDAVIDPNRHTVRGPVAGKTAYQIAQMAGLKDVAKDCRVLIAEINGVGIKYPLSGEKLSPVLTVYKADSHEAAFKRANELLHYGGLGHTAGIHTTDDALVKEFGLQMPACRILVNTPSSVGGLGNIYNNMAPSLTLGTGSYGGNSISHNVTDMDLINIKTVAKRRNNMQWVKMPPKVYFERNSVRYLEHMAGIKKVFLVCDPGMVEFGYADRVTAVLNKRTDPVDIDIFSEVEPNPSTDTVYKGVARMKAFKPDTIIALGGGSAMDAAKGMWLFYEHPEASFLGAKQKFLDIRKRTYKVPVSEKVTYIGIPTTSGTGSEVTPYAVITDSKTHVKYPITDYAMQPDIAIVDPQFVETVPKRTTAWTGLDVITHATEAYVSTMASDFTRGWSIQALQLAFKYLKASYDGDKMAREKMHNASTLAGMAFANAFLGINHSIAHKLGGEFNLPHGLAIAITYPQVVRYNAEIPTKLAMWPKYNHNTALADYANIARALGLPGKTDEELKESLVKAYIDLAHSMDVTLSLKANRVEKKHFDATVDELAELAYEDQCTTANPREPLISELKAIIEREWDGQGTEK

SEQ ID NO: 71 - DNA, 2703 bp, Leuconostoc mesenteroides
  CDS (1)..(2703): Leuconostoc mesenteroides subsp. mesenteroides ATCC 8293; gene LEUM_0146 encoding alcohol dehydrogenase
atggcagaagcaattgcaaagaaacccgcaaaaagggttttgacccctgaagaaaaagcggaattacaaacacaagctgagaagatgactgaggtattgattgaaaaatcacaaaaggcattgtctgaattttcaacattttcgcaagaacaagttgataaaattgttgcagctatggccttggcaggttctgagaattcacttctgttagcccatgctgctcacgacgagactggacgtggggttgtggaagataaggatacgaaaaatcgtttcgcctcagaatcagtttataacgctattaagtttgataagactgtgggtgttattagtgaagacaagattcaaggtaaggtagaattagcagccccacttggtattttggctggaatcgtcccaacgacaaatccaacgtcgacaactattttcaaatcaatgttgacagcaaagacacgtaacacaattatctttgctttccatccccaggctcaaaaagcatcggttcttgctgcaaaaattgtttatgatgctgctgttaaagcaggcgcaccggaaaactttatccaatggattgaaaagccttcactttatgcaacaagtgcgctgatacaaaatcctcacattgcttcaattctagctactggtgggccatcaatggttaatgcagctttgaagtcaggaaatccatccatgggtgtcggtgctggaaacggtgcagtttatattgatgcaactgttgacacagatcgtgccgtgtccgatttgttgttatcaaagcgtttcgataatggcatgatttgtgccacagaaaactcagccgttattcaagcaccaatctatgacgaaattttaactaagttacaagaacaaggtgcataccttgttcctaagaaagactacaaaaaaattgctgattatgtatttaagcctaacgcagagggatttggtattgctggtcctgttgctggtatgtcaggacgttggattgctgagcaagcaggcgtaaagattcctgatggtaaagatgtacttttgttcgaattagatcagaagaacataggtgaagcgttatcttctgaaaagttatcgccattactttcaatttataaagttgagaagcgtgaagaagctattgagactgttcaatccttgttaaactatcaaggcgcggggcacaacgcagcaattcaaattggttcacaagatgatccattcattaaagagtatgctgacgctattggtgcatcacgtattttggttaaccaacctgactcaatcggtggtgttggagatatttacacagatgctatgcgtccatcgttgacacttggtaccggatcatgggggaagaattcattgtctcataacttatcaacatacgacttacttaatattaagaccgtggctcgccgccgtaatcgtcctcaatgggttcgtttacctaaggaagtttactacgaagccaatgccattacttacttacaagacttgcctactataaaccgtgcatttattgtcgctgatcctggtatggttcagttcggatttgttggcagagtactaggtcaacttgagttacgtcaagaacaggttgaaacaaatatctatggttcagttaagcctgacccaactttgtcacaagctgttgaaattgctcgccaaatggcagacttcaaaccagatacagttattttacttggcggtggttcggcacttgacgctggtaaaattggtcggttcttgtacgaatactcgacacgccatgaaggaattttagaagatgacgaggcgattaaagatctattcttagaactacaacaaaagtttatggatattcgtaagcgaatcgttaagttttaccacgcacgtttgacacaaatggttgcgattccaacaacttcaggtactggatcagaagtcacaccatttgccgttattacagatgatgaaacacatgtaaagtatccactagccgattatgaattgacaccggaagttgctattgttgatccagaatttgttatgaccgtaccacaacacacggtatcttggtcaggattagatgctttgtcacatgctttggaatcgtatgtctcagtgatggcttctgaattcacacgtccttgggcattacaagctattaagttgatttttgataacttaacaaattcatacaattatgatcctaaacacccaactaaggaaggtcagaatgcacgcacaaagatgcactatgcgtcaacattggctggtatgtcatttgcgaatgccttcttgggacttaaccactcactagcacacaaaactggtggagaattcggactacctcacggtatggcaatcgctattgcaatgccacatgtgattaagtttaatgcggtaacaggaaatgtaaagcgcacaccatacccacgttacgaaacctatacagcacaaaaagattatgctgatattgcacgttacttaggtttgaaaggtgaaacagatgctgaattggtcgatgtattgattgcagaaatcaagaagttggctgcatcagtgggtgtcaatcaaacactatctggcaacggtgtttcaaagcatgactttgatacaaagttagaaaagatgattgacttagtttacaatgaccaatgcacgccgggaaaccctcgccaaccaagcttggcagaaattcgtcaattgttgaaagatcagttttaa

SEQ ID NO: 72 - protein, 900 aa, Leuconostoc mesenteroides
MAEAIAKKPAKRVLTPEEKAELQTQAEKMTEVLIEKSQKALSEFSTFSQEQVDKIVAAMALAGSENSLLLAHAAHDETGRGVVEDKDTKNRFASESVYNAIKFDKTVGVISEDKIQGKVELAAPLGILAGIVPTTNPTSTTIFKSMLTAKTRNTIIFAFHPQAQKASVLAAKIVYDAAVKAGAPENFIQWIEKPSLYATSALIQNPHIASILATGGPSMVNAALKSGNPSMGVGAGNGAVYIDATVDTDRAVSDLLLSKRFDNGMICATENSAVIQAPIYDEILTKLQEQGAYLVPKKDYKKIADYVFKPNAEGFGIAGPVAGMSGRWIAEQAGVKIPDGKDVLLFELDQKNIGEALSSEKLSPLLSIYKVEKREEAIETVQSLLNYQGAGHNAAIQIGSQDDPFIKEYADAIGASRILVNQPDSIGGVGDIYTDAMRPSLTLGTGSWGKNSLSHNLSTYDLLNIKTVARRRNRPQWVRLPKEVYYEANAITYLQDLPTINRAFIVADPGMVQFGFVGRVLGQLELRQEQVETNIYGSVKPDPTLSQAVEIARQMADFKPDTVILLGGGSALDAGKIGRFLYEYSTRHEGILEDDEAIKDLFLELQQKFMDIRKRIVKFYHARLTQMVAIPTTSGTGSEVTPFAVITDDETHVKYPLADYELTPEVAIVDPEFVMTVPQHTVSWSGLDALSHALESYVSVMASEFTRPWALQAIKLIFDNLTNSYNYDPKHPTKEGQNARTKMHYASTLAGMSFANAFLGLNHSLAHKTGGEFGLPHGMAIAIAMPHVIKFNAVTGNVKRTPYPRYETYTAQKDYADIARYLGLKGETDAELVDVLIAEIKKLAASVGVNQTLSGNGVSKHDFDTKLEKMIDLVYNDQCTPGNPRQPSLAEIRQLLKDQF

SEQ ID NO: 73 - DNA, 927 bp, Streptococcus thermophilus
  CDS (1)..(927): Streptococcus thermophilus CNRZ1066; gene str0936 encoding alcohol dehydrogenase
  tRNA (1)..(3): Met inserted by tRNA at start codon (GTG) instead of Val
gtggatctgacctttggagttaccgtaatccggaaatggaagagggacatagaaaatagtggtcatgaggctatcggaattgtcgaagaagttggtgagcaagttactacggtaaaaccaggagactttgtcattgcacctttcacacatgggtgtggtcagtgtgatgcttgtcgagctggttttgacggctcttgtgataaccactcaggtaataactggtctgagggcgtgcaggctgagtatatccgtttccactttgccaactgggcccttgtcaaaattcttgggcaaccctcagattatagcgaaggcatgctcaagtcacttttgacacttgctgatgttatgccgacgggttaccatgctgcgcgtgtggctaatgtacaaaagggtgataaggttgtcgttatcggagatggggctgttggtcaatgtgctgtcattgcatctaagatgcgtggagcttctcaaattgtccttatgagccgtcacgaagatcgtcaaaagatggctttggagtcaggtgcgactgcggttgtagctgagcgtggcgaaaaaggcattgacaaagtccgtgaagtcctcgggggaggagcagatgcagctctcgaatgtgtcggtactgaagcagcagtagaacaagctcttggtgtcctccataacggtggtcgtatgggatttgtcggtgtgcctcattataataaccgttcccttggttcgacctttgcccaaaatattttagtggcgggaggggtagcctctgtcacaacctatgataaacaagttttgttaaaagctgttcttgatggcgacatcaatccaggccgagtctttacgacaagttataaattggaagatattaatcaggcttataaagatatggatgaacgcaagtctattaaatctatgattgttttagattag

SEQ ID NO: 74 - protein, 308 aa, Streptococcus thermophilus
VDLTFGVTVIRKWKRDIENSGHEAIGIVEEVGEQVTTVKPGDFVIAPFTHGCGQCDACRAGFDGSCDNHSGNNWSEGVQAEYIRFHFANWALVKILGQPSDYSEGMLKSLLTLADVMPTGYHAARVANVQKGDKVVVIGDGAVGQCAVIASKMRGASQIVLMSRHEDRQKMALESGATAVVAERGEKGIDKVREVLGGGADAALECVGTEAAVEQALGVLHNGGRMGFVGVPHYNNRSLGSTFAQNILVAGGVASVTTYDKQVLLKAVLDGDINPGRVFTTSYKLEDINQAYKDMDERKSIKSMIVLD

SEQ ID NO: 75 - DNA, 2697 bp, Oenococcus oeni
  CDS (1)..(2697): Oenococcus oeni PSU-1; gene OEOE_1248 encoding alcohol dehydrogenase
atggcagttgaaacgaagaagattactaaaaagatactgacttccgacgaacttaaagacaaaaaggagacagcaaaaaaatacattggagaattagtcgataaatcaagacaggcattattagaattctctcaatatagtcaatcacaagttgacaaaattgttgccgcaatggctttagccggttccgaacattctttggaattagcacactttgctcgaaatgaaactggaagaggagttgttgaagacaaagatacgaaaaatcgttttgcctcggaatctgtatataacacgattaaaaatgacaaaacagttggcgtaattgatgaagacccaatcactggcaaagttcagttagctgcaccacttgggattttagccggaattgttccaacgacaaatccaacgtctaccacgatttttaaatcattgttaaccgctaagacacgtaatacgatcatttttgcttttcacccgcaagcacaaaaatcgtctgtagcggctgcgaaaattgtttaccaggcggctagaaaagctggagcaccgaaagatttcattcagtggatcgaaacgccttccttggaaaatacgactgctttgatgcaaaatcctgaaatagcttcaattttggctactggaggcccttctatggttcatgccgcattaacatcgggaaacccttcaatgggtgttggggcaggaaatggagctgttttcattgaccatacggctcgtgttcgtcgagcggcagaagacttgttgctatcaaaacgttttgataatggaatgatttgcgctacagagaattccgcagttatcgaagcttcggtttatgacgaatttatgaaattaatgcaggaaaagggtacttatctcgttccgaaggatgattacaaaaagatcgctgattttgttttcagcgataaacatgcagttaatggtccggttgccgggatgagtggccgttggattgctgaacatgccggtgttactttgccgaagggcaaagatgttttgttgtttgagttggatcaagacgacattggcgaaaaactttcctcggaaaaacttagccccttgctttcggtctataaagctgctgatcgtaaagaagcgataaaagttgttcaaagacttttgaactatcaaggagctggtcataatgcggcaatccagattggcgctcaagacgatccctttgttaaagaatatgccgatgcagtttctgcatctagaattttggttaaccagcccgattcaataggtggagtcggcgatatttatacagatgcattgcgtccaagtatgacgcttggtactggttcatgggggaagaattcattgtcacataatttatcaacttacgatttgttgaatgtcaaaacggttgcccgcagacgtaatcgtcctcaatgggttcgtcttccaaaagatatctactatgagaaaaatgccattacctatctgcaggaattaccgaacgtcaatcgggctttcgttattaccgatcattcgttggtcaaatatggattcgtcgatcggattcttgagcaattcgaattgcgtcccgaccaggttaaaacaagtatttacagttctgttcaaccggatccttatttgagtcaggctgttgaaattgcaaaacaaatgcaggagttcgaaccggatacagtaattgctcttggaggaggttcctctttggatgttgccaagatttcccgttttctttatgaatattctcaagaaccggaccacattggttttttggaaaacattgacgatattaaagaattgtttaaaggattgcagcaaaagtttatggatattcggaaacggattgttaaattcgaacatcagaatttgactcaactggttgctattccaacaacttcgggtaccggttcggaagtaacaccgttttcggttatcaccgatgatgaaactcacgttaaataccctttggccgattatgaattaactccgcaagttgcaatcgttgatcctgaattggttatgactgtaccaaaacggaccctggcttggtctggactggacgccttatctcactcgcttgaatcctacgtgtcggttatgagttctgaatttacacggccgtgggctttacaggcaatcaaactgatttttgaaaacatcgttgattcctataattacgatccaaagcacccaactaatcggggcgccgaggcacgtgaaaaaatgcattatgctgcaactttggccggcatgtctttcggaaacgccttcttgggtataaaccattcactggcccataaaaccggtggagagttcggtcttcctcatggtttggcaatcagcatcgcgatgccacatgtaattcgatttaatgcggttaccggaaacgtcaaacggactccttttcctcgttatgaggtttatacagcccaaaaagattatgccgatattgcgcgccatattggcctcagcggtaagaacgatgccgaattggttgaaaaacttatcgccaaaatcaaggaattaaccgatgccttggatgtcaatattactttgagcggcaatggagtcgataaaaagagctttgaacattctttggatcaactggttgatcttgtttacgatgaccagtgcacgccaggaaatccgcgacaaccgaatttagctgaaattaggcagttattgatcgaccaattttaa

SEQ ID NO: 76 - protein, 898 aa, Oenococcus oeni
MAVETKKITKKILTSDELKDKKETAKKYIGELVDKSRQALLEFSQYSQSQVDKIVAAMALAGSEHSLELAHFARNETGRGVVEDKDTKNRFASESVYNTIKNDKTVGVIDEDPITGKVQLAAPLGILAGIVPTTNPTSTTIFKSLLTAKTRNTIIFAFHPQAQKSSVAAAKIVYQAARKAGAPKDFIQWIETPSLENTTALMQNPEIASILATGGPSMVHAALTSGNPSMGVGAGNGAVFIDHTARVRRAAEDLLLSKRFDNGMICATENSAVIEASVYDEFMKLMQEKGTYLVPKDDYKKIADFVFSDKHAVNGPVAGMSGRWIAEHAGVTLPKGKDVLLFELDQDDIGEKLSSEKLSPLLSVYKAADRKEAIKVVQRLLNYQGAGHNAAIQIGAQDDPFVKEYADAVSASRILVNQPDSIGGVGDIYTDALRPSMTLGTGSWGKNSLSHNLSTYDLLNVKTVARRRNRPQWVRLPKDIYYEKNAITYLQELPNVNRAFVITDHSLVKYGFVDRILEQFELRPDQVKTSIYSSVQPDPYLSQAVEIAKQMQEFEPDTVIALGGGSSLDVAKISRFLYEYSQEPDHIGFLENIDDIKELFKGLQQKFMDIRKRIVKFEHQNLTQLVAIPTTSGTGSEVTPFSVITDDETHVKYPLADYELTPQVAIVDPELVMTVPKRTLAWSGLDALSHSLESYVSVMSSEFTRPWALQAIKLIFENIVDSYNYDPKHPTNRGAEAREKMHYAATLAGMSFGNAFLGINHSLAHKTGGEFGLPHGLAISIAMPHVIRFNAVTGNVKRTPFPRYEVYTAQKDYADIARHIGLSGKNDAELVEKLIAKIKELTDALDVNITLSGNGVDKKSFEHSLDQLVDLVYDDQCTPGNPRQPNLAEIRQLLIDQF

SEQ ID NO: 77 - DNA, 2613 bp, Bacillus coagulans
  CDS (1)..(2613): Bacillus coagulans 36D1; gene Bcoa_0619 encoding alcohol dehydrogenase
atggcaatcgatgaaaaagttgttggcaaatctgcgcaaatcacggaaatggtcgattccctcgtcgcaaaagggcaaaaagcactgcgtgaatttatggaactggatcaggcacaagtggacaatatcgtaaaacaaatggcacttgccgggttggaacagcatatggtgctcgccaaaatggccgtggaagaaacagggcgcggcgtatatgaagacaaaatgacaaaaaacttgtttgcaacagaatacatctatcacaacattaaatataacaaaacagtcggcatcattgacgaaaacaataatgaagggattgttaaatttgcggaaccggtcggcgtgattgccggggtcactcctgtaacaaacccgacatcaaccacgatgtttaaagcattaattgccattaaaacacgcaacccaattattttcgcgttccatccgtccgcacaaaaatgcagcagccatgccgcaaaagtgatgcttgacgctgctgtgaaagccggcgccccggaaaactgcatccaatggatcgaaaaaccggccattgaagcgacacagcaattgatgaaccattcgggtatttcactgattcttgcaacgggcggttccggcatggtaaagtccgcatattcgagcgggaaacctgcgcttggcgtcgggccgggaaacgtcccttgctatattgaaaaatctgctgatatcaaacgcgccgtcaatgatttgattttgtccaaaacgtttgacaacggcatgatctgtgcgtcggaacaggctgtcatcatcgacaaagaaatttatgacaatgtgaaaaacgaattgattgcaaatcaatgctacttcctgaatgaagcggagaagaaaaaggttgagaaaaccgtcatcaatgaaaagacgcaatccgtcaacagcgccattgtcggcaaaccggcctatgaaattgcgaaaatggcaggcgtaaatgttccggaagataccaaaattttaatcgccgaacttacaggtgtcggcccggactacccgctttccagagaaaaattaagcccggttttggcctgttacaaagccaacagcacaaaacaaggtttcgaatttgcagaagccatgctcgaattcggcggacttggtcactcggctgtgatccattcgacaaacgatgaagtcattgaacaatacggcttaaaaatgaaagccggccggatcattgtcaactccccgtcttcgcaaggggcaatcggcgacatttacaatgcctacatgccttctttgacactcggctgcggcacgtttggcggcaactcggtgtccacaaacgtcggtgtcatcaatctttacaatgtaaaaacaatggcgaaacggagagtcaatatgcaatggtttaaaattccaccgcgcgtgtattttgaaaagaactcggtccaatacctggaaaaaatgccggatatctcaagtgcctttattgtcaccgatccggatatggtcaggcttggattcgttgagaaagtactgtattacttgcggaaacgcccggactatgtgcactgtgaaattttctcagaagtcgaaccggatccgtctattgaaacggttcggaaaggtgccgcagctatggcaagcttccagccggatgtcattatcgcgcttggcggcgggtcggcgatggatgctgcaaaaggcatgtggctgttctatgagcatccggaagttgatttcaacgaattgaagcaaaaatttatggacatccggaaacgggttgccaaattcccgaaactgggtgaaaaagcgcagctggtctgcattccgaccacatcgggcaccggttctgaagtgacttctttcgccgtcatttcggataaaaagaacaacacaaaatatccgctcgcagactatgaactgacaccggatgttgcgatcattgacccgcagtttgttatgaccgttccgaaatccgtaacagccgataccgggatggacgttttgacccatgcgattgaagcctatgtatccaacatggccaacgactatacggacggcctggcaattaaggccatccagcttgttcacgaatacttgccgaaagcatacgcaaatggcaatgatgcactggcacgcgaaaaaatgcacaatgcatcgactttggcagggatggcgttctcgaatgcattccttggcatcaaccattcacttgcccataaaatcggcgctgaattccatattccgcacggccgggctaacgcgattctgttgccgcatgtcatccgctataacgcgcaaaagccgaaaaaatttgcggcattcccgaaatacgaacattttattgcggatcaacgctacgcggaaatcgcgcgcgtacttggtcttccggcaagcacgacagaagaaggtgtcgaaagcctttgccaggaaatcatccggatgtgcaaactgttcaatattccgcttagcctaaaagcagccggcgtgaaccgggcggactttgaaaaacgggttgccattattgcagaccgcgcatttgaagaccaatgcacacctgcaaaccctaaattgccgcttgtcagcgaattggaagacattttacgcaaagcttttgaaggcgttgaagcaaaataa

SEQ ID NO: 78 - protein, 870 aa, Bacillus coagulans
MAIDEKVVGKSAQITEMVDSLVAKGQKALREFMELDQAQVDNIVKQMALAGLEQHMVLAKMAVEETGRGVYEDKMTKNLFATEYIYHNIKYNKTVGIIDENNNEGIVKFAEPVGVIAGVTPVTNPTSTTMFKALIAIKTRNPIIFAFHPSAQKCSSHAAKVMLDAAVKAGAPENCIQWIEKPAIEATQQLMNHSGISLILATGGSGMVKSAYSSGKPALGVGPGNVPCYIEKSADIKRAVNDLILSKTFDNGMICASEQAVIIDKEIYDNVKNELIANQCYFLNEAEKKKVEKTVINEKTQSVNSAIVGKPAYEIAKMAGVNVPEDTKILIAELTGVGPDYPLSREKLSPVLACYKANSTKQGFEFAEAMLEFGGLGHSAVIHSTNDEVIEQYGLKMKAGRIIVNSPSSQGAIGDIYNAYMPSLTLGCGTFGGNSVSTNVGVINLYNVKTMAKRRVNMQWFKIPPRVYFEKNSVQYLEKMPDISSAFIVTDPDMVRLGFVEKVLYYLRKRPDYVHCEIFSEVEPDPSIETVRKGAAAMASFQPDVIIALGGGSAMDAAKGMWLFYEHPEVDFNELKQKFMDIRKRVAKFPKLGEKAQLVCIPTTSGTGSEVTSFAVISDKKNNTKYPLADYELTPDVAIIDPQFVMTVPKSVTADTGMDVLTHAIEAYVSNMANDYTDGLAIKAIQLVHEYLPKAYANGNDALAREKMHNASTLAGMAFSNAFLGINHSLAHKIGAEFHIPHGRANAILLPHVIRYNAQKPKKFAAFPKYEHFIADQRYAEIARVLGLPASTTEEGVESLCQEIIRMCKLFNIPLSLKAAGVNRADFEKRVAIIADRAFEDQCTPANPKLPLVSELEDILRKAFEGVEAK

SEQ ID NO: 79 - DNA, 1707 bp, Zymomonas mobilis
  CDS (1)..(1707): codon-optimised pdc gene encoding pyruvate decarboxylase (Z. mobilis)
atgagttatacagtaggaacatatcttgctgaacgattggtacaaattggacttaaacatcattttgcagttgctggagattataatttagttttacttgataatttgttacttaataaaaatatggaacaagtatattgttgtaatgaacttaattgtggattttcagctgaaggttatgctcgtgcaaaaggagctgctgctgctgttgtaacatatagtgttggtgctttatcagcatttgatgctattggtggagcttatgcagaaaatcttcctgttattttgatttctggagcaccaaataataatgatcatgctgctggtcatgttttacatcatgctcttggtaaaacagattatcattatcaattagaaatggcaaaaaatattacagctgctgctgaagctatttatactcctgaagaagctccagcaaaaattgatcatgttattaaaactgcacttagagaaaagaaacctgtatatcttgaaattgcatgtaatattgcttcaatgccatgtgcagctccaggtcctgcttctgcattatttaatgatgaagcatcagatgaagcatcattaaatgcagctgttgaagaaacacttaaatttattgcaaatcgtgataaagtagctgttttggtaggatctaaacttagagcagctggtgctgaagaagcagctgttaaatttgctgatgcattaggtggagctgtagcaactatggcagctgcaaaatcatttttccctgaagaaaatccacattatattggaacatcttggggtgaagttagttatcctggagtagaaaaaactatgaaagaagctgatgcagttattgctcttgcaccagtatttaatgattatagtacaactggatggactgatattccagatcctaaaaaacttgttttggcagaacctcgaagtgttgtagttaatggtattcgttttccatcagttcatcttaaagattatttgacacgacttgcacaaaaagtatcaaagaaaacaggagctttagatttctttaaaagtcttaatgctggtgaattgaaaaaagctgcaccagcagatcctagtgctccacttgttaatgcagaaattgctcgtcaagtagaagcattacttacacctaatacaactgttattgctgaaactggagattcatggtttaatgcacaaagaatgaaacttccaaatggtgctcgtgttgaatatgaaatgcaatggggacatattggttggtcagttcctgctgcatttggatatgcagtaggtgctccagaacgtagaaatattttgatggttggtgatggatcttttcaattaacagcacaagaagttgctcaaatggttagattaaaacttcctgttattatttttcttattaataattatggatatactattgaagtaatgattcatgatggtccatataataatattaaaaattgggattatgctggacttatggaagtttttaatggtaatggtggatatgattctggtgcaggaaaaggtcttaaagctaaaacaggtggagaattggctgaagcaattaaagttgctttagcaaatacagatggacctactcttattgaatgttttattggtcgagaagattgtactgaagaattagttaaatggggaaaaagagttgctgctgctaatagtagaaaacctgtaaataaattgttgtaa

SEQ ID NO: 80 - protein, 568 aa, Zymomonas mobilis
MSYTVGTYLAERLVQIGLKHHFAVAGDYNLVLLDNLLLNKNMEQVYCCNELNCGFSAEGYARAKGAAAAVVTYSVGALSAFDAIGGAYAENLPVILISGAPNNNDHAAGHVLHHALGKTDYHYQLEMAKNITAAAEAIYTPEEAPAKIDHVIKTALREKKPVYLEIACNIASMPCAAPGPASALFNDEASDEASLNAAVEETLKFIANRDKVAVLVGSKLRAAGAEEAAVKFADALGGAVATMAAAKSFFPEENPHYIGTSWGEVSYPGVEKTMKEADAVIALAPVFNDYSTTGWTDIPDPKKLVLAEPRSVVVNGIRFPSVHLKDYLTRLAQKVSKKTGALDFFKSLNAGELKKAAPADPSAPLVNAEIARQVEALLTPNTTVIAETGDSWFNAQRMKLPNGARVEYEMQWGHIGWSVPAAFGYAVGAPERRNILMVGDGSFQLTAQEVAQMVRLKLPVIIFLINNYGYTIEVMIHDGPYNNIKNWDYAGLMEVFNGNGGYDSGAGKGLKAKTGGELAEAIKVALANTDGPTLIECFIGREDCTEELVKWGKRVAAANSRKPVNKLL

SEQ ID NO: 81 - DNA, 1152 bp, Zymomonas mobilis
  CDS (1)..(1152): Codon optimised adhB gene encoding alcohol dehydrogenase (Z. mobilis)
atggcaagtagtacattttatattccttttgttaatgaaatgggtgaaggtagtttagaaaaagcaattaaagatttgaatggtagtggatttaaaaatgctcttattgttagtgatgcatttatgaataaatcaggtgttgtaaaacaagttgctgatttacttaaagcacaaggaattaatagtgctgtttatgatggtgtaatgccaaatcctacagttacagctgttttagaaggattgaaaattcttaaagataataatagtgattttgttatttcattaggtggaggttctcctcatgattgtgctaaagcaattgctttagttgctactaatggaggtgaagtaaaagattatgaaggaattgataaatctaaaaaaccagcattgcctcttatgagtattaatacaactgctggtactgcatcagaaatgacacgtttttgtattattactgatgaagttagacatgtaaaaatggctattgttgatcgacatgtaacaccaatggtttcagtaaatgatcctttgcttatggttggaatgccaaaaggtttaacagctgcaactggaatggatgcacttactcatgcttttgaagcatattcatctacagctgcaactcctattacagatgcatgtgctttgaaagctgcatctatgattgctaaaaatcttaaaacagcatgtgataatggtaaagatatgccagctcgtgaagcaatggcttatgcacaatttcttgctggaatggcatttaataatgcatctttgggttatgttcatgctatggcacatcaattgggaggttattataatcttcctcatggagtttgtaatgctgtacttttgccacatgttcttgcttataatgcaagtgttgtagcaggtcgtttaaaagatgttggagtagctatgggtttagatattgcaaatcttggagataaagaaggtgctgaagcaactattcaagctgttagagatttagctgcatctattggtattccagctaatcttacagaattgggtgctaaaaaagaagatgttccattacttgctgatcatgcacttaaagatgcttgtgcattgactaatccacgtcaaggagatcaaaaagaagtagaagaattgtttttatcagcattttaa

SEQ ID NO: 82 - protein, 383 aa, Zymomonas mobilis
MASSTFYIPFVNEMGEGSLEKAIKDLNGSGFKNALIVSDAFMNKSGVVKQVADLLKAQGINSAVYDGVMPNPTVTAVLEGLKILKDNNSDFVISLGGGSPHDCAKAIALVATNGGEVKDYEGIDKSKKPALPLMSINTTAGTASEMTRFCIITDEVRHVKMAIVDRHVTPMVSVNDPLLMVGMPKGLTAATGMDALTHAFEAYSSTAATPITDACALKAASMIAKNLKTACDNGKDMPAREAMAYAQFLAGMAFNNASLGYVHAMAHQLGGYYNLPHGVCNAVLLPHVLAYNASVVAGRLKDVGVAMGLDIANLGDKEGAEATIQAVRDLAASIGIPANLTELGAKKEDVPLLADHALKDACALTNPRQGDQKEVEELFLSAF

SEQ ID NO: 83 - DNA, 1707 bp, Lactococcus lactis
  CDS (1)..(1707): lacE gene encoding phosphotransferase system EIICB component
atgcataaactcattgaacttattgagaaagggaaaccattctttgagaaaatctctcgaaacatctatcttcgtgctattcgtgatggatttattgctggtatgccagtcatccttttctcatctatctttatccttattgcctatgtcccaaatgcttggggattccactggtcaaaagacattgagaccttcttgatgactccttatagctattcgatgggtatcctagcattctttgttggtggtactaccgctaaagctttgacggactcaaagaaccgtgacctacctgcgaccaatcagattaacttcttatctacgatgctagcgtctatggtagggttccttttgatggcagctgagcctgcaaaagaaggaggcttcttgacagccttcatgggaacaaaaggccttttgacagcatttatcgcagcctttgtgacagttaatgtttataaagtctgtgtaaaaaataatgttaccattcgtatgcctgaagatgttccaccaaatatctctcaagtatttaaggacttgattccgttcaccgtatcagttgttctcctatatggacttgaactccttgttaagggaactcttggtgtaactgttgcagaatcaatcggtacccttattgctcctcttttctcagctgcagatggttaccttggaattacacttatctttggtgcttatgccttcttctggtttgttggtattcacggtccttcaattgtcgaaccagcaatcgctgctatcacttatgccaatatcgatgtcaacttgcatcttatccaagctggacaacatgcagataaagttatcacttctggtactcaaatgtttattgctaccatgggtggaacaggagctacattgattgttccattcttgttcatgtggatttgtaaatcagatcgtaaccgtgccatcggacgtgcctcagttgttccaacattctttggggttaatgagccaatcttatttggtgcaccaatcgtattgaatccgattttctttgtaccgttcattttcgctccgattgtcaacgtttggatctttaaattctttgttgacacattgaacatgaactcattctctgccaaccttccatgggttactcctggtccgttgggaattgtactcggtactaacttccaagttctatcatttatcttagccggactcttggtagttgttgatactatcatttattacccatttgttaaggtatacgatgaacaaattcttgaagaagaacgttctggtaaaacaaacgatgctcttaaagaaaaagtagctgcaaacttcaatactgctaaagcagatgccgttcttggaaaagcagacgttgctaaagaagatgtagcagcaaacaataatatcacaaaagaaactaatgtccttgttctttgtgcaggtggaggtacaagtggcttgcttgccaatgccttgaacaaagcagcagcagaatacaatgttccagtcaaggcggcagcgggtggttacggtgctcaccgtgaaatgttgccagagtttgacttggtaatcttggctccacaggtcgcttcaaacttcgatgatatgaaggctgaaacagataaattgggcattaaacttgtaaaaactgaaggagctcaatatatcaaattgacacgtgacggacaaggtgctcttgcatttgttcaacaacagtttgattaa

SEQ ID NO: 84 - protein, 568 aa, Lactococcus lactis
MHKLIELIEKGKPFFEKISRNIYLRAIRDGFIAGMPVILFSSIFILIAYVPNAWGFHWSKDIETFLMTPYSYSMGILAFFVGGTTAKALTDSKNRDLPATNQINFLSTMLASMVGFLLMAAEPAKEGGFLTAFMGTKGLLTAFIAAFVTVNVYKVCVKNNVTIRMPEDVPPNISQVFKDLIPFTVSVVLLYGLELLVKGTLGVTVAESIGTLIAPLFSAADGYLGITLIFGAYAFFWFVGIHGPSIVEPAIAAITYANIDVNLHLIQAGQHADKVITSGTQMFIATMGGTGATLIVPFLFMWICKSDRNRAIGRASVVPTFFGVNEPILFGAPIVLNPIFFVPFIFAPIVNVWIFKFFVDTLNMNSFSANLPWVTPGPLGIVLGTNFQVLSFILAGLLVVVDTIIYYPFVKVYDEQILEEERSGKTNDALKEKVAANFNTAKADAVLGKADVAKEDVAANNNITKETNVLVLCAGGGTSGLLANALNKAAAEYNVPVKAAAGGYGAHREMLPEFDLVILAPQVASNFDDMKAETDKLGIKLVKTEGAQYIKLTRDGQGALAFVQQQFD

SEQ ID NO: 85 - DNA, 318 bp, Lactococcus lactis
  CDS (1)..(318): lacF gene encoding phosphotransferase system EIIA component
atgaacagagaagagatgactctcttagggtttgaaattgttgcttatgctggagatgctcgctctaagcttttagaagcgcttaaagcggctgaaaatggtgatttcgctaaggcagatagtcttgtagtagaagcaggaagctgtattgcagaggctcacagttctcagacaggtatgttggctcgagaagcttctggggaggaacttccatacagtgttactatgatgcatggtcaggatcacttgatgactacgatcttattaaaagatgtgattcatcacctcatcgaactttataaaagaggagcaaagtaa

SEQ ID NO: 86 - protein, 105 aa, Lactococcus lactis
MNREEMTLLGFEIVAYAGDARSKLLEALKAAENGDFAKADSLVVEAGSCIAEAHSSQTGMLAREASGEELPYSVTMMHGQDHLMTTILLKDVIHHLIELYKRGAK

SEQ ID NO: 87 - DNA, 1407 bp, Lactococcus lactis
  CDS (1)..(1407): lacG gene encoding phospho-beta-D-galactosidase
atgactaaaacacttcctaaagattttatttttggtggtgctacagctgcttatcaagcagaaggtgcaactcacacagatggtaagggcccagtagcatgggataagtatcttgaagacaattattggtatacagcagaacctgcaagtgatttttaccataaatacccagttgatttagaattagctgaagaatatggtgttaatggtattcgtatttctattgcctggtctcgtattttcccaacgggttacggtgaagtaaatgaaaaaggtgttgaattctaccataaactatttgctgaatgccacaagcgtcatgttgaaccatttgtgactttgcatcattttgacacaccagaagctcttcattcaaatggagatttcttaaaccgcgaaaatatagaacactttatagattatgccgctttctgttttgaagagtttccagaagtaaactactggacaactttcaatgaaattggcccaattggtgatggtcaatacttggttggtaaattccctccaggcattaaatatgatcttgccaaagtcttccaatcacaccacaacatgatggtctctcatgcacgtgctgtaaaattgtataaggataaaggttataaaggtgaaattggtgttgttcatgctttaccaactaaatacccttatgatccggaaaatccagcagatgttcgtgctgctgaacttgaagacatcatccataacaaatttatcttggatgcaacctatcttgggcactattcagataaaacgatggaaggtgtcaaccatatcctagctgagaatggtggagaacttgatcttcgtgatgaagacttccaagctcttgacgcagctaaagatttgaatgatttccttggtatcaactattacatgagtgattggatgcaagcttttgatggtgagactgaaatcattcacaatggtaagggtgaaaaaggaagctctaagtaccaaattaagggtgttggtcgtcgagtagctcctgactatgttccgcgcacagactgggattggattatttatcctgaaggcttgtatgaccaaatcatgcgagtgaaaaatgattatccgaattacaagaagatttacatcactgaaaacggtctcggatacaaagatgagtttgtagataatactgtttatgatgatggtcgtattgattacgtgaagcaacacttggaagttttatcagacgcgattgcggatggtgcaaatgttaaaggttacttcatttggtcacttatggacgttttctcatggtcaaatggttatgaaaaacgttatggattgttctatgtagactttgatacgcaagaacgctatcctaagaaatcagcacattggtataagaaattagcagaaactcaagtgatagagtaa

SEQ ID NO: 88 - protein, 468 aa, Lactococcus lactis
MTKTLPKDFIFGGATAAYQAEGATHTDGKGPVAWDKYLEDNYWYTAEPASDFYHKYPVDLELAEEYGVNGIRISIAWSRIFPTGYGEVNEKGVEFYHKLFAECHKRHVEPFVTLHHFDTPEALHSNGDFLNRENIEHFIDYAAFCFEEFPEVNYWTTFNEIGPIGDGQYLVGKFPPGIKYDLAKVFQSHHNMMVSHARAVKLYKDKGYKGEIGVVHALPTKYPYDPENPADVRAAELEDIIHNKFILDATYLGHYSDKTMEGVNHILAENGGELDLRDEDFQALDAAKDLNDFLGINYYMSDWMQAFDGETEIIHNGKGEKGSSKYQIKGVGRRVAPDYVPRTDWDWIIYPEGLYDQIMRVKNDYPNYKKIYITENGLGYKDEFVDNTVYDDGRIDYVKQHLEVLSDAIADGANVKGYFIWSLMDVFSWSNGYEKRYGLFYVDFDTQERYPKKSAHWYKKLAETQVIE

SEQ ID NO: 89 - DNA, 426 bp, Lactococcus lactis
  CDS (1)..(426): lacA gene encoding galactose-6-phosphate isomerase subunit (1)
atggctattgttgttggtgcagatctcaaaggtacgagattaaaagatgttgtgaaaaattttcttgtagaagaaggttttgaggtaattgatgtgactaaggacggacaagattttgttgatgttaccttggcagttgcctctgaagtaaataaagatgagcaaaacttaggtattgtaattgatgcttatggagctggtccattcatggtagcaactaaaattaaaggtatggttgcagcagaagtttcagatgaacgctcagcttatatgacacgtggacataacaatgcacgaatgattacggttggcgctgaaatcgtcggtgatgagcttgctaaaaacatcgctaaagctttcgtcaatggtaaatatgatggcggacgtcatcaagtccgagtagatatgcttaacaagatgtgttaa

SEQ ID NO: 90 - protein, 141 aa, Lactococcus lactis
MAIVVGADLKGTRLKDVVKNFLVEEGFEVIDVTKDGQDFVDVTLAVASEVNKDEQNLGIVIDAYGAGPFMVATKIKGMVAAEVSDERSAYMTRGHNNARMITVGAEIVGDELAKNIAKAFVNGKYDGGRHQVRVDMLNKMC

SEQ ID NO: 91 - DNA, 516 bp, Lactococcus lactis
  CDS (1)..(516): lacB gene encoding galactose-6-phosphate isomerase subunit (2)
atgagaatcgcaattggatgtgaccacattgtcacagatgtaaaaatggcagtatcagaattcttgaaatcaaaaggatatgaggtccttgatttcgggacatatgaccacgtacgtactcactaccctatctatggtaaaaaagtcggtgaagcagtagtaagtggtcaagcagacttgggagtatgtatctgtggtacgggtgtcggcattaacaatgcagtcaataaagttccaggtgttcgctcagcgttggttcgtgatatgacgtcagctctatatgctaaggaagaacttaatgcgaatgttatcggttttggtggaatgattactggtggtcttcttatgaatgacatcattgaagctttcattgaagctgagtacaaaccaacagaagaaaataaaaaattgattgcaaaaatcgaacacgttgaaacacataatgcacatcaagcagatgaggaattctttacagaattccttgaaaaatgggaccgtggtgagtaccacgattaa

SEQ ID NO: 92 - protein, 171 aa, Lactococcus lactis
MRIAIGCDHIVTDVKMAVSEFLKSKGYEVLDFGTYDHVRTHYPIYGKKVGEAVVSGQADLGVCICGTGVGINNAVNKVPGVRSALVRDMTSALYAKEELNANVIGFGGMITGGLLMNDIIEAFIEAEYKPTEENKKLIAKIEHVETHNAHQADEEFFTEFLEKWDRGEYHD

SEQ ID NO: 93 - DNA, 933 bp, Lactococcus lactis
  CDS (1)..(933): lacC gene encoding D-tagatose-6-phosphate kinase
atgattctgacagtcacactaaatccttcagtagacatctcttatcctctagaaacattgaaaattgatacagtgaatagggtgaaagatgttagtaagacagctggtggaaaaggcttaaatgttactagagtcttgtatgaatctggtgataaagttactgctacaggtttcttaggtggtaaaattggtgaatttattgaaagcgaattagaacaatctcctgttagtccagctttttataaaatctcaggaaacactagaaactgtattgctattttacacgagggtaaccaaacagaaattttggaacaaggaccaacaatttctcatgaagaagcagaaggctttcttgaccactatagcaatcttattaagcaatcagaagttgtcacaatttcaggaagtttgccttcaggacttcccaacgattattatgaaaaacttatccaacttgcttcggatgaaggagtagcagtcgttttggattgttcaggtgcaccacttgaaacagttctaaaatcgtcggctaaacctactgccatcaaacctaataatgaggaactttcacagttgcttggaaaagaagtaacaaaagatattgaagaactcaaagacgttcttaaagagtcactttttagtggtatcgaatggatagtggtctcattaggtcgaaacggtgcctttgcaaaacatggtgatgttttctataaggtagatattcctgatatcccagttgttaatccagtcggatcaggggactcaacggtagctggtattgcatcagctttaaatagtaaaaaaagtgacgctgacttattaaaacatgcgatgacattgggtatgttaaatgctcaagaaacaatgacagggcatgtgaatatgactaattacgaaacactaaactcacaaattggagtaaaagaggtataa

SEQ ID NO: 94 - protein, 310 aa, Lactococcus lactis
MILTVTLNPSVDISYPLETLKIDTVNRVKDVSKTAGGKGLNVTRVLYESGDKVTATGFLGGKIGEFIESELEQSPVSPAFYKISGNTRNCIAILHEGNQTEILEQGPTISHEEAEGFLDHYSNLIKQSEVVTISGSLPSGLPNDYYEKLIQLASDEGVAVVLDCSGAPLETVLKSSAKPTAIKPNNEELSQLLGKEVTKDIEELKDVLKESLFSGIEWIVVSLGRNGAFAKHGDVFYKVDIPDIPVVNPVGSGDSTVAGIASALNSKKSDADLLKHAMTLGMLNAQETMTGHVNMTNYETLNSQIGVKEV

SEQ ID NO: 95 - DNA, 981 bp, Lactococcus lactis
  CDS (1)..(981): lacD gene encoding tagatose 1,6-diphosphate aldolase
atggtacttacagaacagaaacgcaaatctttggaaaaactttcagataaaaacggttttatctcagctttggcatttgaccaacgtggtgctttgaaacgtttgatggcacagtatcaagatacagaaccaactgtagctcaaatggaagaactaaaggttttggttgctgatgaacttacaaagtacgcttcttcaatgttgcttgaccctgaatatggacttccagcaacaaaagcattggataaagaagcaggtcttcttcttgcctatgaaaaaacaggttatgatacatcaagtacaaaacgtttgcctgactgcttggatgtttggtctgcaaagcgtatcaaagagcaaggcgctgatgccgttaagttcttgctttactatgatgtagacagctcagatgaacttaaccaacaaaaacaagcttatattgaacgtgttggttctgaatgtgtcgctgaagatattcctttcttcttggaaatccttgcttatgatgaagaaatctcagatgcaggttccgttgaatatgcaaaagtgaaacctcgcaaagtcattgaagcgatgaaagttttctctgatccacgctttaacattgatgttcttaaagtagaagtaccagttaacgttaaatatgtcgaaggttttgctgatggtgaagtggtttacagtaaagctgaagctgctgacttctttaaagcacaagaagaagcgactaaccttccatacatctacctaagtgcaggtgtatctgctaaattgttccaagaaacattgcaatttgctcacgactcaggtgccaagtttaatggtgtgctttgtggacgtgctacttgggcaggatctgtagaaccttacatcaaagaaggtgaaaaagcagcacgtgaatggttgcgtactactggattcgaaaatattgatgaacttaacaaagttcttgttaaaacagctagtccatggactgataaagtatag

SEQ ID NO: 96 - protein, 326 aa, Lactococcus lactis
MVLTEQKRKSLEKLSDKNGFISALAFDQRGALKRLMAQYQDTEPTVAQMEELKVLVADELTKYASSMLLDPEYGLPATKALDKEAGLLLAYEKTGYDTSSTKRLPDCLDVWSAKRIKEQGADAVKFLLYYDVDSSDELNQQKQAYIERVGSECVAEDIPFFLEILAYDEEISDAGSVEYAKVKPRKVIEAMKVFSDPRFNIDVLKVEVPVNVKYVEGFADGEVVYSKAEAADFFKAQEEATNLPYIYLSAGVSAKLFQETLQFAHDSGAKFNGVLCGRATWAGSVEPYIKEGEKAAREWLRTTGFENIDELNKVLVKTASPWTDKV

SEQ ID NO: 97 - DNA, 768 bp, Lactococcus lactis
  CDS (1)..(768): lacR gene encoding a lactose transport regulator
atgaacaaaaaacgacgattagaaaaaattttagatatgttaaagattgatgggaccataaccataaaagaaataatagatgaactagatatttccgatatgacagcccgtagagaccttgatgctctagaagctgatggacttttaacacgtactcatggtggtgcacaattgctttcctctaaaaagccacttgaaaagacacatatcgagaagaaaagtctaaatacaaaagaaaaaattgacattgctaaaaaagcctgctctttaatcaaagatggcgatactatttttattggacccggaactacacttgtacaactggcattagaattgaaaggtcgtaaaggttataaaattcgtgtcattacaaatagtctccctgtgttcttgattctaaatgatagcgaaaccattgatttattgcttcttggcggtgaatatagagaaataactggagcttttgtaggttcaatggcttcgacaaatttaaaagcaatgagatttgccaaagcttttgttagtgcaaatgctgttacccataattctattgctacatatagtgacaaggaaggtgtgattcaacaacttgccctaaacaatgctgtagaaaaattcttattagtagacagtactaaattcgatcgatacgatttctttaacttctacaatctagatcaactcgataccatcattacagataaccagattagccctcaacacttagaggaatttagccagtacactactattttaaaagcggactag

SEQ ID NO: 98 - protein, 255 aa, Lactococcus lactis
MNKKRRLEKILDMLKIDGTITIKEIIDELDISDMTARRDLDALEADGLLTRTHGGAQLLSSKKPLEKTHIEKKSLNTKEKIDIAKKACSLIKDGDTIFIGPGTTLVQLALELKGRKGYKIRVITNSLPVFLILNDSETIDLLLLGGEYREITGAFVGSMASTNLKAMRFAKAFVSANAVTHNSIATYSDKEGVIQQLALNNAVEKFLLVDSTKFDRYDFFNFYNLDQLDTIITDNQISPQHLEEFSQYTTILKAD

SEQ ID NO: 99 - DNA, 20 bp, Lactococcus lactis
  primer_bind (1)..(20): 43 (T3) primer
aattaaccctcactaaaggg

SEQ ID NO: 100 - DNA, 18 bp, Escherichia coli
  primer_bind (1)..(18): Primer reverse for 5′ end of gusA.
cgcgatccagactgaatg

SEQ ID NO: 101 - DNA, 22 bp, Lactococcus lactis
  primer_bind (1)..(22): Primer 603
atcaacctttgatacaaggttg

SEQ ID NO: 102 - DNA, 120 bp, Lactococcus lactis
  promoter (1)..(120): Synthetic promoter to drive pdc expression where N=any of A, T, G or C
  misc_feature (24)..(28): n is a, c, g, or t
  misc_feature (44)..(57): n is a, c, g, or t
  misc_feature (67)..(70): n is a, c, g, or t
ctagtctagactgaaccctaccgnnnnnagtttattcttgacannnnnnnnnnnnnntgrtataatnnnnaagtaataaaatattcggaggaattttgaaatgagttatacagtaggaac

SEQ ID NO: 103 - DNA, 37 bp, Lactococcus lactis
  primer_bind (1)..(37): Primer used with 690, PstI
tcggactgcagttacaacaatttatttacaggttttc

SEQ ID NO: 104 - DNA, 19 bp, Lactococcus lactis
  primer_bind (1)..(19): Primer 702; pLB85
gagtcgacctgcagaattc

SEQ ID NO: 105 - DNA, 19 bp, Lactococcus lactis
  primer_bind (1)..(19): Primer 703; pLB85, XbaI
tcgactctagaggatctac

SEQ ID NO: 106 - DNA, 37 bp, Lactococcus lactis
  primer_bind (1)..(37): Primer 768
aattcctgcagcatattaaataatgaacaagtcattc

SEQ ID NO: 107 - DNA, 32 bp, Lactococcus lactis
  primer_bind (1)..(32): Primer 769
tagtggatcctggtaaatccaaacacaacaac

SEQ ID NO: 108 - DNA, 34 bp, Lactococcus lactis
  primer_bind (1)..(34): Primer 770
aattcctgcagtaatttccagctcttacaataac

SEQ ID NO: 109 - DNA, 31 bp, Lactococcus lactis
  primer_bind (1)..(31): Primer 771
gacctcgagtcagaaactttctttaccagag

SEQ ID NO: 110 - DNA, 21 bp, Lactococcus lactis
  primer_bind (1)..(21): Primer 772
gcggggatccactagttctag

SEQ ID NO: 111 - DNA, 17 bp, Lactococcus lactis
  primer_bind (1)..(17): Primer 773
ataccgtcgacctcgag

SEQ ID NO: 112 - DNA, 30 bp, Lactococcus lactis
  primer_bind (1)..(30): Primer 774
tagtggatccctgtttcaggtcttggatag

SEQ ID NO: 113 - DNA, 33 bp, Lactococcus lactis
  primer_bind (1)..(33): Primer 775
ccgatgaattctcattagcacgtttaacaagag

SEQ ID NO: 114 - DNA, 29 bp, Lactococcus lactis
  primer_bind (1)..(29): Primer 776
ccgatgaattcatcagcgtagtctgctgc

SEQ ID NO: 115 - DNA, 33 bp, Lactococcus lactis
  primer_bind (1)..(33): Primer 777
cggggtaccatttaatcctaaagtcgttattac

SEQ ID NO: 116 - DNA, 32 bp, Lactococcus lactis
  primer_bind (1)..(32): Primer 785
ccgatgaattcttaagtcaagacaacgaggtc

SEQ ID NO: 117 - DNA, 33 bp, Lactococcus lactis
  primer_bind (1)..(33): Primer 786
ccgatgaattcgaccttgttgaaaaaaatcttc

SEQ ID NO: 118 - DNA, 30 bp, Lactococcus lactis
  primer_bind (1)..(30): Primer 787
tagtggatccgtacaatggctactgttaac

SEQ ID NO: 119 - DNA, 33 bp, Lactococcus lactis
  primer_bind (1)..(33): Primer 788
gacctcgaggatgaacagacttttttattatag

SEQ ID NO: 120 - DNA, 20 bp, Lactococcus lactis
  primer_bind (1)..(20): Primer 789
aaaaccaggtgaaactcgtc

SEQ ID NO: 121 - DNA, 38 bp, Lactococcus lactis
  primer_bind (1)..(38): Primer 791
tcggactgcagttaaaatgctgataaaaacaattcttc

SEQ ID NO: 122 - DNA, 17 bp, Lactococcus lactis
  primer_bind (1)..(17): Primer 827
ataccgtcgacctcgag

SEQ ID NO: 123 - DNA, 22 bp, Lactococcus lactis
  primer_bind (1)..(22): Primer 828
cgataagcttgatatcgaattc

SEQ ID NO: 124 - DNA, 37 bp, Lactococcus lactis
  primer_bind (1)..(37): Primer 829 used with 690, EcoRI
ccgatgaattcttacaacaatttatttacaggttttc

SEQ ID NO: 125 - DNA, 52 bp, Lactococcus lactis
  primer_bind (1)..(52): Primer 830
ccgatgaattctataaggagaattagaatggcaagtagtacattttatattc

SEQ ID NO: 126 - DNA, 30 bp, Lactococcus lactis
  primer_bind (1)..(30): Primer 834 for pfl ups., XbaI
ctagtctagacaagtgatgtaccaaatgac

SEQ ID NO: 127 - DNA, 29 bp, Lactococcus lactis
  primer_bind (1)..(29): Primer 835 for pfl ups., BamHI
cgcggatcctttgaaatctcctttgttct

SEQ ID NO: 128 - DNA, 33 bp, Lactococcus lactis
  primer_bind (1)..(33): Primer 836 for pfl dwn., BamHI
cgcggatccttcttagtattaaaaaatataaag

SEQ ID NO: 129 - DNA, 30 bp, Lactococcus lactis
  primer_bind (1)..(30): Primer 837; pfl dwn., XhoI
ggtactcgagtgtgattcacccctatttct

SEQ ID NO: 130 - DNA, 98 bp, Lactococcus lactis
  promoter (1)..(98): Synthetic promoter primer 845, pdc, XbaI -
ctagtctagactgaaccctaccgtggggagtttattcttgacaagttctcttggagttgatataatcaagaagtaataaaatattcggaggaattttg

SEQ ID NO: 131 - DNA, 98 bp, Lactococcus lactis
  promoter (1)..(98): Synthetic promoter primer 846, pdc, XbaI
ctagtctagactgaaccctaccggtcagagtttattcttgacatcgttaccgtaggttggtataatcttgaagtaataaaatattcggaggaattttg

SEQ ID NO: 132 - DNA, 98 bp, Lactococcus lactis
  promoter (1)..(98): Synthetic promoter primer 847, pdc, XbaI
ctagtctagactgaaccctaccggcaagagtttattcttgacaattacctgttcgcttgatataataaggaagtaataaaatattcggaggaattttg

SEQ ID NO: 133 - DNA, 98 bp, Lactococcus lactis
  promoter (1)..(98): Synthetic promoter primer 848, pdc, XbaI
ctagtctagactgaaccctaccggtcgaagtttattcttgacaatctgtgagatcagtgatataatacagaagtaataaaatattcggaggaattttg

SEQ ID NO: 134 - DNA, 21 bp, Lactococcus lactis
  primer_bind (1)..(21): Primer 878
atccctcggttacaagtttcu

SEQ ID NO: 135 - DNA, 45 bp, Lactococcus lactis
  primer_bind (1)..(45): Primer 879
agaaacttgtaaccgagggauaataatagattgaaattctgtcag

SEQ ID NO: 136 - DNA, 46 bp, Lactococcus lactis
  primer_bind (1)..(46): Primer 880
attcgatatcaagcttatcgaucaaaaattgtggtagaatatatag

SEQ ID NO: 137 - DNA, 40 bp, Lactococcus lactis
  primer_bind (1)..(40): Primer 881
aggtcgacggtatcgataaucctagttcaattgatgtgac

SEQ ID NO: 138 - DNA, 22 bp, Lactococcus lactis
  primer_bind (1)..(22): Primer 882
atcgataagcttgatatcgaau

SEQ ID NO: 139 - DNA, 20 bp, Lactococcus lactis
  primer_bind (1)..(20): Primer 883
attatcgataccgtcgaccu

SEQ ID NO: 140 - DNA, 23 bp, Lactococcus lactis
  primer_bind (1)..(23): Primer 891
acagattaaaggttgaccagtau

SEQ ID NO: 141 - DNA, 20 bp, Lactococcus lactis
  primer_bind (1)..(20): Primer 892
accaattctgtgttgcgcau

SEQ ID NO: 142 - DNA, 49 bp, Lactococcus lactis
  primer_bind (1)..(49): Primer 894
atactggtcaacctttaatctguttaaaatgctgataaaaacaattctt

SEQ ID NO: 143 - DNA, 22 bp, Lactococcus lactis
  primer_bind (1)..(22): Primer 920
ataagctugatatcgaattcct

SEQ ID NO: 144 - DNA, 22 bp, Lactococcus lactis
  primer_bind (1)..(22): Primer 921
attcccttuagtgagggttaat

SEQ ID NO: 145 - DNA, 21 bp, Lactococcus lactis
  primer_bind (1)..(21): Primer 927
atgtgtacgutctcctttgtg

SEQ ID NO: 146 - DNA, 32 bp, Lactococcus lactis
  primer_bind (1)..(32): Primer 928
acgtacacauattatagtatttggaaccgaac

SEQ ID NO: 147 - DNA, 27 bp, Lactococcus lactis
  primer_bind (1)..(27): Primer 929
aagcttauggtcgtcttgttacttgtg

SEQ ID NO: 148 - DNA, 28 bp, Lactococcus lactis
  primer_bind (1)..(28): Primer 930
aaagggaautctgccggagctatatatg

SEQ ID NO: 149 - DNA, 95 bp, Lactococcus lactis
  promoter (1)..(95): Promoter primer 947 to modulate pdc expression, USER
  misc_feature (25)..(29): n is a, c, g, t or u
  misc_feature (45)..(58): n is a, c, g, t or u
  misc_feature (68)..(71): n is a, c, g, t or u
atgcgcaacacagaattgguggccnnnnnagtttattcyyramannnnnnnnnnnnnntgryayaaynnnnaagtaataaaatattcggaggaat

SEQ ID NO: 150 - DNA, 93 bp, Lactococcus lactis
  primer_bind (1)..(93): Primer ptd1 - gusA fwd
gactgaactagtagatctgcagaagcttgtcgacccgggtacctcgagctccatggcatatgcggccgcatgcgcaacacagaattggttaac

SEQ ID NO: 151 - DNA, 84 bp, Lactococcus lactis
  primer_bind (1)..(84): Primer ptd2 gusA rev.
acctctcctaggattaaaaaataaaaaagaacccactcgggttcttttttttactagctagctaatggtgcgccaggagagttg

SEQ ID NO: 152 - DNA, 43 bp, Lactococcus lactis
  primer_bind (1)..(43): Primer ptd45 gusA rev.
ttctgcagatcttcagtcagtcaagaggtttgatgactttgac

SEQ ID NO: 153 - DNA, 32 bp, Lactococcus lactis
  primer_bind (1)..(32): Primer ptd46; pAK80
tttaatcctagggattgtgggaaatttaggcg

SEQ ID NO: 154 - DNA, 32 bp, Lactococcus lactis
  primer_bind (1)..(32): Primer ptd49; pTD5, StuI
atgctagggcccacggggaatttgtatcgatg

SEQ ID NO: 155 - DNA, 32 bp, Lactococcus lactis
  primer_bind (1)..(32): Primer ptd50; pTD5, ApaI
tgataaaggccttactgcactatcaacacact

SEQ ID NO: 156 - DNA, 30 bp, Lactococcus lactis
  primer_bind (1)..(30): Primer ptd51; pG+host8, StuI
ttaagaaggcctaagaaatttcgccagtcg

SEQ ID NO: 157 - DNA, 31 bp, Lactococcus lactis
  primer_bind (1)..(31): Primer Ptd52;  pG+host8, ApaI
gcgcctgggcccgccactcatagttctaaac

(19) United States
(12) Patent Application Publication
(10) Pub. No.: US 2019/0048368 A1
(43) Pub. Date: Feb. 14, 2019

(54) A BACTERIAL CELL FACTORY FOR EFFICIENT PRODUCTION OF ETHANOL FROM WHEY

(71) Applicant: Danmarks Tekniske Universitet, Kgs. Lyngby (DK)

(72) Inventors: Peter Ruhdal Jensen, Gentofte (DK); Jianming Liu, Virum (DK); Christian Solem, Nærum (DK); Shruti Harnal Dantoft, Værløse (DK)

(21) Appl. No.: 16/079,683

(22) PCT Filed: Feb. 24, 2017
(86) PCT No.: PCT/EP2017/054347

§ 371 Date: Aug. 24, 2018

(30) Foreign Application Priority Data
Feb. 25, 2016 (EP) 16157325.8

(51) Int. Cl. C12P 7/06; C12N 15/75; C12R 1/225; C12R 1/46; C12R 1/07; C12N 9/88; C12N 9/04; C12N 9/02; C12N 9/12; C12N 9/24; C12N 9/90

(52) U.S. Cl. CPC C12P 7/06; C12Y 503/01026; C12R 1/225; C12R 1/46; C12R 1/07; C12N 9/88; C12N 9/0006; C12N 9/0008; C12N 9/1205; C12N 9/1223; C12N 9/2402; C12N 9/90; C12Y 401/01001; C12Y 101/01001; C12Y 101/01027; C12Y 101/01028; C12Y 102/0101; C12Y 207/01069; C12Y 207/01114; C12Y 203/01008; C12Y 302/01085; C12N 15/75

(57) ABSTRACT

The invention relates to a method for homo-ethanol production from lactose using a genetically modified lactic acid bacterium of the invention, where the cells are provided with a substrate comprising dairy waste supplemented with an amino nitrogen source (such as acid hydrolysed corn steep liquor). The invention further relates to genetically modified lactic acid bacterium and its use for homo-ethanol production from lactose in dairy waste. The lactic acid bacterium comprises both genes (lacABCD, LacEF, lacG) encoding enzymes catalysing the lactose catabolism pathway; and transgenes (pdc and adhB) encoding enzymes catalysing the conversion of pyruvate to ethanol. Additionally a number of genes (ldh, pta and adhE) are deleted in order to maximise homo-ethanol production as compared to production of lactate, acetoin and acetate production.

### FIELD OF THE INVENTION

The invention relates to a method for homo-ethanol production from lactose using a genetically modified lactic acid bacterium, where cells of the bacterium are provided with a substrate comprising dairy waste supplemented with an amino nitrogen source, such as hydrolysed corn steep liquor as a source of soluble protein, peptides and free amino acids. Additionally the invention provides the genetically modified lactic acid bacterium that is adapted for homo-ethanol production from lactose in dairy waste, such as whey, deproteinized whey permeate or permeate mother liquor, which is the byproduct after lactose extraction from whey permeate, and for its use for homo-ethanol production. The genetically modified lactic acid bacterium comprises both genes (lacABCD, lacEF, lacG) encoding enzymes catalysing a lactose catabolism pathway; and transgenes (pdc and adhB) encoding enzymes catalysing the conversion of pyruvate to ethanol. The lactic acid bacterium is further genetically modified by inactivation or deletion of those genes in its genome that encode polypeptides having lactate dehydrogenase (EC 1.1.1.27/EC 1.1.1.28); phosphotransacetylase (EC 2.3.1.8), and bifunctional alcohol dehydrogenase (EC 1.1.1.1 and EC 1.2.1.10) activity.

### BACKGROUND OF THE INVENTION

Currently there is a growing demand for liquid fuels that can be produced in a sustainable manner from renewable raw materials. The potential that lies in using microorganisms for converting various feedstocks, e.g. plant biomass, into useful compounds including fuels, has already been recognized, and intense research is being carried out to establish robust and economically feasible processes for production of biofuels. Microbially produced ethanol presently dominates the biofuel market, and it is mainly produced from either refined sugar or starch derived sugar. Although much focus has been on developing bio-processes, which are reliant on non-food plant biomass as feedstock, there are many challenges, including the high cost of enzymes needed for degrading the biomass, the recalcitrance of lignocellulose, a lack of microbial catalysts with sufficient robustness to withstand the inhibitors generated in pre-treatment or that have a sufficiently broad spectrum of carbohydrate utilization. As an alternative, one cheap abundant feedstock is cheese whey and its various processed forms, such as whey permeate or whey powder. Whey represents about 85-95% of the milk volume and retains 55% of milk nutrients; and is a liquid byproduct of cheese production, obtained when draining the cheese curd. The worldwide production of cheese whey in 2012 was reported to be 4×10⁷ tons and about 50% hereof was used for animal feed or otherwise disposed of as waste. The latter is a serious problem as whey is discarded as liquid waste and has a high BOD (biochemical oxygen demand) and COD (chemical oxygen demand). The composition of whey varies according to the source of the milk and the technology used for its production. Normally it contains approximately 90% water, 4% lactose, 1% protein, 0.7% minerals and small amounts of vitamins. Separation of whey proteins generates whey-permeate and further extraction of lactose leads to permeate mother liquor (residual whey permeate, RWP), which comprises about 60% lactose on a dry weight basis, as a leftover product (Ling, 2008). Fermentation of the main carbon source in whey (lactose) to ethanol has been studied for the last 30 years and most of the research has been focused on yeasts that naturally metabolize lactose. There are, however, problems associated with yeasts, and these include a generally low robustness, slow fermentation-rate, and substrate-inhibition effects, which is why there is a need for better performing microbial candidates.

Lactococcus lactis, which is well-known for its role in cheese production, has great potential as a cell factory, due to properties such as its high glycolytic flux, ability to metabolize a broad range of carbohydrates, well-characterized metabolic network and ease of genetic manipulation. Its long record of safe use is also an important asset, especially for production of food ingredients. Normally, most of the carbon flux in L. lactis is directed to lactate (homolactic fermentation). However, it can be successfully engineered to produce ethanol by knocking out alternative product pathways and introducing pyruvate decarboxylase and alcohol dehydrogenase heterologously (Solem et al 2013). A potential drawback of using L. lactis as a cell factory is its fastidious nature, i.e. its many nutritional growth requirements, which could perhaps make it less attractive for some industrial applications, e.g. for production of low-priced chemicals where, for competitive reasons, it is important to keep costs at a minimum. Accordingly, there exists a need to develop cheap fermentation media based on nutrient-rich waste substrates that can circumvent problems associated with using L. lactis as a production host organism for ethanol.

### SUMMARY OF THE INVENTION

According to a first embodiment, the invention provides a method for the production of ethanol, comprising the steps of:
a. introducing a genetically modified lactic acid bacterium into an aqueous culture medium;
b. incubating the culture of (a);
c. recovering ethanol produced by said culture in step (b), and optionally
d. isolating the recovered ethanol;
   wherein the aqueous culture medium comprises:
   I. a lactose rich substrate, such as whey, whey permeate or residual whey permeate, and
   II. an amino nitrogen source, such as acid hydrolysed corn steep liquor (CSLH) wherein the concentration of at least one free amino acid, selected from the group consisting on glutamine, histidine, methionine, leucine, isoleucine, and valine in the CSLH is at least 1.5 fold greater than the concentration of the corresponding amino acid in the original corn steep liquor (CAS Number: 66071-94-1) from which the CSLH was derived, and
   wherein the genetically engineered lactic acid bacterium comprises transgenes encoding:
   i. a polypeptide having pyruvate decarboxylase (PDC) activity (EC 4.1.1.1); and
   ii. a polypeptide having alcohol dehydrogenase B activity (EC 1.1.1.1); and
      wherein the genome of said lactic acid bacterium comprises genes encoding polypeptides having:
   iv. lactose-specific phosphotransferase system (PTS) activity (EC 2.7.1.69)
   v. phospho-β-D-galactosidase activity (EC 3.2.1.85)
   vi. galactose-6-phosphate isomerase activity (EC 5.3.1.26),
   vii. D-tagatose-6-phosphate kinase activity (EC 2.7.1.114), and
   viii. tagatose 1,6-diphosphate aldolase activity (EC 4.1.2.40);
      wherein the genome of said lactic acid bacterium is deleted for genes or lacks genes or lacks functional genes encoding polypeptides having an enzymatic activity of:
   ix. lactate dehydrogenase (E.C 1.1.1.27 or E.C. 1.1.1.28)
   x. phosphotransacetylase (E.C. 2.3.1.8) and
   xi. bifunctional alcohol dehydrogenase (E.C. 1.1.1.1 and EC 1.2.1.10).

According to a second embodiment, the invention provides for a use of a genetically engineered lactic acid bacterium for the production of ethanol from an aqueous culture medium comprising a lactose rich substrate (such as whey, whey permeate or residual whey permeate), and an amino nitrogen source (such as acid hydrolysed corn steep liquor (CSLH) as defined herein); wherein the genetically engineered lactic acid bacterium comprises transgenes encoding:
i. a polypeptide having pyruvate decarboxylase (PDC) activity (EC 4.1.1.1); and
ii. a polypeptide having alcohol dehydrogenase B activity (EC 1.1.1.1); and
   wherein the genome of said lactic acid bacterium comprises genes encoding polypeptides having:
iii. lactose-specific phosphotransferase system (PTS) activity (EC 2.7.1.69)
iv. phospho-β-D-galactosidase activity (EC 3.2.1.85)
v. galactose-6-phosphate isomerase activity (EC 5.3.1.26),
vi. D-tagatose-6-phosphate kinase activity (EC 2.7.1.114), and vii. tagatose 1,6-diphosphate aldolase activity (EC 4.1.2.40); and
   wherein the genome of said lactic acid bacterium is deleted for genes or lacks genes or lacks functional genes encoding polypeptides having an enzymatic activity of:
viii. lactate dehydrogenase (E.C 1.1.1.27 or E.C. 1.1.1.28)
ix. phosphotransacetylase (E.C. 2.3.1.8) and
x. bifunctional alcohol dehydrogenase (E.C. 1.1.1.1 and EC 1.2.1.10).

According to a third embodiment, the invention provides the genetically modified lactic acid bacterium for production of ethanol for the production of ethanol from an aqueous culture medium comprising residual a lactose rich substrate (such as whey, whey permeate or whey permeate), and an amino nitrogen source (such as acid hydrolysed corn steep liquor (CSLH)); where the bacterium is as defined above.

### DESCRIPTION OF THE INVENTION

### Description of the Figures

FIG. 1. Cartoon showing sugar metabolism pathway in Lactococcus lactis. The pathway includes lacEF genes encoding lactose-specific phosphotransferase system; lacG gene encoding phospho-β-galactosidase; lacAB genes encoding galactose-6-phosphate isomerase; LacC gene encoding D-tagatose-6-phosphate kinase, lacD gene encoding tagatose 1,6-diphosphate aldolase, Abbreviations: CSLH, corn steep liquor hydrolysate; AA, amino acids; G3P, glyceraldehyde 3-phosphate; LDH: lactate dehydrogenase, PTA: phosphotransacetylase; ADHE: native alcohol dehydrogenase. PDC: pyruvate decarboxylase and ADHB, alcohol dehydrogenase, are both encoded by codon-optimized synthetic genes based on the Zymomonas mobilis sequences. Crosses indicate knock-out of enzyme functions in the genetically modified L. lactis strains.

FIG. 2. Cartoon showing the genes of the lactose catabolism operon, that is present on the pLP712 plasmid derived from industrial dairy starter strain NCDO712. The plasmid confers on a cell the ability to take up lactose via a lactose-specific phosphotransferase system (PTS), encoded by lacEF genes, where after phosphorylated lactose is hydrolyzed to glucose and galactose-6-phosphate (gal-6-P) by the phospho-β-galactosidase (lacG gene). The glucose moiety enters into glycolysis, while gal-6-P is degraded via the tagatose-6-P pathway (lacABCD genes).

FIG. 3. Graph showing the performance of L. lactis strain CS4435L when grown in defined SA medium supplemented with 7.2 g/L lactose (instead of glucose) as the only energy source. The graphs displays: cell density, measured as OD600ₙₘ (filled squares); lactose concentration as g/L (filled circles) and ethanol concentration as g/L (filled triangles). The experiments were conducted in duplicate and error bars indicate standard deviations.

FIG. 4. Graph showing growth and ethanol production of L. lactis strain CS4435L when cultured on residual whey permeate (RWP) alone, or RWP supplemented with the indicated amounts (calculated on a weight per volume basis) of YE, yeast extract; CSL, corn steep liquor; CSLH, corn steep liquor hydrolysate; H1-H3, different hydrolysis conditions (H1: CSL treated with 0.05% H₂SO₄; H2: CSL treated with 0.25% H₂SO₄; H3: CSL treated with 0.5% H₂SO₄). The whey medium comprised three-fold diluted RWP and 50 g/L lactose. After 30 hours in culture, cell growth was determined as final cell density (OD600ₙₘ); and samples from each culture were collected for measurement of ethanol content. Experiments were conducted at least in duplicate and error bars indicate standard deviations.

FIG. 5. Graph showing growth and ethanol production of L. lactis strain CS4435L on a medium of only diluted residual whey permeate medium (without the addition of any vitamin or minerals) supplemented with yeast extract (YE) or corn steep liquor hydrolysate (CSLH) as amino-nitrogen sources. (A) The fermentation was carried out in diluted RWP medium containing initially 32 g/L lactose and 0.5% (w/v) yeast extract; (B) The fermentation was carried out in diluted RWP medium containing initially 40 g/L lactose and 2.5% (w/v) CSLH. CSLH was prepared based on H1 conditions (CSL treated with 0.05% H₂SO₄). Samples were collected periodically for determining cell density measured at OD600ₙₘ (filled squares), lactose concentration (filled circles) and ethanol concentration (filled triangles) are displayed. Experiments were conducted in duplicate and error bars indicate standard deviations.

FIG. 6. Graph showing growth and ethanol production of the L. lactis strain CS4435L on diluted residual whey permeate medium containing initially 80 g/L lactose and 2.5% (w/v) CSLH over 55 hours. CSLH was prepared based on H1 conditions (CSL treated with 0.05% H₂SO₄). Samples were collected periodically for determining cell density measured at OD600ₙₘ (filled squares), lactose concentration (filled circles) and ethanol concentration (filled triangles), as displayed. Experiments were conducted in duplicate and error bars indicate standard deviations.

FIG. 7. Graph showing growth and ethanol production of the L. lactis strain CS4435L during fed-batch culturing. Fed-batch was performed with an initial medium comprising 80 g/L lactose and 2.5% (w/v) CSLH in the residual whey permeate medium, and 500 g/L lactose stock solution was used for feeding. CSLH was prepared based on H1 conditions (CSL treated with 0.05% H₂SO₄). Samples were collected periodically for determining cell density (filled squares), lactose concentration (filled circles) and ethanol concentration (filled triangles) as displayed. Experiments were conducted in duplicate and error bars indicate standard deviations.

FIG. 8. Graph showing ethanol production by the L. lactis strain CS4435L when cultured in 3 different growth media, each comprising diluted residual whey permeate medium and 80 g/L lactose and supplemented with one of: (1) 2.5% (w/v) CSLH prepared under H1 conditions (CSL treated with 0.05% H₂SO₄); (2) 2.5% (w/v) CSL and 70 IU/L proteinase; and (3) 2.5% (w/v) CSL and 700 IU/L proteinase (Aspergillus melleus). Samples were collected periodically for determining ethanol concentration (filled triangles), over a period of 40-50 hours as displayed.

### ABBREVIATIONS AND TERMS

Amino acid sequence identity: The term “sequence identity” as used herein, indicates a quantitative measure of the degree of homology between two amino acid sequences of substantially equal length. The two sequences to be compared must be aligned to give a best possible fit, by means of the insertion of gaps or alternatively, truncation at the ends of the protein sequences. The sequence identity can be calculated as ((Nref−Ndif)100)/(Nref), wherein Ndif is the total number of non-identical residues in the two sequences when aligned and wherein Nref is the number of residues in one of the sequences. This sequence identity obtained using the BLAST program e.g. the BLASTP program (Pearson W. R and D. J. Lipman (1988)) (www.ncbi.nlm.nih.gov/cgi-bin/BLAST). Sequence alignment is performed with the sequence alignment method ClustalW with default parameters as described by Thompson J., et al 1994, available at http://www2.ebi.ac.uk/clustalw/.

Preferably, the numbers of substitutions, insertions, additions or deletions of one or more amino acid residues in the polypeptide as compared to its comparator polypeptide is limited, i.e. no more than 1, 2, 3, 4, 5, 6, 7, 8, 9, or 10 substitutions, no more than 1, 2, 3, 4, 5, 6, 7, 8, 9, or 10 insertions, no more than 1, 2, 3, 4, 5, 6, 7, 8, 9, or 10 additions, and no more than 1, 2, 3, 4, 5, 6, 7, 8, 9, or 10 deletions. Preferably the substitutions are conservative amino acid substitutions: limited to exchanges within members of group 1: Glycine, Alanine, Valine, Leucine, Isoleucine; group 2: Serine, Cysteine, Selenocysteine, Threonine, Methionine; group 3: Proline; group 4: Phenylalanine, Tyrosine, Tryptophan; Group 5: Aspartate, Glutamate, Asparagine, Glutamine.

Corn steep liquor: has CAS Number: 66071-94-1 and is a by-product of corn wet-milling industry. It contains proteins, amino acids, vitamins and minerals and contains approx. 50% (w/w) solids.

Deleted gene: the deletion of a gene from the genome of a bacterial cell leads to a loss of function of the gene and hence where the gene encodes a polypeptide the deletion results in a loss of expression of the encoded polypeptide. Where the encoded polypeptide is an enzyme, the gene deletion leads to a loss of detectable enzymatic activity of the respect polypeptide in the bacterial cell.

Functional gene: gene that is capable of expressing an active enzyme encoded by the gene. Loss of a gene or loss of the function of a gene results in an inability to express the active enzyme encoded by the gene. A loss of function may be the result of a failure to transcribe the gene; or may be a failure to translate the transcribed gene into an active enzyme (e.g. due to mutations). When an enzyme loses more than 60% activity, preferably more than 90% activity; it is deemed to be inactive, in the sense that it no longer has a significant influence on product flux in the sugar metabolism pathway.

gi number: (genInfo identifier) is a unique integer which identifies a particular sequence, independent of the database source, which is assigned by NCBI to all sequences processed into Entrez, including nucleotide sequences from DDBJ/EMBL/GenBank, protein sequences from SWISS-PROT, PIR and many others.

Native gene: endogenous gene in a bacterial cell genome (where the genome includes plasmids), where the gene is homologous to the host bacterium.

Transgenes encoding polypeptides having pyruvate decarboxylase (PDC) activity (EC 4.1.1.1) and alcohol dehydrogenase (ADHB) activity (EC 1.1.1.1) confer on a cell the ability to convert pyruvate to ethanol via acetaldehyde.

Whey and whey permeate and residual whey permeate: whey is a byproduct of cheese manufacture; and comprises whey proteins having a high nutritional value and lactose. Removal of whey proteins, typically by means of ultrafiltration or diafiltration produces a whey protein concentrate and whey permeate that is lactose-rich. The lactose content of the whey permeate is dependent on the treatment conditions and typically it can reach as high as hundreds of grams per liter by reverse osmosis, such as 200 g/L. Removal of fat from whey, or from lactose-rich permeate, typically by centrifugation, yields a fat-free composition (whey or permeate). Residual whey permeate (also called permeate mother liquor) is obtained after the extraction of lactose from whey permeate (typically by lactose crystallisation); and has a lower lactose content of about 150 g/L.

### DETAILED DESCRIPTION OF THE INVENTION

### I: A Genetically Modified Lactic Acid Bacterium for the Production of Ethanol from Lactose

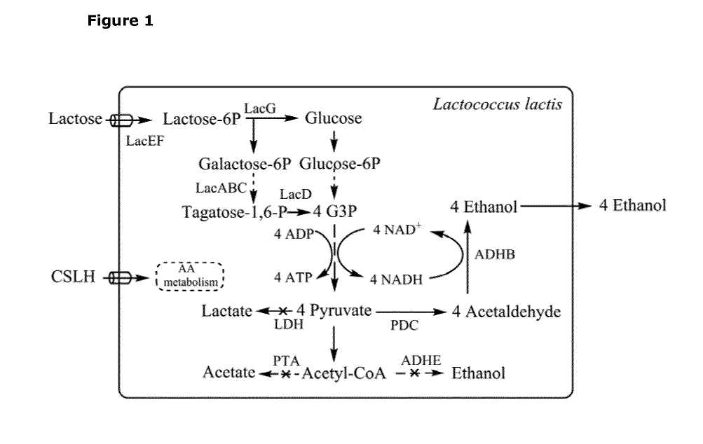

Lactococcus lactis is a homo-lactic fermentative lactic acid bacterium, directing about 90% of metabolic flux to lactate when grown on fast fermentable sugars, such as glucose (FIG. 1). The wild-type L. lactis strain MG1363 exemplifies the homolactic fermentation of L. lactis when grown on glucose, as shown in Table 3 of Example 1. L. lactis is naturally capable of producing ethanol, but only in small amounts. Thus a number of genetic modifications are required to redirect L. lactis metabolism towards homo-ethanol production, as described below.

In order to limit metabolic flux towards lactate in the lactic acid bacterium of the invention, the activity of the enzymes of the lactate pathway are reduced by inactivating or deleting one or more genes, for example ldh, ldhX and ldhB, encoding enzymes of this pathway. In order to additionally prevent metabolic flux to acetate, the activity of the acetate pathway is reduced by inactivating or deleting the gene encoding phosphotransacetylase (pta), which converts acetylphosphate to acetate. Additionally, the native alcohol dehydrogenase gene (adhE) encoding a bifunctional alcohol dehydrogenase, ADHE (EC 1.1.1.1 and EC 1.2.1.10) may be inactivated or deleted in order to maximize the production of ethanol with a high yield. So long as the native alcohol dehydrogenase (ADHE) is active, another byproduct (acetoin) is formed in order to balance the cofactors (2 NADH is formed per glucose by glycolysis, while 4 NADH is required for the complete reduction to ethanol by ADHE, which means only half of the carbon flux can be diverted to ethanol). Therefore, the removal of ADHE activity is beneficial for high yield ethanol production.

The lactic acid bacterium of the invention, further comprises codon-optimized transgenes (pdc and adhB), sourced from Zymomonas moblis, encoding pyruvate decarboxylase (EC 4.1.1.1) and alcohol dehydrogenase (EC 1.1.1.1) enzymes, respectively. Expression of these encoded PDC and ADHB enzymes in cells of the lactic acid bacterium of the invention, where the lactate and acetate pathways are inactivated or deleted, confers the ability for homo-ethanol production and for growth under anaerobic growth conditions (see Strain CS4435 in Table 3 of Example 1). The above described genetic modifications in the lactic acid bacterium of the invention provide the cells with a metabolic pathway for the use of (extracellular) glucose as substrate for homo-ethanol production.

The lactic acid bacterium of the invention, however, further comprises genes encoding enzymes that facilitate the uptake of extracellular lactose and its entry into the glycolytic pathway; such that lactate can be used as substrate for homo-ethanol production. Preferably, the cells of the lactic acid bacterium comprise the following genes: a gene encoding a lactose specific phosphotransferase system (PTS) e.g., a lacEF gene, whereby phosphorylated lactose is assimilated by the cells; a gene encoding a phospho-β-galactosidase (e.g. a lacG gene) that hydrolyzes lactose phosphate to glucose and galactose-6-phosphate (gal-6-P), where the glucose moiety enters into glycolysis; genes encoding the tagatose-6-P pathway, e.g., the lacAB, lacC, lacD genes encoding galactose-6-phosphate isomerase, D-tagatose-6-phosphate kinase, and tagatose 1,6-diphosphate aldolase, respectively, whereby gal-6-P is degraded and enters the glycolytic pathway as glyceraldehyde-3-phosphate.

Cells of the lactic acid bacterium of the invention are shown to be efficient producers of ethanol from lactose via homo-ethanol fermentation with a growth rate that is close to that on a glucose-containing medium (Example 1).

The characteristics of the individual genes that may be deleted or introduced in order to produce a lactic acid bacterium of the invention are detailed below.

### I.i Deletion of the Lactate Pathway:

The lactic acid bacterium of the invention is characterised by knockouts of one or more endogenous native gene encoding a polypeptide having lactate dehydrogenase activity causing a block in the lactate synthesis pathway in the bacterium. Deletion of at least one gene (e.g. ldh) encoding a lactate dehydrogenase enzyme (E.C 1.1.1.27 or EC 1.1.1.28) provides a lactic acid bacterium of the invention that is depleted in lactate production. For example, where the lactic acid bacterium of the invention belongs to a given genus, the deleted endogenous gene is one encoding a polypeptide having lactate dehydrogenase activity in that genus. Preferably the polypeptide having lactate dehydrogenase activity (EC 1.1.1.27 or EC 1.1.1.28) has at least 70, 72, 74, 76, 78, 80, 82, 84, 86, 88, 90, 92, 94, 96, 98, 99 or 100% amino acid sequence identity to one of the following sequences: SEQ ID NO: 2 in a Lactococcus species (e.g. Lactococcus lactis); SEQ ID NO: 4, 6, or 8 in a Lactobacillus species (e.g. Lactobacillus acidophilus); SEQ ID NO: 10 in a Lactobacillus species (e.g. Lactobacillus delbrueckii); SEQ ID NO. 12, 14 or 16 in a Lactobacillus species (e.g. Lactobacillus casei), SEQ ID NO. 18 or 20 in a Lactobacillus species (e.g. Lactobacillus plantarum); SEQ ID NO: 22 in a Pediococcus species (e.g. Pediococcus pentosaceus), SEQ ID NO: 24 or 26 in a Leuconostoc species (e.g. Leuconostoc mesenteroides), SEQ ID NO: 28 in a Streptococcus species (e.g. Streptococcus thermophilus), SEQ ID NO: 30 or 32 in a Oenococcus species (e.g. Oenococcus oeni), and SEQ ID NO: 34 or 36 in a Bacillus species (e.g. Bacillus coagulans).

In one embodiment, an additional endogenous gene, encoding a polypeptide having lactate dehydrogenase enzymatic activity (E.C 1.1.1.27 or EC1.1.1.28), is deleted from the lactic acid bacterium of the invention. For example, where the lactic acid bacterium of the invention belongs to the genus Lactococcus, the deleted gene (ldhX) encodes a polypeptide having at least 70, 72, 74, 76, 78, 80, 82, 84, 86, 88, 90, 92, 94, 96, 98, 99 or 100% amino acid sequence identity to SEQ ID NO: 38.

In one embodiment, an additional endogenous gene, encoding a polypeptide having lactate dehydrogenase enzymatic activity (EC 1.1.1.27 or EC 1.1.1.28), is deleted from the lactic acid bacterium of the invention. For example, where the lactic acid bacterium of the invention belongs to the genus Lactococcus, the deleted gene (ldhB) encodes a polypeptide having at least 70, 72, 74, 76, 78, 80, 82, 84, 86, 88, 90, 92, 94, 96, 98, 99 or 100% amino acid sequence identity to SEQ ID NO: 40. Further, where the lactic acid bacterium of the invention belongs to the genus Lactococcus, the three genes (ldh, ldhB and ldhX) encoding a polypeptide having at least 70% amino acid sequence identity to SEQ ID NO: 2, 38 and 40 respectively may be deleted.

### I.ii Deletion of the Acetate Pathway:

In one embodiment, the lactic acid bacterium of the invention is characterised by knockout of the endogenous native gene encoding a phosphotransacetylase (EC 2.3.1.8), causing a block in the acetate synthesis pathway in the bacterium. Deletion of a gene (e.g. pta) encoding a phosphotransacetylase enzyme provides a lactic acid bacterium of the invention that is blocked in acetate production. For example, where the lactic acid bacterium of the invention belongs to a given genus, the deleted endogenous gene is one encoding a polypeptide having phosphotransacetylase activity (EC 2.3.1.8) in that genus. Preferably the polypeptide having phosphotransacetylase activity has at least 70, 72, 74, 76, 78, 80, 82, 84, 86, 88, 90, 92, 94, 96, 98, 99 or 100% amino acid sequence identity to one of the following sequences: SEQ ID NO: 42 in a Lactococcus species (e.g. Lactococcus lactis); SEQ ID NO: 44, 46, 48, and 50 in a Lactobacillus species (e.g. Lactobacillus acidophilus, Lactobacillus delbrueckii, Lactobacillus casei, Lactobacillus plantarum), SEQ ID NO: 52 in a Pediococcus species (e.g. Pediococcus pentosaceus), SEQ ID NO: 54 in a Leuconostoc species (e.g. Leuconostoc mesenteroides), SEQ ID NO: 56 in a Streptococcus species (e.g. Streptococcus thermophilus), SEQ ID NO: 58 Oenococcus species (e.g. Oenococcus oeni), and SEQ ID NO: 60 in a Bacillus species (e.g. Bacillus coagulans).

### I.iii Deletion of the Ethanol Pathway:

In one embodiment, the lactic acid bacterium of the invention is characterised by knockout of the endogenous native gene encoding bifunctional alcohol dehydrogenase activity (EC 1.1.1.1 and EC 1.2.1.10) causing a block in the ethanol synthesis pathway in the bacterium. Deletion of the gene encoding an alcohol dehydrogenase enzyme provides a lactic acid bacterium of the invention that is blocked in ethanol production.

For example, where the lactic acid bacterium of the invention belongs to a given genus, the deleted endogenous gene (e.g. adhE) is one encoding a polypeptide having bifunctional alcohol dehydrogenase activity (EC 1.1.1.1 and EC 1.2.1.10) in that genus. Preferably the polypeptide having alcohol dehydrogenase activity has at least 70, 72, 74, 76, 78, 80, 82, 84, 86, 88, 90, 92, 94, 96, 98, 99 or 100% amino acid sequence identity to one of the following sequences: SEQ ID NO: 62 in a Lactococcus species (e.g. Lactococcus lactis); SEQ ID NO: 64 in a Lactobacillus species (e.g. Lactobacillus acidophilus); SEQ ID NO: 66 or 68 in a Lactobacillus species (e.g. Lactobacillus casei); SEQ ID NO: 70 in a Lactobacillus species (e.g., Lactobacillus plantarum), SEQ ID NO: 72 in a Leuconostoc species (e.g. Leuconostoc mesenteroides), SEQ ID NO: 74 in a Streptococcus species (e.g. Streptococcus thermophilus), SEQ ID NO: 76 in a Oenococcus species (e.g. Oenococcus oeni), and SEQ ID NO: 78 in a Bacillus species (e.g. Bacillus coagulans).

### I.iv Transgene Encoding an Enzyme Having Pyruvate Decarboxylase Activity:

The bacterium of the invention comprises a transgene encoding a polypeptide having pyruvate decarboxylase (PDC) activity (EC 4.1.1.1) that converts pyruvate to acetaldehyde. The amino acid sequence of the polypeptide has at least 70, 72, 74, 76, 78, 80, 82, 84, 86, 88, 90, 92, 94, 96, 98, 99 or 100% sequence identity to the amino acid sequence of the pyruvate decarboxylase (SEQ ID NO: 80) encoded by a codon optimized derivative of the Zymomonas mobilis pdc gene.

### I.v Transgene Encoding an Enzyme Having Alcohol Dehydrogenase Activity:

The bacterium of the invention comprises a transgene encoding a polypeptide having alcohol dehydrogenase (ADH) activity (EC 1.1.1.1) that converts acetaldehyde to ethanol, but is not able to use acetyl-CoA as substrate (the acetaldehyde being formed by pyruvate decarboxylase activity). The expression of these heterologous enzymes, PDC and ADHB, enables the complete cofactor balance thereby facilitating maximum ethanol production. The amino acid sequence of the polypeptide has at least 70, 72, 74, 76, 78, 80, 82, 84, 86, 88, 90, 92, 94, 96, 98, 99 or 100% sequence identity to the amino acid sequence of the pyruvate decarboxylase (SEQ ID NO: 82) encoded by a codon optimized derivative of the Zymomonas mobilis adhB gene.

### I.vi Genes Encoding Enzymes of the Lactose Catabolism Pathway:

The lactic acid bacterium of the invention comprises the following native genes or transgenes required for lactose assimilation and catabolism:

1) a first and second gene encoding a first and a second polypeptide component together conferring lactose-specific phosphotransferase system (PTS) activity (EC 2.7.1.69), whereby phosphorylated lactose is assimilated by the cells. The amino acid sequence of the first polypeptide component has at least 70, 72, 74, 76, 78, 80, 82, 84, 86, 88, 90, 92, 94, 96, 98, 99 or 100% sequence identity to the amino acid sequence of the phosphotransferase system EIICB component (SEQ ID NO: 84) encoded by the Lactococcus lactis lacE gene; and the amino acid sequence of the second polypeptide component has at least 70, 72, 74, 76, 78, 80, 82, 84, 86, 88, 90, 92, 94, 96, 98, 99 or 100% sequence identity to the amino acid sequence of the phosphotransferase system EIIA component (SEQ ID NO: 86) encoded by the Lactococcus lactis lacF gene; and
2) a gene encoding a polypeptide having phospho-β-D-galactosidase activity (EC 3.2.1.85) that hydrolyzes lactose-6-phosphate to glucose and galactose-6-phosphate (gal-6-P), whereby the glucose moiety can then enter the glycolytic pathway. The amino acid sequence of the polypeptide has at least 70, 72, 74, 76, 78, 80, 82, 84, 86, 88, 90, 92, 94, 96, 98, 99 or 100% sequence identity to the amino acid sequence of the phospho-β-D-galactosidase (SEQ ID NO: 88) encoded by the Lactococcus lactis lacG gene.

Additionally, the following genes encoding enzymes in the tagatose-6-P pathway, whereby gal-6-P is degraded and enters the glycolytic pathway as glyceraldehyde-3-phosphate, are required:

3) a first and second gene encoding a first and a second polypeptide subunit together conferring galactose-6-phosphate isomerase activity (EC 5.3.1.26). The amino acid sequence of the first polypeptide subunit has at least 70, 72, 74, 76, 78, 80, 82, 84, 86, 88, 90, 92, 94, 96, 98, 99 or 100% sequence identity to the amino acid sequence of the first subunit of the galactose-6-phosphate isomerase (SEQ ID NO: 90) encoded by the Lactococcus lactis lacA gene; and the amino acid sequence of the second polypeptide subunit has at least 70, 72, 74, 76, 78, 80, 82, 84, 86, 88, 90, 92, 94, 96, 98, 99 or 100% sequence identity to the amino acid sequence of the second subunit of the galactose-6-phosphate isomerase (SEQ ID NO: 92) encoded by the Lactococcus lactis lacB gene; and
4) a gene encoding a polypeptide having D-tagatose-6-phosphate kinase activity (EC 2.7.1.114). The amino acid sequence of the polypeptide has at least 70, 72, 74, 76, 78, 80, 82, 84, 86, 88, 90, 92, 94, 96, 98, 99 or 100% sequence identity to the amino acid sequence of the D-tagatose-6-phosphate kinase (SEQ ID NO: 94) encoded by the Lactococcus lactis lacC gene; and
5) a gene encoding a polypeptide having tagatose 1,6-diphosphate aldolase activity (EC 4.1.2.40). The amino acid sequence of the polypeptide has at least 70, 72, 74, 76, 78, 80, 82, 84, 86, 88, 90, 92, 94, 96, 98, 99 or 100% sequence identity to the amino acid sequence of the tagatose 1,6-diphosphate aldolase (SEQ ID NO: 96) encoded by the Lactococcus lactis lacD gene; and
6) optionally a gene encoding a polypeptide having lactose transport regulator activity. The amino acid sequence of the polypeptide has at least 70, 72, 74, 76, 78, 80, 82, 84, 86, 88, 90, 92, 94, 96, 98, 99 or 100% sequence identity to the amino acid sequence of the lactose transport regulator (SEQ ID NO: 98) encoded by the Lactococcus lactis lacR gene.

### I.vii Fermentation Properties of the Lactic Acid Bacterium Genetically Modified for Ethanol Production

Lactococcus lactis is characterised by homo-lactic fermentation when grown on glucose (FIG. 1), as illustrated for the wild-type L. lactis strain MG1363 in Table 3. L. lactis strain CS4435 derived from MG1363 by deletion of the lactate dehydrogenase genes (ldh ldhB, ldhX); the phosphotransacetylase gene (pta); and the alcohol dehydrogenase gene (adhE); and expressing the pyruvate decarboxylase gene (pdc) and ethanol dehydrogenase gene (adhB) derived from Zymomonas mobilis; is characterised by homo-ethanol fermentation when grown on glucose (Table 3).

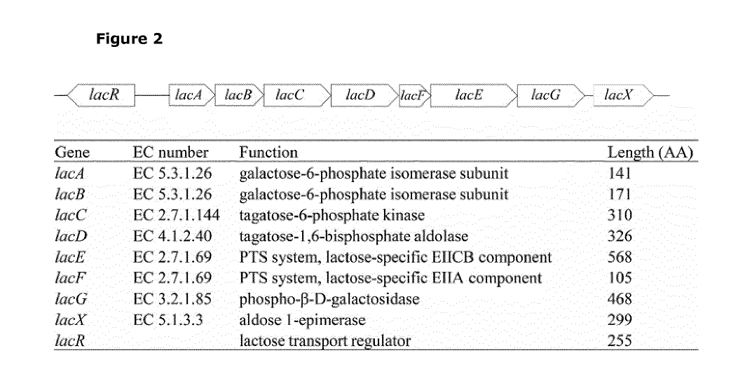

The strain CS4435L, derived from strain CS4435, comprises and expresses the entire lactose catabolism pathway, encoded by genes on the Lactococcal plasmid, pLP712 (55.395 kbp) (FIG. 2). Strain CS4435L is capable of growth on a defined medium with lactose as the sole energy source, at a growth rate of 0.6 h⁻¹, close to that on glucose as sole energy source. The sole detected fermentation product of this strain was ethanol, with an ethanol titer of 3.2 g/L, and a yield of 0.45 g ethanol/g lactose, corresponding to 83% of the theoretical maximum. Higher ethanol titers, of up to 12.0 g/L, were achieved by increasing the initial lactose concentration in the growth medium (Table 2).

### II A Genetically Modified Lactic Acid Bacterium Comprising a Pathway for Homo-Ethanol Production from Lactose in Diary Waste

The lactic acid bacterium according to the invention, that comprises genes encoding a pathway for homo-ethanol production, as described in section I, is a member of a genus of lactic acid bacteria selected from the group consisting of Lactococcus, Lactobacillus, Pediococcus, Leuconostoc, Streptococcus, Oenococcus, and Bacillus, preferably Lactococcus. The lactic acid bacterium of the invention may for example be a species of lactic acid bacteria selected from the group consisting of Lactococcus lactis, Lactobacillus acidophilus, Lactobacillus delbrueckii, Lactobacillus casei, Lactobacillus plantarum, Pediococcus pentosaceus, Leuconostoc mesenteroides, Streptococcus thermophilus, Oenococcus oeni and Bacillus coagulans.

### III a Sustainable and Economical Bioprocess for Ethanol Production Using the Genetically Modified Lactic Acid Bacterium of the Invention

Residual whey permeate (RWP) is the permeate mother liquor after extracting lactose from whey permeate, and is a waste product of the dairy industry. However, its use as a feedstock for ethanol production has the potential to create a sustainable and economical bioprocess for ethanol production. RWP comprises lactose, as a source of energy, as well as the amino acids essential for L. lactis growth, although in relatively low concentrations (Table 5).

A growth medium, based on RWP alone, was found insufficient to support either fermentative growth or ethanol production by the genetically modified lactic acid bacterium of the invention (Example 2). A supplement to the RWP medium to provide a source of complex amino nitrogen was found essential, since fermentative growth and ethanol production were obtained when a supplement of yeast extract (0.5% w/v) was provided, while ammonium salts were insufficient (Example 2).

The use of YE as a supplement to RWP, while effective, does not provide a cost-effective growth medium for producing ethanol using lactic acid bacteria, for a number of reasons, as explained herein. Firstly, due to its high price (currently 7000˜10000 $/ton), it would increase the total ethanol production costs using the genetically modified lactic acid bacterium of the invention on RWP supplemented media by an estimated 30%, and for this reason alone there exists a need to find cheaper alternative sources of complex amino nitrogen.

Corn steep liquor having CAS Number: 66071-94-1 (CSL), which is a byproduct of the corn milling industry, is a cheaper amino nitrogen source that currently costs around 500 $/ton. CSL has a pH of about 4.0 and consists predominantly of naturally occurring nutritive materials such as water-soluble proteins, amino acids (e.g., alanine, arginine, aspartic acid, cysteine, glutamic acid, histidine, isoleucine, leucine, lysine, methionine, phenylalanine, proline, threonine, tyrosine, valine), vitamins (e.g., B-complex), carbohydrates, organic acids (e.g., lactic acid), minerals (e.g., Mg, P, K, Ca, S), enzymes and other nutrients. However, even though CSL is a source of complex amino nitrogen, when the genetically modified lactic acid bacterium of the invention was supplied with the RWP medium supplemented with CSL in an amount corresponding to 0.1% to 2.5% (w/v)), the final biomass concentration obtained was still very low and almost no ethanol was produced.

Surprisingly, the hydrolysis of CSL, for example acid hydrolysis, yielded a hydrolysed CSL composition (CSLH) that when used as a supplement to RWP provided an effective low cost medium for ethanol production by fermentation using the genetically modified lactic acid bacterium of the invention (Table 4, in Example 2). The need to use hydrolysed CSL as a supplement was unexpected, since many native strains of lactic acid bacteria, including the strain CS4435L used in the examples, comprise a cell-envelope bound protease enabling their growth on milk, which is also low in free amino acids and peptides. Only limited acid hydrolysis was required to increase the content of free peptides and amino acids in the CSL, sufficient to produce a suitable CSLH supplement. The growth and high yield of ethanol produced by the genetically modified lactic acid bacterium of the invention on RWP supplemented with the CSLH (obtained by limited acid hydrolysis) is consistent with the fact that cells of these lactic acid bacteria comprise both intracellular peptidases as well as various uptake systems for peptides as well as free amino acids that facilitate the assimilation of amino nitrogen in this form.

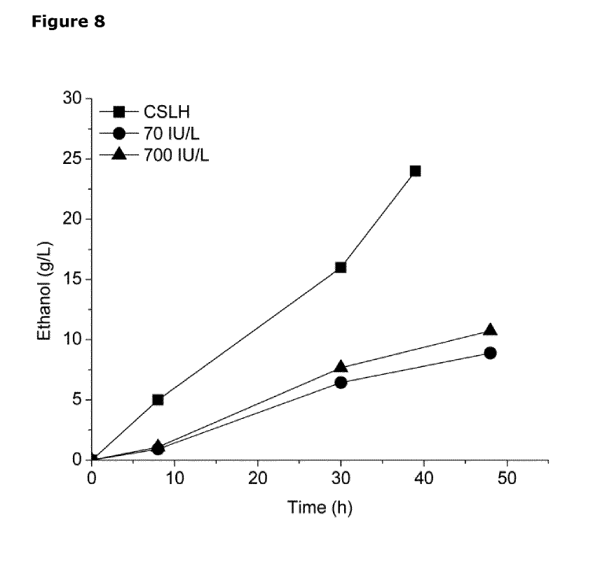

Alternative methods for improving growth and ethanol production were also tested using RWP supplemented with treated-forms of CSL (see example 4, FIG. 8). Surprisingly, growth media comprising acid-hydrolyzed CSLH supported significantly higher levels of ethanol production than proteinase-treated CSL. Thus, L. lactis strain CS4435L cultures grown in media supplemented with CSLH produced 24 g/L ethanol in 39 h; while cultures grown in media supplemented with 70 IU/L or 700 IU/L proteinase-treated CSL, only produced 8.9 g/L and 10.7 g/L ethanol in 48 h, respectively.

Down-stream processing of ethanol produced by fermentation, typically involves a distillation step. In order to minimize distillation costs it is important that the ethanol content of the fermentation broth is at least 4% (w/w) ethanol. Employing a fed-batch fermentation process, a fermentation broth comprising over 4.1% (w/w) ethanol was obtained when the genetically modified lactic acid bacterium of the invention was cultured on RWP supplemented with CSLH, and subsequent feeding with lactose (Example 3, and Table 4).

Accordingly, the invention provides a method for ethanol production employing a genetically engineered lactic acid bacterium comprising the steps of:

a. introducing a genetically modified lactic acid bacterium into an aqueous culture medium;
b. incubating the culture of (a);
c. recovering ethanol produced by said culture in step (b), and optionally
d. isolating the recovered ethanol;
wherein the aqueous culture medium comprises a lactose rich substrate (such as whey permeate or residual whey permeate), and a amino nitrogen source (such as acid hydrolysed corn steep liquor, as defined herein), and
wherein the genetically engineered lactic acid bacterium comprises transgenes encoding:
a polypeptide having pyruvate decarboxylase (PDC) activity (EC 4.1.1.1;
   and
a polypeptide having alcohol dehydrogenase B activity (EC 1.1.1.1);
   wherein the genome of said lactic acid bacterium is deleted for genes or lacks genes or lacks functional genes encoding polypeptides having an enzymatic activity of:
lactate dehydrogenase (E.C 1.1.1.27 or E.C. 1.1.1.28)
phosphotransacetylase (E.C. 2.3.1.8) and
bifunctional alcohol dehydrogenase E (E.C. 1.1.1.1 and EC 1.2.1.10); and wherein the the genome of said lactic acid bacterium comprises genes encoding polypeptides having:
lactose-specific phosphotransferase system (PTS) activity (EC 2.7.1.69)
phospho-β-D-galactosidase activity (EC 3.2.1.85)
galactose-6-phosphate isomerase activity (EC 5.3.1.26),
D-tagatose-6-phosphate kinase activity (EC 2.7.1.114), and
tagatose 1,6-diphosphate aldolase activity (EC 4.1.2.40);
and optionally a lactose transport regulator protein.

According to one embodiment of the method for ethanol production according to the invention, the aqueous culture medium comprises whey permeate or residual whey permeate, combined with acid hydrolysed corn steep liquor: wherein the final lactose content of the culture medium is at least 5, 10, 15, 20, 25, 30, 35, 40, 45, 50, 55, 60, 65, 70, 75, 80 g/L lactose; between any one of 2-200 g/L, 5-180 g/L, 5-150 g/L, 5-100 g/L, 10-100 g/L, 5-90 g/L, 10-90 g/L, 5-80 g/L, 10-80 g/L lactose; preferably equal to or less than any one of 150 g/L, 140 g/L, 130 g/L, 120 g/L, 110 g/L, 100 g/L, 90 g/L, and 80 g/L lactose. The desired lactose content of the aqueous culture medium is obtainable by diluting the whey permeate of residual whey permeate in the aqueous culture medium. For example, the aqueous culture medium may comprise any one of 1-80%, 5-80%, 10-80%, 20-80%, 20-60%, and 30-50% residual whey permeate.

The hydrolysed corn steep liquor (CSLH) component of the aqueous culture medium, is derived from corn steep liquor (CAS Number: 66071-94-1) by acid hydrolysis, for example hydrolysis with H₂SO₄, or HCl. The hydrolysed CSL is characterized by enhanced levels of soluble proteins, peptides and free amino acids (the concentrations of nearly all the 20 free amino acids is doubled compared with untreated corn steep liquor, especially arginine, glutamine, histidine, methionine, isoleucine, leucine, valine, and tyrosine. The peptides present in CSLH include oligopeptides of 2, 3, 4, 5 amino acid residues or even longer peptides; in particular the peptides Leu-Gly, Gly-Gly, Gly-Gly-Leu, Thr-Pro-Val-Gly-Lys.

A hydrolysed CSLH preparation, suitable for use as a supplement to a RWP medium, is one wherein the concentration of at least one free amino acid, selected from the group consisting on glutamine, histidine, methionine, leucine, isoleucine, and valine is at least 1.5 fold greater than the concentration of the corresponding amino acid in the original CSL from which the CSLH was derived by hydrolysis. Preferably, the concentration of the at least one free amino acid is at least 1.6, 1.7, 1.8, 1.9 or 2 fold greater than the concentration of the corresponding amino acid in the original CSL from which the CSLH was derived by hydrolysis. When the at least one free amino acid is histidine, the concentration of the histidine in a CSLH preparation having a 25% w/v solids content is at least 8 mM, preferably at least 9, 10, 11, 12, 13, 14, 15, 16, 17, 18, 19 or 20 mM; while the mM concentration of the histidine in a 2.5% (w/v solids content) CSLH preparation is correspondingly 10 folded lower. Alternatively, when the at least one free amino acid is histidine, the concentration of the histidine in a CSLH preparation having a 25% w/v solids content is at least 2 mM, preferably at least 2.5, 3.0, 3.5, 4.0, 4.5, 5.0, 5.5, 6.0 mM higher than the concentration of histidine in the original CSL from which the CSLH was derived by hydrolysis; while the mM concentration of the histidine in a 2.5% (w/v solids content) CSLH preparation is correspondingly 10 folded lower.

The RWP medium growth is supplemented with CSLH in an amount sufficient to support ethanol production using the genetically modified lactic acid bacterium of the invention. It has been observed that addition of acid hydrolysed treated CSL (H1) to give a final concentration of at least 0.5 w/v solids content was sufficient to enhance ethanol production and growth. Preferably, when the initial lactose concentration of the growth medium is between 5-80 g/L lactose, then the acid hydrolysed CSL (H1) is added to the medium in an amount to give a final w/v CSL solids concentration of at least 0.75, 1.00, 1.25, 1.50, 1.75, 2.00, 2.25, 2.5, 2.75, 3.0%. Preferably, when the initial lactose concentration of the growth medium is between 60-200 g/L lactose, then acid hydrolysed CSL (H1) is added in an amount to give a final CSL w/v solids concentration of at least 2.00, 2.25, 2.5, 2.75, 3.0, 3.25, 3.5, 3.75, 4.0, 4.25, 4.5, 4.75 and 5.0%. The desired final concentration of CSLH in the aqueous culture medium is obtainable by diluting the CSLH into the aqueous culture medium. CSL is obtainable in a CSL w/v solids concentration of 40 to 60%; typically 50%. Accordingly, a dilution of 10 fold into the aqueous culture medium will give a CSL w/v solids concentration of 4 to 6%; typically 5%. Since the solids content of CSL remains unaltered by the acid hydrolysis, the final dilution of CSLH required to obtain the desired final concentration are the same as those for the original CSL from which the CSLH was derived.

According to one embodiment of the method for ethanol production according to the invention, the aqueous culture medium may be further characterized as a composition consisting of the components: whey permeate of residual whey permeate (as defined herein); hydrolysed corn steep liquor (as defined herein); water and optionally supplemented with yeast extract and/or an aqueous solution of lactose.

The ethanol produced by fermentation using the genetically modified lactic acid bacterium of the invention can be recovered from the fermentation medium by steps including distillation.

### IV A Method of Detecting Ethanol Production

Methods for detecting and quantifying ethanol produced by a micro-organism of the invention include high performance liquid chromatography (HPLC) combined with Refractive Index detection to identify and quantify ethanol (as described by Solem et al., 2013) relative to a standard, as described and illustrated in the examples.

### V Methods for Producing a Lactic Acid Bacterium of the Invention

Integration and self-replicating vectors suitable for cloning and introducing one or more gene encoding one or more a polypeptide having an enzymatic activity required for homo-ethanol production in a lactic acid bacterium of the invention are commercially available and known to those skilled in the art (see, e.g., Sambrook et al., Molecular Cloning: A Laboratory Manual, Second Edition, Cold Spring Harbor Laboratory Press, 1989). Cells of a lactic acid bacterium are genetically engineered by the introduction into the cells of heterologous DNA (RNA). Heterologous expression of genes encoding one or more polypeptide having an enzymatic activity required for homo-ethanol production in a lactic acid bacterium of the invention is demonstrated in Example 1.

A nucleic acid molecule, that encodes one or more polypeptide having an enzymatic activity required for homo-ethanol production according to the invention, can be introduced into a cell or cells and optionally integrated into the host cell genome using methods and techniques that are standard in the art. For example, nucleic acid molecules can be introduced by standard protocols such as transformation including chemical transformation and electroporation, transduction, particle bombardment, etc. Expressing the nucleic acid molecule encoding the enzymes of the claimed invention may also be accomplished by integrating the nucleic acid molecule into the genome.

Deletion of endogenous genes in a host lactic acid bacterium to obtain a genetically modified lactic acid bacterium according to the invention can be achieved by a variety of methods; for example by transformation of the host cell with linear DNA fragments containing a locus for resistance to an antibiotic, or any other gene allowing for rapid phenotypic selection, flanked by sequences homologous to closely spaced regions on the cell chromosome on either side of the gene to be deleted, in combination with the immediate subsequent deletion or inactivation of the recA gene. By selecting for a double-crossover event between the homologous sequences, shown by the antibiotic resistance or other detectable phenotype, a chromosome disruption can be selected for which has effectively deleted an entire gene. Inactivation or deletion of the recA gene prevents recombination or incorporation of extrachromosomal elements from occurring, thereby resulting in a bacterial strain which is useful for screening for functional activity or production of genetically engineered proteins in the absence of specific contaminants. The deletion of the genes ldh, ldhB, ldhX, pta, and adhE from L. lactis given in Example 1, illustrates one method for deleting these genes.

The deletion of endogenous genes in a host lactic acid bacterium to obtain a genetically modified lactic acid bacterium according to the invention can also be achieved by the more traditional approach involving mutagenesis and screening/selection. For instance, LDH (lactate dehydrogenase) mutants can be screened out using solid medium containing 2,3,5-triphenyl tetrazolium following mutagenesis using for instance N-methyl-N′-nitro-N-nitrosoguanidine (NTG) or UV radiation. Alternatively, after mutagenesis, low-acid producing strains could be selected using a combination of bromide and bromate as described by Han et al., 2013. ALDB (α-acetolactate decarboxylase) mutants can be obtained easily after mutagenesis, for instance using NTG, or grown in the medium containing an unbalanced concentration of leucine versus valine and isoleucine in the medium (Goupil et al., 1996). ADHE (ethanol dehydrogenase) mutants can be screened in the presence of various concentrations of acetaldehyde.

### EXAMPLES

### Example 1 Genetic Modification of a Lactococcus lactis Strain for Production of Ethanol from Lactose

The genetic modifications required to produce a Lactococcus lactis strain that is capable of homo-ethanol production from lactose in dairy waste and to efficiently direct the flux towards this end product are described below.

### 1.1 Host Strains and Plasmids:

The plasmid-free strain Lactococcus lactis subsp. cremoris MG1363 or its derivatives were used for the studies described herein [18]. The Escherichia coli strain ABLE-C (E. coli C lac (LacZ−)[Kan^{r} McrA⁻ McrCB⁻ McrF⁻ Mrr⁻ HsdR (rₖ⁻ mₖ⁻)][F′ proAB lacI^{q}ZΔM15 Tn10(Tetr)]) (Stratagene) was used only for facilitating DNA cloning steps. The lactose-metabolism plasmid pLP712 (55,395 bp) was extracted from the dairy isolate NCDO712 based on the method of Andersen (1983).

### 1.2 Growth Conditions:

E. coli strains were grown aerobically at 30° C. in Luria-Bertani broth (Sambrook et al. 2001). For growth experiments L. lactis was grown in 100 ml flasks without shaking in defined SA medium (Jensen et al., 1993), where glucose was replaced by lactose, or residual whey permeate medium (RWP). RWP, which was provided from Arla Foods Ingredients Group P/S (http://www.arlafoodsingredients.com/), is the mother liquor from lactose production and its composition can be seen in Table 3. When required, yeast extract (Sigma-Aldrich, USA) was used as an amino nitrogen source. Antibiotics were added in the following concentrations: erythromycin: 200 μg/ml for E. coli and 5 μg/ml for L. lactis, tetracycline: 8 μg/ml for E. coli and 5 μg/ml for L. lactis, chloramphenicol: 20 μg/ml for E. coli and 5 μg/ml for L. lactis.

For nitrogen source optimization tests, 50 g/L lactose (diluted RWP) was mixed with different concentrations of nitrogen sources (NH₄Cl, yeast extract, CSL or CSLH) in 25 ml tube with a volume of 10 ml.

For ethanol production tests, RWP was diluted and used as the main substrate for fermentation without the addition of any vitamins or salts, except 2.5% (w/v) CSLH. L. lactis strain CS4435L was grown in a 125 ml flask with 100 ml of medium with slow magnetic stirring and no aeration. The cultivation was carried out at 30° C.

### 1.3 DNA Techniques:

All manipulations were performed according to Sambrook et al (1989). PCR primers used can be seen in Table 2. PfuX7 polymerase was used for PCR applications (Nørholm, 2010). Chromosomal DNA from L. lactis was isolated using the method described for E. coli with the modification that cells were treated with 20 μg of lysozyme per ml for 2 hours. Cells of E. coli were transformed using electroporation. L. lactis was made electro competent as described previously by Holo and Nes (1989), with the following modifications: the cells were grown with 1% glycine, and at an optical density of 0.5 (600 nm) ampicillin was added to a final concentration of 20 μg ml⁻¹ and incubation was continued at 30° C. for 30 minutes.

The plasmid vector pCS1966 (Solem et al., 2008) was used for deleting genes in L. lactis. Plasmids employed for deleting chromosomal genes were prepared by PCR amplifying approximately 800 base pairs (bp) regions upstream and downstream of the L. lactis chromosomal region to be deleted using the PCR primers and chromosomal DNA isolated from L. lactis. The primers used for amplifying the upstream and downstream regions are indicated in Table 2 as “geneX ups.” and geneX dwn”. The amplified fragments and the plasmid, pCS1966, were then digested with the respective restriction enzymes indicated in the primer table, prior to inserting the fragment into the plasmid. The resulting plasmids were transformed into the parent strain individually and gene deletion was performed as described by Solem et al., (2008). Specifically, the plasmids were transformed into the strains via electroporation, and the strains comprising the plasmids integrated into the chromosome were selected for on M17 plates supplemented with glucose and erythromycin. Afterwards, the transformants were purified and plated on SA glucose medium (Jensen et al., 1993) plates supplemented with 5-fluoroorotate, thereby selecting for strains in which the plasmid had been lost by homologous recombination. The successful deletions were verified by PCR (Solem et al., 2008).

1.4 Deleting Genes from the Lactococcus lactis Subsp. Cremoris

The following genes were deleted from the Lactococcus lactis subsp. cremoris parent strain ldhX, ldhB, ldh, pta, and adhE. The genes were deleted using gene deletion plasmids derived from pCS1966 designated as: pCS4026 (ldhX), pCS4020 (ldhB), pCS4104 (ldh), pCS4230 (pta), pCS4273 (adhE), constructed as described above (Example 1.2).

Deletion of the genes from the Lactococcus lactis subsp. cremoris parent strain was verified by PCR amplification of the respective gene using primers 774/777 (ldhX), 769/771 (ldhB), 788/789 (ldh), 880/881 (pta) and 929/930 (adhE).

The strain containing the three lactate dehydrogenase deletions (ldh, ldhB, ldhX) was named CS4099 or MG1363Δ3ldh. CS4234 (MG1363 Δ³ldhΔpta) was derived from CS4099, by additionally the deleting a phosphotransacetylase gene, pta using pCS4230. CS4363 (MG1363 Δ³ldhΔptaΔadhE) was derived from CS4234, by additionally the deleting the native adhE gene using pCS4273, using the gene deletion methods described in section 1.3.

TABLE 1
Strains and plasmids

| | | |
|---|---|---|
| Designation | Genotype or description | Reference |

L. lactis strains

| | | |
|---|---|---|
| MG1363 | Wild type | Gasson, 1983 |
| CS4099 | MG1363 Δ3ldh (Δldh, ΔldhB & ΔldhX) | Solem et al., |
| | | 2013 |
| CS4234 | MG1363 Δ3ldh Δpta | Solem et al., |
| | | 2013 |
| CS4363 | MG1363 Δ³ldh Δpta ΔadhE | Solem et al., |
| | | 2013 |
| CS4435 | MG1363 Δ³ldh Δpta ΔadhE pCS4268 | Solem et al., |
| | | 2013 |
| CS4435L | MG1363 Δ³ldh Δpta ΔadhE | This work |
| | pCS4268 pLP712 | |
| pG⁺host8 | E. coli/L. lactis shuttle vector, Tet^{R}, | Maguin et al., |
| | thermosensitive replicon | 1996 |
| pCS4268 | pG⁺host8::SP-ldh (L. lactis) | This work |
| pCS4564 | pG⁺host8::SP-ldhA (E. coli) | This work |
| pCI372 | E. coli/L. lactis shuttle vector, Cam^{R} | Hayes et al., |
| | | 1990 |
| pCS4518 | pCI372::gusA | This work |
| pLP712 | Lac plasmid from NCDO712 | Wegmann et al., |
| | | 2002 |

Abbreviations:
ldh (ldhB, ldhX), lactate dehydrogenase genes;
pta, phosphotransacetylase gene;
adhE: alcohol dehydrogenase genes;
pCS4268: pTD6-pdc-adhB (pdc, pyruvate decarboxylase gene from Zymomonas mobilis;
adhB, ethanol dehydrogenase gene from Z. mobilis).

1.5 Introducing Codon-Optimized Genes Encoding Pyruvate Decarboxylase and Alcohol Dehydrogenase B into L. lactis Strain MG1363 Δ³ldhΔptaΔadhE

The pyruvate decarboxylase gene (pdc) was amplified using primers 690/829 and the alcohol dehydrogenase gene (adhB) was amplified using 830/791. In both cases the templates were synthetic codon-optimized genes based on Zymomonas mobilis pdc and adhB genes (GenScript). An SP (synthetic promoter)-pdc-adhB fragment was amplified from CS4116 chromosomal DNA using primers 947/894, phosphorylated using T4 polynucleotide kinase and ligated to pTD6 amplified using 891/892. The ligation was transformed directly into CS4363 (MG1363 Δ3ldhΔptaΔadhE). CS4435 is a strain comprising the plasmid pCS4268 comprising and expressing the pdc-adhB genes. The plasmid pTD6 is a derivative of pAK80 (Solem et al., 2013) containing a gusA reporter gene. A PCR fragment was amplified from pAK80 using primers ptd45/ptd46. The gusA gene was amplified from E. coli MG1655 using primers ptd1/ptd2. After digesting both fragments with BglII and AvrII the fragments were ligated and transformed into E. coli strain MC1000. The resulting plasmid was named pTD5. The erythromycin marker of pTD5 was replaced by a tetracycline marker by PCR amplifying pTD5 using primers ptd49/ptd50, digesting the product with StuI/ApaI and ligating it with a StuI/ApaI fragment containing the tetracycline resistance genes amplified from pG+host8 (29) using primers ptd51/ptd52. The ligation was introduced in E. coli MC1000 and the result was plasmid pTD6.

TABLE 2
Primers

| Primer | | | SEQ ID |
|---|---|---|---|
| name | Primer use | Primer sequence (5′→3′) | No. |
| 43 | Verify insert in | AATTAACCCTCACTAAAGGG | 99 |
| (T3) | pCS1966 | | |
| 263 | 5′ end of gusA, rev. | CGCGATCCAGACTGAATG | 100 |
| 603 | Verify insert in | ATCAACCTTTGATACAAGGTTG | 101 |
| | pCS1966 | | |
| 690 | Modulate pdc | CTAGTCTAGACTGAACCCTACCGNNNNNAGTTTATT | 102 |
| | expression, XbaI | CTTGACANNNNNNNNNNNNNNTGRTATAATNNNNA | |
| | | AGTAATAAAATATTCGGAGGAATTTTGAAATGAGTT | |
| | | ATACAGTAGGAAC | |
| 691 | Used with 690, PstI | TCGGACTGCAGTTACAACAATTTATTTACAGGTTTTC | 103 |
| 702 | pLB85 | GAGTCGACCTGCAGAATTC | 104 |
| 703 | pLB85, XbaI | TCGACTCTAGAGGATCTAC | 105 |
| 768 | ldhB ups., PstI | AATTCCTGCAGCATATTAAATAATGAACAAGTCATT | 106 |
| | | C | |
| 769 | ldhB ups., BamHI | TAGTGGATCCTGGTAAATCCAAACACAACAAC | 107 |
| 770 | ldhB dwn., PstI | AATTCCTGCAGTAATTTCCAGCTCTTACAATAAC | 108 |
| 771 | ldhB dwn., XhoI | GACCTCGAGTCAGAAACTTTCTTTACCAGAG | 109 |
| 772 | pCS1966, BamHI | GCGGGGATCCACTAGTTCTAG | 110 |
| 773 | pCS1966, XhoI | ATACCGTCGACCTCGAG | 111 |
| 774 | ldhX ups., BamHI | TAGTGGATCCCTGTTTCAGGTCTTGGATAG | 112 |
| 775 | ldhX ups., EcoRI | CCGATGAATTCTCATTAGCACGTTTAACAAGAG | 113 |
| 776 | ldhX dwn., EcoRI | CCGATGAATTCATCAGCGTAGTCTGCTGC | 114 |
| 777 | ldhX dwn., KpnI | CGGGGTACCATTTAATCCTAAAGTCGTTATTAC | 115 |
| 785 | ldh ups., EcoRI | CCGATGAATTCTTAAGTCAAGACAACGAGGTC | 116 |
| 786 | ldh dwn, EcoRI | CCGATGAATTCGACCTTGTTGAAAAAAATCTTC | 117 |
| 787 | ldh ups., BamHI | TAGTGGATCCGTACAATGGCTACTGTTAAC | 118 |
| 788 | ldh dwn., XhoI | GACCTCGAGGATGAACAGACTTTTTTATTATAG | 119 |
| 789 | Verify ldh deletion | AAAACCAGGTGAAACTCGTC | 120 |
| 791 | adhB rev, PstI | TCGGACTGCAGTTAAAATGCTGATAAAAACAATTCT | 121 |
| | | TC | |
| 827 | pCS1966, BamHI | ATACCGTCGACCTCGAG | 122 |
| 828 | pCS1966, XhoI | CGATAAGCTTGATATCGAATTC | 123 |
| 829 | Used with 690, | CCGATGAATTCTTACAACAATTTATTTACAGGTTTTC | 124 |
| | EcoRI | | |
| 830 | adhB fwd, EcoRI | CCGATGAATTCTATAAGGAGAATTAGAATGGCAAGT | 125 |
| | | AGTACATTTT ATATTC | |
| 834 | pfl ups., XbaI | CTAGTCTAGACAAGTGATGTACCAAATGAC | 126 |
| 835 | pfl ups., BamHI | CGCGGATCCTTTGAAATCTCCTTTGTTCT | 127 |
| 836 | pfl dwn., BamHI | CGCGGATCCTTCTTAGTATTAAAAAATATAAAG | 128 |
| 837 | pfl dwn., XhoI | GGTACTCGAGTGTGATTCACCCCTATTTCT | 129 |
| 845 | Synthetic promoter | CTAGTCTAGACTGAACCCTACCGTGGGGAGTTTATT | 130 |
| | primer, pdc, XbaI | CTTGACAAGTTCTCTTGGAGTTGATATAATCAAGAA | |
| | | GTAATAAAATATTCGGAGGAATTTTG | |
| 846 | Synthetic promoter | CTAGTCTAGACTGAACCCTACCGGTCAGAGTTTATT | 131 |
| | primer, pdc, XbaI | CTTGACATCGTTACCGTAGGTTGGTATAATCTTGAA | |
| | | GTAATAAAATATTCGGAGGAATTTTG | |
| 847 | Synthetic promoter | CTAGTCTAGACTGAACCCTACCGGCAAGAGTTTATT | 132 |
| | primer, pdc, XbaI | CTTGACAATTACCTGTTCGCTTGATATAATAAGGAA | |
| | | GTAATAAAATATTCGGAGGAATTTTG | |
| 848 | Synthetic promoter | CTAGTCTAGACTGAACCCTACCGGTCGAAGTTTATT | 133 |
| | primer, pdc, XbaI | CTTGACAATCTGTGAGATCAGTGATATAATACAGAA | |
| | | GTAATAAAATATTCGGAGGAATTTTG | |
| 878 | pta ups., USER | ATCCCTCGGTTACAAGTTTCU | 134 |
| 879 | pta dwn., USER | AGAAACTTGTAACCGAGGGAUAATAATAGATTGAA | 135 |
| | | ATTCTGTCAG | |
| 880 | pta ups., USER | ATTCGATATCAAGCTTATCGAUCAAAAATTGTGGTA | 136 |
| | | GAATATATAG | |
| 881 | pta dwn., USER | AGGTCGACGGTATCGATAAUCCTAGTTCAATTGATG | 137 |
| | | TGAC | |
| 882 | pCS1966, USER | ATCGATAAGCTTGATATCGAAU | 138 |
| 883 | pCS1966, USER | ATTATCGATACCGTCGACCU | 139 |
| 891 | pTD6, USER | ACAGATTAAAGGTTGACCAGTAU | 140 |
| 892 | pTD6, USER | ACCAATTCTGTGTTGCGCAU | 141 |
| 894 | adhB rev., USER | ATACTGGTCAACCTTTAATCTGUTTAAAATGCTGAT | 142 |
| | | AAAAACAATTCTT | |
| 920 | pCS1966, USER | ATAAGCTUGATATCGAATTCCT | 143 |
| 921 | pCS1966, USER | ATTCCCTTUAGTGAGGGTTAAT | 144 |
| 927 | adhE ups., USER | ATGTGTACGUTCTCCTTTGTG | 145 |
| 928 | adhE dwn., USER | ACGTACACAUATTATAGTATTTGGAACCGAAC | 146 |
| 929 | adhE ups., USER | AAGCTTAUGGTCGTCTTGTTACTTGTG | 147 |
| 930 | adhE dwn., USER | AAAGGGAAUTCTGCCGGAGCTATATATG | 148 |
| 947 | Modulate pdc | ATGCGCAACACAGAATTGGUGGCCNNNNNAGTTTA | 149 |
| | expression, USER | TTCYYRAMANNNNNNNNNNNNNNTGRYAYAAYNN | |
| | | NNAAGTAATAAAATATTCGGAGGAAT | |
| ptd1 | gusA fwd. | GACTGAACTAGTAGATCTGCAGAAGCTTGTCGACCC | 150 |
| | | GGGTACCTCGAGCTCCATGGCATATGCGGCCGCATG | |
| | | CGCAACACAGAATTGGTTAAC | |
| ptd2 | gusA rev. | ACCTCTCCTAGGATTAAAAAATAAAAAAGAACCCA | 151 |
| | | CTCGGGTTCTTTTTTTTACTAGCTAGCTAATGGTGCG | |
| | | CCAGGAGAGTTG | |
| ptd45 | pAK80 | TTCTGCAGATCTTCAGTCAGTCAAGAGGTTTGATGA | 152 |
| | | CTTTGAC | |
| ptd46 | pAK80 | TTTAATCCTAGGGATTGTGGGAAATTTAGGCG | 153 |
| ptd49 | pTD5, StuI | ATGCTAGGGCCCACGGGGAATTTGTATCGATG | 154 |
| ptd50 | pTD5, ApaI | TGATAAAGGCCTTACTGCACTATCAACACACT | 155 |
| ptd51 | pG⁺host8, StuI | TTAAGAAGGCCTAAGAAATTTCGCCAGTCG | 156 |
| Ptd52 | pG⁺host8, ApaI | GCGCCTGGGCCCGCCACTCATAGTTCTAAAC | 157 |

1.6 Introducing Genes Encoding the Lactose Catabolism Pathway into Lactococcus lactis Subsp. Cremoris Strain MG1363 Δ³LdhΔPtaΔadhE

The wild type strain L. lactis MG1363, and its derivatives described herein (e.g. CS4435), are plasmid-free strains that cannot utilize lactose as a carbon source. The Lactococcus plasmid, pLP712 (55.395 kbp), comprises genes encoding the entire lactose catabolism pathway (Wegmann et al., 2012). The lactose-metabolism plasmid pLP712 (55,395 bp) was extracted from the dairy isolate NCDO712 based on the method of Andersen (1983); and then transformed into L. lactis strain CS4435 to give strain CS4435L.

The lactose catabolism pathway genes located on the pLP712 plasmid (FIG. 2) are as follows:

1. the lacAB genes encoding two subunit polypeptides that together have galactose-6-phosphate isomerase activity (EC 5.3.1.26); wherein the first subunit polypeptide has an amino acid sequence of SEQ ID NO: 90 encoded by the L. lactis lacA gene; and the second subunit polypeptide has an amino acid sequence of SEQ ID NO: 92 encoded by the L. lactis lacB gene;
2. the lacC gene encoding a polypeptide having D-tagatose-6-phosphate kinase activity (EC 2.7.1.114); wherein the polypeptide has an amino acid sequence of SEQ ID NO: 94);
3. the lacD gene encoding a polypeptide having tagatose 1,6-diphosphate aldolase activity (EC 4.1.2.40); wherein the polypeptide has an amino acid of SEQ ID NO: 96;
4. the lacEF genes encoding a two polypeptide components together having lactose-specific phosphotransferase system (PTS) activity (EC 2.7.1.69); wherein the first polypeptide component is a phosphotransferase system EIICB component having an amino acid sequence of SEQ ID NO: 84, encoded by the L. lactis lacE gene; and the second polypeptide component is a phosphotransferase system EIIA component having an amino acid sequence of SEQ ID NO: 86, encoded by the L. lactis lacF gene; and
5. the lacG gene encoding a polypeptide having phospho-β-D-galactosidase activity (EC 3.2.1.85); wherein the polypeptide has the amino acid sequence of the phospho-β-D-galactosidase of SEQ ID NO: 88; and
6. the lacR gene encoding a lactose transport regulator of SEQ ID NO: 98

### 1.7 Fermentation Properties of the Lactic Acid Bacterium Genetically Modified for Ethanol Production

The wild type strain MG1363 and its genetically modified derivative strains, as listed in Table 3, were cultivated in defined SA medium (Jensen et al., 1993) with glucose and samples of each culture were collected after 24 hours. Cell growth was regularly measured by OD⁶⁰⁰ and quantification of lactose, glucose, lactate, formate, acetate, ethanol, acetoin and 2,3-butanediol was carried out using an Ultimate 3000 high-pressure liquid chromatography system (Dionex, Sunnyvale, USA) equipped with a Aminex HPX-87H column (Bio-Rad, Hercules, USA) and a Shodex RI-101 detector (Showa Denko K.K., Tokyo, Japan). The column oven temperature was set at 60° C. and the mobile phase of consisted of 5 mM H₂SO₄, at a flow rate of 0.5 ml/min. CO₂ is included for calculating the carbon balance. Values are averages of three independent experiments and standard deviations are indicated.

Lactococcus lactis is characterised by homolactic fermentation when grown on glucose, as illustrated for the wild-type strain MG1363 in Table 3. Following deletion of the three ldh genes, in strain CS4099, the dominant products become acetate and ethanol, while the production of acetate was eliminated by deleting the pta gene, in strain CS4234, however this strain still produced significant amounts of formate and acetoin, and small amounts of 2,3-butandiol. The additional deletion of the native adhE gene yielded strain CS4363 that was unable to grow under anaerobic conditions because of the defect in cofactor regeneration ability. However, its growth could be restored in the presence of O₂, where NAD⁺ is recycled by NADH oxidase (NoxE) which results in acetoin as the main fermentation product. The introduction of pdc and adhB genes, however, creating strain CS4435, restored complete cofactor-recycling and bacterial growth under anaerobic conditions, and the outcome was a genetically modified L. lactis strain capable of homo-ethanol production from glucose as carbon source (Table 3). The native adhE gene encodes a bifunctional ADHE that can use both acetyl-CoA and acetaldehyde as substrate, while the transgene adhB, encoding an ADHB derived from from Z. mobilis only uses acetaldehyde. As a consequence, the cofactors are completely balanced in the heterologous ethanol forming pathway and nearly all the carbon balance is converted to ethanol.

TABLE 3
Fermentation products of wild type and genetically modified L. lactis

| | Carbon | Lactate | Acetate | Formate | Acetoin | 2,3-Butanediol | Ethanol |
|---|---|---|---|---|---|---|---|
| Strains | balance | mol/mol glucose | | | | | |
| MG1363 | 90% | 1.8 ± 0.1 | ND | ND | ND | ND | ND |
| CS4099 | 85% | ND | 0.61 ± 0.02 | 1.58 ± 0.07 | 0.08 ± 0.01 | 0.09 ± 0.01 | 0.65 ± 0.02 |
| CS4234 | 88% | ND | ND | 0.82 ± 0.05 | 0.35 ± 0.02 | 0.16 ± 0.03 | 0.76 ± 0.04 |
| CS4363 | 85% | ND | ND | ND | 0.85 ± 0.05 | ND | ND |
| CS4435 | 87% | ND | ND | ND | ND | ND | 1.75 ± 0.06 |

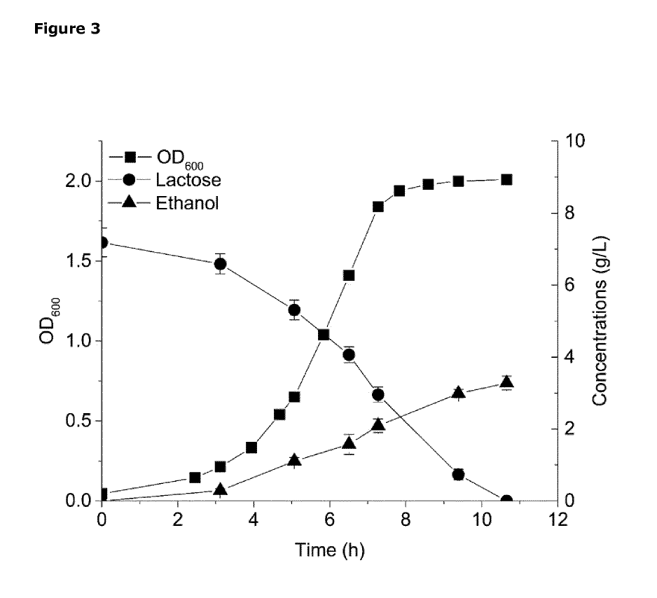

The strain CS4435L, derived from strain CS4435, comprises the Lactococcal plasmid, pLP712 (55.395 kbp) that encodes the entire lactose catabolism pathway (FIG. 2). Strain CS4435L was grown on a defined SA medium (Jensen et al., 1993) comprising 7.2 g/L lactose as the sole carbon source. As seen in FIG. 3, the cells grew and consumed the supplied lactose completely within 11 hours and the only fermentation product was ethanol. The measured growth rate was 0.6 h⁻¹, close to the growth rate on glucose (0.7 h⁻¹). The ethanol concentration reached during cultivation of strain CS4435L was 3.2 g/L, with a yield of 0.45 g ethanol/g lactose, corresponding to 83% of the theoretical maximum. The final ethanol titer was increased to 6.7, 10.3 and 12.0 g/L when the initial lactose concentration was increased to 15.1, 24.0 and 31.8 g/L, respectively (Table 2).

TABLE 4
Optimization of lactose concentrations in different types of media.

| | | | | Yield (g ethanol/g | Conversion |
|---|---|---|---|---|---|
| Lactose (g/L) | Media types | OD₆₀₀ | Ethanol (g/L) | lactose) | efficiency^{a} |
| 7.2 | SA^{b} | 2.0 | 3.2 | 0.45 | 0.83 |
| 15.1 | SA^{b} | 2.3 | 6.7 | 0.44 | 0.82 |
| 24.0 | SA^{b} | 2.9 | 10.3 | 0.43 | 0.80 |
| 31.8 | SA^{b} | 3.6 | 12.0 | 0.38 | 0.71 |
| 32.0 | RWP + YE^{c} | 7.2 | 13.4 | 0.42 | 0.78 |
| 40.0 | RWP + CSLH^{d} | 4.0 | 17.5 | 0.44 | 0.82 |
| 80.0 | RWP + CSLH^{e} | 6.0 | 30.6 | 0.40 | 0.71 |
| 80.0^{m} | RWP + CSLH^{f} | 6.0 | 41.0 | 0.38 | 0.70 |

^{a}= Conversion efficiency was calculated based on the theoretical maximal yield (0.538 ethanol/g lactose).
^{b}= The composition of SA medium (Jensen et al. 1993) comprises 19 amino acids, vitamins and salts. Glucose was replaced by lactose.
^{c}= Diluted RWP (residual whey permeate) and 0.5% (w/v) YE (yeast extract).
^{d-f}= Diluted RWP (residual whey permeate) and 2.5% (w/v) CSLH (corn steep liquor hydrolysate), prepared by condition H1.
^{m}= Fed-batch was performed with initial 80 g/L lactose and the details can be found in FIG. 7.

### Example 2 Development of a Low Cost Medium for Production of Ethanol from Lactose by Genetically Modified Lactococcus lactis Strain of the Invention

Waste stream residual whey permeate (RWP) is the permeate mother liquor after extracting lactose from whey permeate. The composition of the RWP, which was supplied by Arla Foods Ingredients Group P/S (http://www.arlafoodsingredients.com) was determined and shown in Table 5. The sugar components of a filtered sample of RWP were determined as described in example 1.6, and the amino acid composition was determined by the steps of hydrolysis of the filtered sample with 6 M HCl; separation by ion exchange chromatography and detection after oxidation and derivatization with o-phthaldialdehyde, as described by Barkholt et al., (1989).

TABLE 5
The composition of residual whey permeate^{a}

| Composition | Concentration | |
|---|---|---|
| Lactose | 150 | g/L |
| Galactose | 3 | g/L |
| Aspartate | 0.252 mM (mmol/L) | |
| Threonine | 0.076 | mM |
| Serine | 0.088 | mM |
| Glutamate | 1.464 | mM |
| Proline | 0.384 | mM |
| Glycine | 0.904 | mM |
| Alanine | 0.24 | mM |
| Cysteine | 0.096 | mM |
| Valine | 0.072 | mM |
| Methionine | 0.124 | mM |
| isoleucine | 0.04 | mM |
| Leucine | 0.092 | mM |
| Histidine | 0.208 | mM |
| Lysine | 0.304 | mM |
| Arginine | 0.096 | mM |

^{a}Residual whey permeate is a concentrate of the residue remaining after lactose extraction from whey permeate.

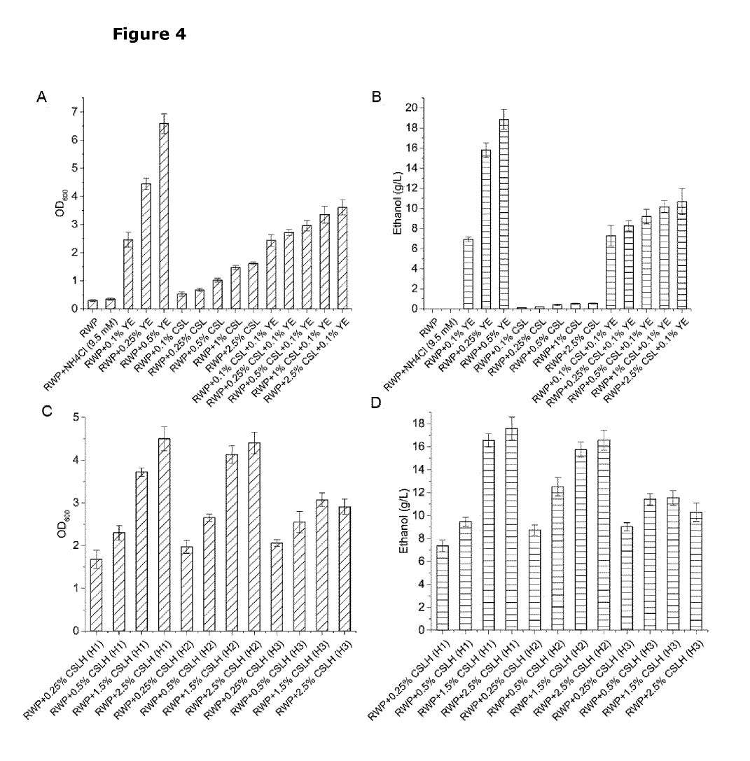

Although strain CS4435L both grew and produced ethanol when cultured on defined SA medium (Jensen et al., 1993) comprising 7.2 g/L lactose; the strain was unable to grow or produce ethanol formation when cultured on RWP alone, even though an initial the lactose content of 50 g/L lactose was provided (FIG. 4A-B). In view of the relatively low amino acid content of RWP, various nitrogen supplements, added at different concentrations to the RWP, were tested to support growth and ethanol production of strain CS4435L. As seen in FIG. 4, CS4435L was unable to grow in RWP supplemented with inorganic NH₄Cl. However, the addition of yeast extract (YE) enhanced growth; and after 12 hours fermentation on a RWP medium comprising 0.5% (w/v) YE, the culture reached a final cell density (OD600ₙₘ) of 6.5, and a final ethanol concentration was nearly 19 g/L. Accordingly, growth and ethanol production CS4435L on a RWP medium requires the addition of a complex nitrogen source.

Since the price of YE is too high to provide the basis for a low cost medium; corn steep liquor (CSL) was tested as a cheap source of complex nitrogen. CSL was purchased from Sigma-Aldrich (St. Louis, Mo.) with 40-60% solid content. RWP medium supplemented with CSL, at concentrations ranging from 0.1% to 2.5% (w/v), however, only supported low levels of cell growth of strain CS4435L, and levels of ethanol produced were also very low. A combination of 0.1% (w/v) YE with CSL, led to only a small stimulation of growth (FIG. 4A-B).

In order to enhance the available amino nitrogen content of CSL, samples of CSL were subjected to various degrees of acid hydrolysis. The acid hydrolysis was performed with very small amounts of sulfuric acid (0.05-0.5% concentrated H₂SO₄ added to CSL having a 20-30% w/v solid content). The following hydrolysis conditions were applied to produce corn steep liquor hydrolysates (CSLH). H1 condition: original CSL was diluted 2 times with water and then 50 μl concentrated sulfuric acid was mixed with 100 ml diluted CSL. The mixture was kept at 121° C. for 15 mins and subsequently the pH was adjusted to 6.8-7.1 with the addition of 10 M NaOH solution. H2 condition: original CSL was diluted 2 times with water and then 250 μl concentrated sulfuric acid was mixed with 100 ml diluted CSL. The mixture was kept at 121° C. for 15 mins and subsequently the pH was adjusted to 6.8-7.1 with the addition of 10 M NaOH solution. H3 condition: original CSL was diluted 2 times with water and then 500 μl concentrated sulfuric acid was mixed with 100 ml diluted CSL. The mixture was kept at 121° C. for 15 mins and subsequently pH was adjusted to 6.8-7.1 with the addition of 10 M NaOH solution.

When comparing the growth and ethanol production of CS4435L cultured on RWP supplemented with the CSL hydrolysates (CSLH), the use of H1 hydrolysate (CSL treated with 0.05% H₂SO₄) gave the greatest increase in production levels (FIG. 4C-D). Increasing the amount of sulfuric acid used during acid hydrolysis (H2, 0.25% H₂SO₄) did not further improve the ability of the CSLH to support cell growth and ethanol production, and even had a negative effect at the highest acid concentration tested (H3, 0.5% H₂SO₄). Thus, a cell density of only 3.0 (OD600ₙₘ) and the final ethanol titer of only 11 g/L was obtained when using 2.5% (w/v) CSLH.

Analysis of the free amino acid composition of CSL revealed that hydrolysis of corn steep liquor increases the free amino acid content of CSL by circa 2 fold in comparison with untreated corn steep liquor.

TABLE 6
Free amino acid composition of CSL before and after hydrolysis

| Amino acids | CSL | Hydrolyzed CSL (H1) |
|---|---|---|
| Unit (mM) | 25% (w/v) | 25% (w/v) |
| Aspartate | 1.8 | 3.6 |
| Glutamate | 0.9 | 2 |
| Asparagine | 1.4 | 3 |
| Glutamine | 0.8 | 2.1 |
| Histidine | 6.1 | 11 |
| Arginine | 4.5 | 10.5 |
| Alanine | 2.1 | 5 |
| Tyrosine | 0.8 | 2.1 |
| Cysteine | 3.7 | 5.7 |
| Valine | 2.1 | 4.8 |
| Isoleucine | 1.8 | 3.6 |
| Leucine | 0.8 | 2.5 |
| Methionine | 0.1 | 1.2 |

A direct correlation was observed between the amount of CSLH added to the RWP medium and the final cell biomass and ethanol produced (FIG. 4C-D). When the RWP was supplemented with 2.5% (w/v) corn steep liquor hydrolysate (CSLH), a high final cell density of 4.5 (OD600ₙₘ) and a high ethanol titer (17.5 g/L) were obtained after 30 hours of fermentation from 50 g/L lactose. The ethanol yield obtained with CS4435L was only marginally less than that achieved when cells were grown on RWP supplemented with 0.5% (w/v) YE; although cell growth was reduced compared to the YE supplemented media.

The composition of two tested low cost growth media composed of RWP supplemented with hydrolysed CSL is shown in Table 7.

TABLE 7
Composition of two initial growth media comprising

| | Low lactose Medium 1 | | High lactose Medium 2 | |
|---|---|---|---|---|
| | RWP | CSLH | RWP | CSLH |
| | 50 g/L | (H1) | 80 g/L | (H1) |
| Composition | lactose | 2.5% w/v | lactose | 5% w/v |
| Lactose (g/L) | 50 g/L | | 80 g/L | |
| Galactose (g/L) | 1 g/L | | 1.6 g/L | |
| Aspartate (mM) | 0.08 | 0.36 | 0.135 | 0.72 |
| Threonine (mM) | 0.025 | | 0.040 | |
| Serine (mM) | 0.003 | | 0.047 | |
| Glutamate (mM) | 0.488 | 0.20 | 0.787 | 0.40 |
| Proline (mM) | 0.012 | | 0.206 | |
| Glycine (mM) | 0.301 | | 0.480 | |
| Alanine (mM) | 0.08 | 0.50 | 0.129 | 1.00 |
| Cysteine (mM) | 0.032 | 0.57 | 0.051 | 1.10 |
| Valine (mM) | 0.024 | 0.48 | 0.038 | 0.96 |
| Methionine (mM) | 0.041 | 0.12 | 0.066 | 0.24 |
| Isoleucine (mM) | 0.04 | 0.36 | 0.021 | 0.72 |
| Leucine (mM) | 0.092 | 0.25 | 0.049 | 0.50 |
| Histidine (mM) | 0.069 | 1.10 | 0.112 | 2.20 |
| Lysine (mM) | 0.101 | | 0.163 | |
| Arginine (mM) | 0.032 | 0.30 | 0.051 | 0.60 |
| Glutamine (mM) | | 0.20 | | 0.40 |
| Tyrosine (mM) | | 0.21 | | 0.42 |

The low cost growth medium composed of RWP, used in an amount conferring a lactose concentration of either 50 g/L or 80 g/L when supplemented with 2.5% or 5% CSLH (H1) respectively, provides lactose as substrate for ethanol production and amino acids in sufficient amounts to support ethanol production and growth. The 2.5% or 5% CSLH, hydrolysed under the conditions of H1 is sufficient to enhance the levels of available complex amino nitrogen (soluble polypeptides, peptides and free amino acids), and in particular the levels of the essential amino acids glutamine, histidine, methionine, leucine, isoleucine, and valine.

### Example 3 Optimizing Ethanol Production by Genetically Modified Lactococcus lactis Strain of the Invention on a Low Cost Medium

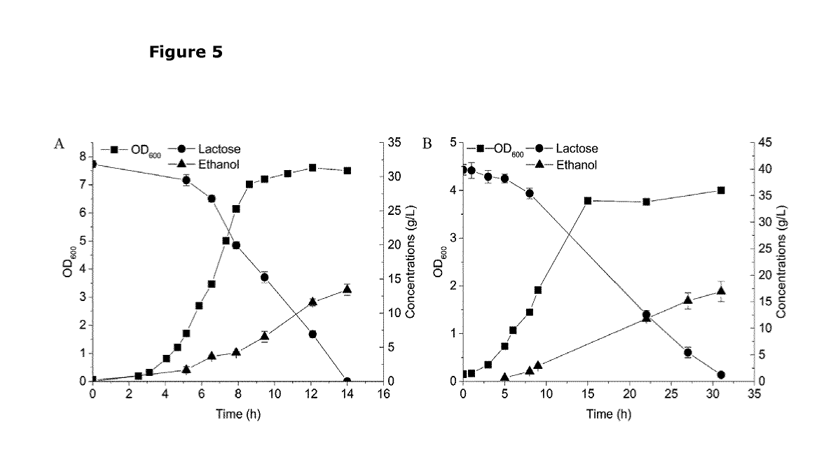

Ethanol production by L. lactis strain CS4435L and cell growth during fermentation on the low cost medium, RWP supplemented with 2.5% (w/v) CSLH (H1), was monitored in order to determine the yields obtainable by this process. Cell growth reached a final cell density (OD600ₙₘ) of 4.0 after 14.5 hours in medium with 2.5% (w/v) CSLH (H1); and the initial lactose content of 40 g/L lactose was completely consumed within 31 hours (FIG. 5B). The ethanol concentration increased linearly to 17.5 g/L, corresponding to 82% of the theoretical maximum (Table 4). By comparison, fermentation RWP supplemented with 0.5% (w/v) YE (FIG. 5A), gave a lower ethanol yield of 13.4 g/L, corresponding to 78% of the theoretical maximum (Table 4), while cell growth was faster and the final cell density was higher (OD600ₙₘ=7.2). Thus, the replacement of YE with cheap CSLH as a supplement to the RWP medium resulted in a 44% decrease in cell biomass yield, but an overall increase in ethanol production yield.

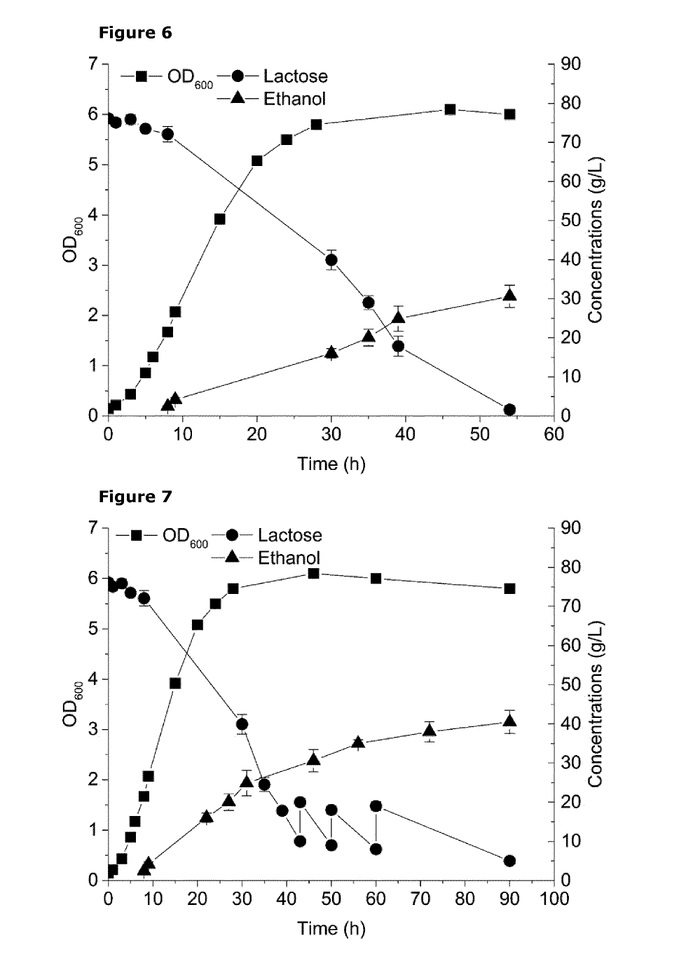

Enhanced ethanol titers obtainable with L. lactis strain CS4435L, during fermentation on the low cost medium, RWP supplemented with 2.5% (w/v) CSLH (H1), were achieved by raising the initial lactose content of the medium. When the initial lactose content of the medium was raised to 80 g/L lactose, the lactose was totally consumed after 55 hours fermentation (FIG. 6), where the ethanol titer reached 30.6 g/L, corresponding to a yield of 71% (Table 4). However, even higher initial lactose content of the medium provided at the beginning of the fermentation failed to further enhance the ethanol titer.

In order to enhance lactose supply during fermentation, without further elevating the lactose concentration in the low cost medium, fed-batch fermentation method was implemented, whereby lactose was added during fermentation. Specifically, fed-batch was performed with diluted RWP comprising a lactose content of 80 g/L lactose supplemented with 2.5% (w/v) CSLH; and 500 g/L lactose stock solution was used for feeding. The feeding was performed when the lactose concentration was lower than 10 g/L and after rapid injection it returned to around 20 g/L.

Using this fermentation strategy using L. lactis strain CS4435L on the low cost medium (FIG. 7), gave a final titer of 41 g/L ethanol after nearly 90 hours, corresponding to a yield of 70% (Table 4). The achieved ethanol titer of 5.2% meets the requirements for subsequent low cost ethanol distillation.

### Example 4 Treatment of CSL as a Source of Amino Nitrogen for a Genetically Modified Lactococcus lactis Strain

In order for L. lactis strain CS4435L to grow and produce ethanol when cultured on diluted residual whey permeate medium and 80 g/L lactose; it requires a source of amino nitrogen (Example 2). Example 2 further shows that CSL can only serve as a cheap source of amino nitrogen when it is hydrolysed. The present example compares three different methods of hydrolysing CSL included in the growth medium for L. lactis strain CS4435L, and their respective impacts on ethanol production. The CSI was either hydrolysed with acid or it was proteolytically hydrolysed. FIG. 8 shows ethanol production over time by a L. lactis strain CS4435L when cultured on diluted residual whey permeate medium and 80 g/L lactose supplemented with one of: (1) 2.5% (w/v) CSLH prepared under H1 conditions (CSL treated with 0.05% H₂SO₄ as described in Example 2); (2) 2.5% (w/v) CSL and 70 IU/L proteinase; and (3) 2.5% (w/v) CSL and 700 IU/L proteinase (where the protease was added to the growth medium prior to cultivation. Samples were collected periodically for determining ethanol concentration (filled triangles), over a period of 40-50 hours as displayed. The proteinase was derived from Aspergillus melleus Type XXIII (P4032 supplied by Sigma).

Surprisingly, growth media comprising acid hydrolysed CSLH supported significantly higher levels of ethanol production than proteinase-treated CSL. Thus, L. lactis strain CS4435L cultures grown in media supplemented with CSLH produced 24 g/L ethanol in 39 h; while cultures grown in media supplemented with 70 IU/L or 700 IU/L proteinase-treated CSL, only produced 8.9 g/L and 10.7 g/L ethanol in 48 h, respectively.

### REFERENCES

1. Anderson D. (1983) Simple and Rapid Method for Isolating Large Plasmid Dna From Lactic Strepococci. Appl Environ Microbiol.; 46:549-552.
2. Barkholt V, Jensen A L., (1989) Amino acid analysis: determination of cysteine plus half-cystine in proteins after hydrochloric acid hydrolysis with a disulfide compound as additive. Anal Biochem., 177:318-322.
3. Gasson M J. (1983) Plasmid complements of Streptococcus lactis NCDO 712 and other lactic streptococci after protoplast-induced curing. J Bacteriol., 154:1-9.
4. Goupil, N., Corthier, G., Ehrlich, S. D., Renault, P. Imbalance of leucine flux in Lactococcus lactis and its uses for the isolation of diacetyl-overproducing strains. Appl. Environ.Microbiol. 62, 2636-2640 (1996).
5. Han, S.-H., Lee, J.-E., Park, K., Park, Y.-C. Production of 2,3-butanediol by a low-acid producing Klebsiella oxytoca NBRF4. New Biotechnology 30, 166-172 (2013).
6. Holo H, Nes I. (1989) High-frequency transformation, by electroporation, of Lactococcus lactis subsp. Cremoris grown with glycine in osmotically stabilized media. Appl Environ Microbiol., 55:3119-3123.
7. Jensen P, Hammer K. (1993) Minimal requirements for exponential growth of Lactococcus lactis. Appl Environ Microbiol. 59:4363-4366.
8. Ling K. Charles (2008) Whey to Ethanol: A Biofuel Role for Dairy Cooperatives? Rural Business and Cooperative Programs Research Report 214 (http://www.rd.usda.gov/files/RR214.pdf)
9. Maguin, E., Prevost, H., Ehrlich, S. D. and Gruss, A. (1996) Efficient insertional mutagenesis in lactococci and other Gram-positive bacteria. J Bacteriol 178, 931-935.
10. Nørholm MHH (2010) A mutant Pfu DNA polymerase designed for advanced uracil-excision DNA engineering BMC Biotechnol., 10:21.
11. Sambrook J, Russell D. (2001) Molecular Cloning: A Laboratory Manual. Cold Spring Harbor Laboratory Press.
12. Solem, C., Dehli, T. & Jensen, P.R. (2013) Rewiring Lactococcus lactis for ethanol production. Appl. Environ. Microbiol. 79, 2512-2518.
13. Wegmann U, Overweg K, Jeanson S, Gasson M, Shearman C. (2012) Molecular characterization and structural instability of the industrially important composite metabolic plasmid pLP712. Microbiol., 158:2936-2945

1. A method for ethanol production using a genetically engineered lactic acid bacterium comprising the steps of:
a. introducing a genetically modified lactic acid bacterium into an aqueous culture medium;
b. incubating the culture of (a);
c. recovering ethanol produced by said culture during step (b), and optionally
d. isolating the recovered ethanol;
wherein the aqueous culture medium comprises:
I. whey permeate or residual whey permeate, and
II. an amino nitrogen source, and
wherein the genetically engineered lactic acid bacterium comprises transgenes encoding:
i. a polypeptide having pyruvate decarboxylase (PDC) activity (EC 4.1.1.1); and
ii. a polypeptide having alcohol dehydrogenase B activity (EC 1.1.1.1); and
wherein the genome of said lactic acid bacterium comprises genes encoding polypeptides having:
iii. lactose-specific phosphotransferase system (PTS) activity (EC 2.7.1.69)
iv. phospho-β-D-galactosidase activity (EC 3.2.1.85)
v. galactose-6-phosphate isomerase activity (EC 5.3.1.26),
vi. D-tagatose-6-phosphate kinase activity (EC 2.7.1.114), and
vii. tagatose 1,6-diphosphate aldolase activity (EC 4.1.2.40);
wherein the genome of said lactic acid bacterium is deleted for genes or lacks functional genes or genes encoding polypeptides having an enzymatic activity of:
viii. lactate dehydrogenase (E.C 1.1.1.27 or E.C. 1.1.1.28)
ix. phosphotransacetylase (E.C. 2.3.1.8) and
x. bifunctional alcohol dehydrogenase (E.C. 1.1.1.1 and EC 1.2.1.10).

2. A method for ethanol production according to claim 1, wherein the amino nitrogen source is acid hydrolysed corn steep liquor (CSLH) wherein the concentration of at least one free amino acid, selected from the group consisting on glutamine, histidine, methionine, leucine, isoleucine, and valine is at least 1.5 fold greater than the concentration of the corresponding amino acid in the original corn steep liquor from which the CSLH was derived.

3. A method for ethanol production according to claim 1 or 2, wherein the lactose content of the medium at step a) is from 20 g to 200 g lactose/L.

4. A method for ethanol production according to claim 2 or 3, wherein the CSLH w/v solids content of the medium at step a) is from 2% to 20%.

5. A method for ethanol production according to any one of claims 2 to 4, wherein the content of free histidine provided by the CSLH in the aqueous culture medium is at least 0.8 mM.

6. A method for ethanol production according to any one of claims 2 to 5, wherein the aqueous culture medium further comprises yeast extract.

7. A method for ethanol production according to any one of claims 1 to 6, wherein the culture is fed-batch; and wherein the culture in step b) is fed with at least lactose.

8. A method for ethanol production according to any one of claims 2 to 7, wherein the aqueous culture medium consists of the components: the residual whey permeate; the CSLH; water and optionally supplemented with yeast extract and/or an aqueous solution of lactose.

9. A method for ethanol production according to any one of claims 1 to 8, wherein the lactic acid bacteria belongs to a genus selected from the group consisting of Lactococcus, Lactobacillus, Pediococcus, Leuconostoc, Streptococcus, Oenococcus, and Bacillus.

10. A method for ethanol production according to any one of claims 1 to 9, wherein:
a. the polypeptide having pyruvate decarboxylase (PDC) activity (EC 4.1.1.1) has at least 80% sequence identity to an amino acid sequence of SEQ ID NO: 80; and
b. the polypeptide having alcohol dehydrogenase B activity (EC 1.1.1.1) has at least 80% sequence identity to an amino acid sequence of SEQ ID NO: 82.

11. A method for ethanol production according to any one of claims 1 to 10, wherein:
a. the amino acid sequence of the polypeptide having lactate dehydrogenase activity has at least 80% sequence identity to an amino acid sequence selected from among SEQ ID NO: 2, 4, 6, 8, 10, 12, 14, 16, 18, 20, 22, 24, 26, 28, 30, 32, 34 and 36;
b. the amino acid sequence of the polypeptide having phosphotransacetylase activity has at least 80% sequence identity to an amino acid sequence selected from among SEQ ID NO: 42, 44, 46, 48, 50, 52, 54, 56, 58 and 60;
c. the amino acid sequence of the polypeptide having bifunctional alcohol dehydrogenase activity has at least 80% sequence identity to an amino acid sequence selected from among SEQ ID NO: 62, 64, 66, 68, 70, 72, 74, 76 and 78.

12. A method for ethanol production according to any one of claims 1 to 11, wherein:
a. the lactose-specific phosphotransferase system (PTS) activity (EC 2.7.1.69) is provided by a first and a second polypeptide, wherein the amino acid sequence of the first polypeptide has at least 80% sequence identity to an amino acid sequence of SEQ ID NO: 84, and the amino acid sequence of the second polypeptide has at least 80% sequence identity to an amino acid sequence of SEQ ID NO: 86;
b. the amino acid sequence of the polypeptide having phospho-β-D-galactosidase activity (EC 3.2.1.85) has at least 80% sequence identity to an amino acid sequence of SEQ ID NO: 88;
c. the amino acid sequence of the polypeptide having galactose-6-phosphate isomerase activity (EC 5.3.1.26) is provided by a first and a second polypeptide, wherein the amino acid sequence of the first polypeptide has at least 80% sequence identity to an amino acid sequence of SEQ ID NO: 90 and the amino acid sequence of the second polypeptide has at least 80% sequence identity to an amino acid sequence of SEQ ID NO: 92;
d. the amino acid sequence of the polypeptide having D-tagatose-6-phosphate kinase activity (EC 2.7.1.114) has at least 80% sequence identity to an amino acid sequence of SEQ ID NO: 94; and
e. the amino acid sequence of the polypeptide having tagatose 1,6-diphosphate aldolase activity (EC 4.1.2.40) has at least 80% sequence identity to an amino acid sequence of SEQ ID NO: 96.

13. Use of a genetically engineered lactic acid bacterium for the production of ethanol from an aqueous culture medium comprising
I. whey premeate or residual whey permeate, and
II. an amino nitrogen source;
wherein the genetically engineered lactic acid bacterium comprises transgenes encoding:
i. a polypeptide having pyruvate decarboxylase (PDC) activity (EC 4.1.1.1); and
ii. a polypeptide having alcohol dehydrogenase B activity (EC 1.1.1.1); and
wherein the genome of said lactic acid bacterium comprises genes encoding polypeptides having:
iii. lactose-specific phosphotransferase system (PTS) activity (EC 2.7.1.69)
iv. phospho-β-D-galactosidase activity (EC 3.2.1.85)
v. galactose-6-phosphate isomerase activity (EC 5.3.1.26),
vi. D-tagatose-6-phosphate kinase activity (EC 2.7.1.114), and
vii. tagatose 1,6-diphosphate aldolase activity (EC 4.1.2.40); and
wherein the genome of said lactic acid bacterium is deleted for genes or lacks functional genes encoding polypeptides having an enzymatic activity of:
viii. lactate dehydrogenase (E.C 1.1.1.27 or E.C. 1.1.1.28)
ix. phosphotransacetylase (E.C. 2.3.1.8) and
x. bifunctional alcohol dehydrogenase (E.C. 1.1.1.1 and EC 1.2.1.10).

14. The use of a genetically engineered lactic acid bacterium for the production of ethanol according to claim 13, wherein the amino nitrogen source is acid hydrolysed corn steep liquor (CSLH), wherein the concentration of at least one free amino acid, selected from the group consisting on glutamine, histidine, methionine, leucine, isoleucine, and valine is at least 1.5 fold greater than the concentration of the corresponding amino acid in the original corn steep liquor from which the CSLH was derived.

15. A genetically engineered lactic acid bacterium for the production of ethanol from an aqueous culture medium comprising whey permeate or residual whey permeate, and an amino nitrogen source; wherein the genetically engineered lactic acid bacterium comprises transgenes encoding:
i. a polypeptide having pyruvate decarboxylase (PDC) activity (EC 4.1.1.1); and
ii. a polypeptide having alcohol dehydrogenase B activity (EC 1.1.1.1); and
wherein the genome of said lactic acid bacterium comprises genes encoding polypeptides having:
iii. lactose-specific phosphotransferase system (PTS) activity (EC 2.7.1.69)
iv. phospho-β-D-galactosidase activity (EC 3.2.1.85)
v. galactose-6-phosphate isomerase activity (EC 5.3.1.26),
vi. D-tagatose-6-phosphate kinase activity (EC 2.7.1.114), and
vii. tagatose 1,6-diphosphate aldolase activity (EC 4.1.2.40); and
wherein the genome of said lactic acid bacterium is deleted for genes or lacks functional genes or genes encoding polypeptides having an enzymatic activity of:
viii. lactate dehydrogenase (E.C 1.1.1.27 or E.C. 1.1.1.28)
ix. phosphotransacetylase (E.C. 2.3.1.8) and
x. bifunctional alcohol dehydrogenase (E.C. 1.1.1.1 and EC 1.2.1.10).
